(19) United States
(12) Patent Application Publication
(10) Pub. No.: US 2014/0142170 A1
(43) Pub. Date: May 22, 2014

(54) COMPOSITION AND METHOD FOR INFLUENCING ENERGY METABOLISM AND TREATING METABOLIC AND OTHER DISORDERS

(71) Applicants: Markku Laakso, Kuopio (FI); Nagendra Yaluri, Kuopio (FI)

(72) Inventors: Markku Laakso, Kuopio (FI); Nagendra Yaluri, Kuopio (FI)

(73) Assignee: SIRTUIN VALLEY OY, Helsinki (FI)

(21) Appl. No.: 14/004,178

(22) PCT Filed: Oct. 27, 2011
(86) PCT No.: PCT/FI2011/050945

§ 371 Date: Jan. 27, 2014

Related U.S. Application Data
(60) Provisional application No. 61/407,174, filed on Oct. 27, 2010.

(30) Foreign Application Priority Data
Oct. 27, 2010 (FI) 20106119

(51) Int. Cl. A61K 31/366; A61K 31/047; A61K 31/05; C07D 407/12; A61K 31/365

(52) U.S. Cl. CPC A61K 31/366; C07D 407/12; A61K 31/365; A61K 31/05; A61K 31/047
USPC 514/456; 549/299; 514/468; 514/470; 435/375; 549/305

(57) ABSTRACT

A composition and method for influencing energy metabolism and treating metabolic and other disorders is provided. A terpenoid lactone that is a selective activator of SIRT1 is generally in the form of a terpenoid dilactone having a 5-alkeny-loxy-furan-2- one group, such as strigolactone, GR 24, or another strigolactone analog, and is used as a therapeutic agent in a method for influencing energy metabolism and treating metabolic and other disorders. The terpenoid lactone may be administered as an individual agent or combined with a second compound such as a flavonoid, chalconoid, tannin, or nicotinamide inhibition antagonist.

### CROSS-REFERENCE TO RELATED APPLICATION

This application claims priority to Provisional U.S. Patent Application No. 61/407,174, filed Oct. 27, 2010, the disclosure of which is incorporated by reference herein.

### TECHNICAL FIELD

The present invention relates generally to compositions and methods for influencing energy metabolism. The invention additionally relates to compositions and methods for the treatment of metabolic and other disorders in a subject. The invention has utility in the fields of medicine and pharmacotherapy.

### BACKGROUND

Metabolic disorders, which are medical conditions characterized by problems with an organism's metabolism, are major health problems among humans. Since a healthy, functioning metabolism is crucial for life, metabolic disorders are treated very seriously.

The term “energy metabolism” refers to the energy changes that accompany biochemical reactions, particularly the reactions involved in the oxidation of metabolic fuels to provide energy linked to the formation of ATP (adenosine triphosphate) from ADP (adenosine diphosphate) and phosphate ions. The main sources of chemical energy for most organisms are carbohydrates, fats, and proteins; the energy that results from the oxidation of these nutrients sustains the biochemical reactions necessary for life. That is, the energy generated sustains the biosynthesis of cellular and extracellular components, the transport of ions and organic chemicals against concentration gradients, the conduction of electrical impulses in the nervous system, and the movement of cells as well as movement of the whole organism.

A significant aspect of a healthy metabolism is the generation of enzymes that break food down into energy and handle the transport of that energy. Most metabolic disorders are related to various types of enzyme malfunctions and can result in serious consequences. A metabolic disorder can cause a wide range of symptoms, including muscle weakness, neurological problems, intestinal irregularities, and cardiovascular problems, among many others. A metabolic disorder develops when some organs, such as the liver or pancreas, become diseased or do not function normally.

The treatments for metabolic disorders vary, depending on the nature of the specific disorder as well as the severity of the symptoms. Once the disorder has been identified, a doctor may prescribe drugs or therapy to help the body regulate itself. The patient may also be asked to participate in self-care through lifestyle changes such as an alteration in diet. Ideally, any treatment prescribes will cure or at least stabilize the metabolic disorder, allowing the patient to live a healthy, functional life.

The Silent Information Regulator (SIR) family of genes represents a highly conserved group of genes present in the genomes of organisms ranging from archaebacteria to eukaryotes, and have been found to be closely linked to many biological processes in the body that directly or indirectly relate to energy metabolism. The proteins encoded by members of the SIR gene family show high sequence conservation in a 250 amino acid core domain. A well-characterized gene in this family is S. cerevisiae Sir2. The Sir2 protein is an enzyme with histone deacetylase activity that requires NAD (nicotinamide adenine dinucleotide) as a co-substrate. The deacetylation of acetyl-lysine by Sir2 is coupled with NAD hydrolysis, producing nicotinamide and an acetyl-ADP ribose compound. Mammalian Sir2 homologs also exhibit NAD-dependent histone deacetylase activity.

In humans, there are seven Sir2-like genes, SIRT1 through SIRT7, that share the conserved catalytic domain of Sir2. SIRT1 is a nuclear protein with the highest degree of sequence similarity to Sir2. SIR1 regulates multiple cellular targets by deacetylation including the tumor suppressor p53, the cellular signaling factor NF-κB, and the FOXO transcription factors. SIRT3 is a homolog of SIRT1 that is conserved in prokaryotes and eukaryotes. The SIRT3 protein is targeted to the mitochondrial cristae by a unique domain located at the N-terminus. Like SIRT1, SIRT3 has NAD(+)-dependent protein deacetylase activity and is ubiquitously expressed, particularly in metabolically active tissues. Upon transfer to the mitochondria, SIRT3 is believed to be cleaved into a smaller, active form by a mitochondrial matrix processing peptidase (MPP).

Caloric restriction has been known for over 70 years to improve the health and extend the lifespan of mammals. Activation of the gene that encodes for human SIRT1 has been identified as the mechanism by which calorie restriction diets promote longevity. Certain compounds have also been identified as sirtuin activators, which increase the activity of sirtuins in the body. These compounds include, without limitation, resveratrol and other hydroxylated stilbenes such as pinosylvin. See, e.g., Howitz et al. (2003) Nature 425:191-196.

Resveratrol is a naturally occurring polyphonic phytoalexin that is mainly found in the skin of red grapes. It is known for its phytoestrogenic and antioxidant properties. Resveratrol has also been produced by chemical synthesis. Resveratrol increases SIRT1 activity and stimulates genes responsible for mitochondrial biogenesis in mice (Lagouge et al. (2006) Cell 127:1109-22). In humans, high SIRT1 mRNA expression has been found to be associated with high insulin sensitivity, as had been established previously with resveratrol-induced over-activation of SIRT1 in mice (Rutanen et al. (2010) Diabetes 59:829-35)).

Pinosylvin is a pre-infectious stilbenoid toxin, i.e., it is synthesized prior to infection, in contrast to a phytoalexin, which is synthesized during infection. It is present in the heartwood of Pinaceae, and serves as a fungitoxin protecting the wood from fungal infection.

International patent application WO 2009/090180 describes consumable products, which are produced by fermentation and contain pinosylvin and resveratrol.

US Patent Publication No. 2004/0259815 A1 describes compositions that can be given as dietary supplements and can contain hydroxylated stilbenes such as resveratrol, pinosylvin, and other inhibitors of different phases of the cell cycle.

US Patent Publication No. 2006/0276416 A1 relates to methods for treating or preventing drug-induced weight gain by administering to a subject a sirtuin-activating compound, which can be resveratrol or pinosylvin.

To date, resveratrol has been found to be the most potent naturally occurring compound capable of activating SIRT1. Nevertheless, the low bioavailability of resveratrol limits its utility as an orally or otherwise administered therapeutic agent in the context of monotherapy. There is a continued need for compounds and compositions that are effective in the treatment of many medical disorders, particularly metabolic disorders.

### SUMMARY OF THE INVENTION

The invention is addressed to the aforementioned need in the art and provides compositions and methods for influencing energy metabolism and treating metabolic and other disorders.

In one embodiment, a composition is provided that is useful in influencing energy metabolism and treating metabolic disorders. The composition comprises a unit dosage form containing a therapeutically effective unit dosage of a terpenoid lactone that is a selective activator of SIRT1. An “activator of SIRT1” as used herein refers to a compound that activates the SIRT1 enzyme in the body, i.e., increases the activity of the enzyme as will be explained in detail infra. By “selective” in this context is meant that the terpenoid lactone upregulates the SIRT1 energy metabolism pathway but does not activate to any significant degree the energy metabolism pathway regulated by 5′ AMP-activated protein kinase, or “AMPK.” More specifically, using the assay described in Example 8, involving treatment of eukaryotic cells with a predetermined quantity of a terpenoid lactone as “test” compound, a compound is determined to be a selective activator of SIRT1 if there is a statistically significant increase in the expression of SIRT1 protein but no statistically significant increase in the expression of phosphorylated AMPK, or “pAMPK.” Nonselective SIRT1 activators include, by way of example, stilbenoids such as resveratrol, pinosylvin, and the like, insofar as these compounds activate both SIRT1 and AMPK. The terpenoid lactone that serves as the selective activator of SIRT1 in this embodiment is generally a dilactone having a substituted or unsubstituted 5-alkenyloxy-furan-2-one segment in its molecular structure, such as is present in strigolactone and strigolactone analogs.

Strigolactones are newly identified plant hormones, which participate in the regulation of lateral shoot branching and root development in plants. It has been shown that a strigolactone analog (GR 24) causes a rapid increase in NADH concentration, NADH dehydrogenase activity, and the ATP content of the fungal cell. The core molecular structure of strigolactone and its naturally occurring analogs is as follows:

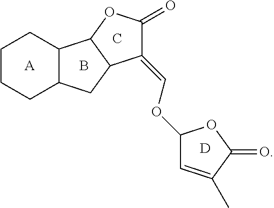

The invention also provides a method for influencing energy metabolism in a eukaryotic cell, wherein the method comprises contacting the cell with a terpenoid lactone that is a selective activator of SIRT1 in an amount effective to influence energy metabolism. In a related embodiment, a method for influencing energy metabolism is provided that involves contacting the cell with the aforementioned terpenoid lactone in combination with an additional SIRT1 activator, e.g., a nonselective SIRT1 activator such as resveratrol, pinosylvin, or the like, each in amount effective to influence energy metabolism in a eukaryotic cell.

The invention additionally provides a method for treating a metabolic disorder in a subject, by administering to a subject afflicted with or prone to the disorder a therapeutically effective amount of a terpenoid lactone that is a selective activator of SIRT1. In a related embodiment, the method for treating a metabolic disorder involves administering the aforementioned terpenoid lactone in combination with an additional SIRT1 activator, which may be a nonselective SIR1 activator such as resveratrol, pinosylvin, or the like. The metabolic disorder treated may be Type 2 diabetes or obesity. The metabolic disorder may also be “Metabolic Syndrome,” also referred to as “Syndrome X” and “Metabolic Syndrome X,” or it may be any one or more of the conditions associated with Metabolic Syndrome, including, without limitation, hypertension, insulin resistance, and dyslipidemia. The metabolic disorder may also involve various aspects of the aging process as well as adverse skin conditions, particularly those adverse skin conditions associated with aging.

Furthermore, the present invention provides a pharmaceutical substance or dietary supplement or nutritive substance in the form of a packaged pharmaceutical preparation, where the packaged preparation includes, in one embodiment, at least one dosage form containing a terpenoid lactone that is a selective activator of SIRT1. In a related embodiment, the packaged preparation includes at least one dosage form containing the aforementioned terpenoid lactone and at least one additional dosage form containing another activator of SIRT1, which may be a nonselective activator such as resveratrol, pinosylvin, or the like. In a further related embodiment, the packaged pharmaceutical preparation contains at least one dosage form containing a combination of the terpenoid lactone and, the additional SIRT1 activator. In still a further related embodiment, the packaged pharmaceutical preparation contains a plurality of unit dosage forms each containing a therapeutically effective unit dosage of the terpenoid lactone and a therapeutically effective unit dosage of another SIRT1 activator. The packaged pharmaceutical preparation also includes instructions to patients for self-administration of the dosage forms as dietary supplements.

The use of a terpenoid lactone that is a selective activator of SIRT1, such as GR 24, as well as the use of such a terpenoid lactone with an additional SIRT1 activator that may or may not be a selective SIRT1 activator, has been found to be unexpectedly effective as a composition for influencing energy metabolism, as there is no suggestion in the art that a terpenoid lactone that is a selective activator of SIRT1, alone or in combination with an additional SIRT1 activator, would be effective in treating disorders such as those associated with energy metabolism, energy expenditure, mitochondrial biogenesis, or insulin sensitivity.

The invention also provides certain terpenoid lactones as novel compounds having unique molecular structures and useful, inter alia, as SIRT1 activators:

In one embodiment, novel terpenoid lactones are provided that have the structure of formula (I)

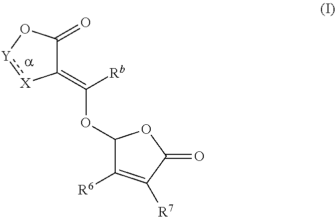

wherein:

α is an optionally present double bond;

when α is present, such that X and Y are linked through a double bond, X is CR¹ and Y is CR³;

when α is absent, such that X and Y are linked through a single bond, X is selected from CR¹R² and CR¹R²—CR⁸R⁹, and Y is CR³R⁴;

R¹, R², R³, R⁴, R⁸, and R⁹ are independently selected from hydrogen, halo, hydroxyl, sulfhydryl, C₁-C₂₄ alkoxy, C₂-C₂₄ alkenyloxy, C₂-C₂₄ alkynyloxy, C₅-C₂₄ aryloxy, C₂-C₂₄ alkylcarbonyl, C₆-C₂₄ arylcarbonyl, C₂-C₂₄ alkylcarbonyloxy, C₆-C₂₄ arylcarbonyloxy, halocarbonyl, C₂-C₂₄ alkylcarbonato, C₆-C₂₄ arylcarbonato, carboxy, carboxylato, carbamoyl, mono-(C₁-C₂₄ alkyl)-substituted carbamoyl, di-(C₁-C₂₄ alkyl)-substituted carbamoyl, mono-(C₆-C₂₄ aryl)-substituted carbamoyl, thiocarbamoyl, carbamido, cyano, isocyano, cyanato, isocyanato, isothiocyanato, azido, formyl, thioformyl, amino, mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido, C₆-C₂₄ arylamido, imino, alkylimino, arylimino, nitro, nitroso, sulfo, sulfonato, C₁-C₂₄ alkylthio, C₅-C₂₄ arylthio, C₁-C₂₄ alkylsulfinyl, C₅-C₂₄ arylsulfinyl, C₁-C₂₄ alkylsulfonyl, C₅-C₂₄ arylsulfonyl, phosphono, phosphonato, phosphinato, phosphono, phosphino, C₁-C₂₄ alkyl, C₂-C₂₄ alkenyl, C₂-C₂₄ alkynyl, C₅-C₂₄ aryl, C₆-C₂₄ alkaryl, and C₆-C₂₄ aralkyl, and further wherein R¹ and R³, and R¹ and R⁸ may be taken together to form a cyclic structure selected from a five-membered ring and a six-membered ring, optionally fused to an additional five-membered or six-membered ring, wherein the rings are aromatic, alicyclic, heteroaromatic, or heteroalicyclic, and have zero to 4 non-hydrogen substituents and zero to 3 heteroatoms;

R⁵ is selected from hydrogen, halo, C₁-C₆ alkyl, substituted C₁-C₆ alkyl, C₁-C₆ heteroalkyl, and substituted C₁-C₆ heteroalkyl; and

(a) R⁶ and R⁷ taken together form a C₅-C₁₄ cyclic group, optionally substituted and/or containing at least one heteroatom; or

(b) R⁶ is hydrogen and R⁷ is selected from halo, hydroxy, C₁-C₁₂ alkoxy, C₂-C₁₂ hydrocarbyl, substituted C₂-C₁₂ hydrocarbyl, heteroatom-containing C₂-C₁₂ hydrocarbyl, and substituted heteroatom-containing C₂-C₁₂ hydrocarbyl; or

(c) R⁶ is selected from halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, and R⁷ is selected from hydrogen, halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₇ hydrocarbyl, wherein R⁶ and R⁷ may be the same or different.

In another embodiment, novel terpenoid lactones are provided that have the structure of formula (VIII)

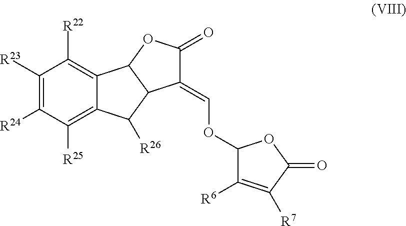

wherein:

R⁶ and R⁷ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, hydrocarbyl, substituted C₁-C₁₂ hydrocarbyl, heteroatom-containing C₁-C₁₂ hydrocarbyl, and substituted heteroatom-containing C₁-C₁₂ hydrocarbyl, or R⁶ and R⁷ may be taken together to form a C₅-C₁₄ cyclic group, optionally substituted and/or containing at least one heteroatom;

R²¹ is selected from hydrogen, hydroxy, C₁-C₃ alkoxy, and C₂-C₄ acyloxy; and either

(a) one of R²², R²³, R²⁴, and R²⁵ is C₁-C₁₂ hydrocarbyl, optionally substituted and optionally heteroatom-containing, and the others are hydrogen; or

(b) R²², R²³, R²⁴, and R²⁵ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, substituted C₁-C₁₂ hydrocarbyl, heteroatom-containing C₁-C₁₂ hydrocarbyl, and substituted heteroatom-containing C₁-C₁₂ hydrocarbyl, with the proviso that at least one R²², R²³, R²⁴, and R²⁵ is optionally substituted, optionally heteroatom-containing C₁-C₁₂ hydrocarbyloxy.

### BRIEF DESCRIPTION OF THE DRAWINGS

FIG. 1 depicts immunoblots and densitometry results of immunoblots from cellular lysates of 3T3L1 preadipocytes treated with 100 μM GR 24 for 24 hours, as described in Example 8. FIG. 1A shows the increase in SIRT1 protein expression after treatment with GR 24. FIG. 1B depicts the activation of PGC1, a master regulator of mitochondrial biogenesis. FIG. 1C shows the down-regulation of phospho-AMPK when compared to control. FIG. 1D represents the AMPK protein levels. FIG. 1E depicts the decrease in phospho-ACC protein expression. FIG. 1F shows the protein expression of ACC, a downstream target of AMPK. FIG. 1G shows the immunoblot of α-tubulin.

FIG. 2 depicts SIRT1 immunoblot and densitometry results from 3T3 L1 preadipocytes treated with 60 μM resveratrol and 60 μM GR 24 for 24 hours, as described in Example 9. FIG. 2A depicts the significant increase of SIRT1 protein expression treated with GR 24 compared to control. FIG. 2B shows the immunoblots of SIRT1. FIG. 2C shows the immunoblot of α-tubulin.

FIG. 3 depicts immunoblots and densitometry from 3T3 L1 preadipocytes treated with 60 μM resveratrol and 60 μM GR 24 for 24 hours, as described in Example 10. FIG. 3A shows densitometry of phospho-AMPK, which shows a significant increase in expression with resveratrol but not with GR 24. FIG. 3B shows AMPK expression in the same blot obtained after stripping and reprobing. FIG. 3C represents the western blot image of phospho-AMPK. FIG. 3D shows the western blot image of AMPK and FIG. 3E shows the western blot image of α-tubulin.

FIG. 4 depicts immunoblots and densitometry from 3T3 L1 preadipocytes treated with 60 μM resveratrol and 60 μM GR 24 for 24 hours, as described in Example 11. FIG. 4A indicates that there is no change in phospho-ACC expression compared to control. FIG. 4B shows the expression level of ACC. FIG. 4C represents the immunoblot of phospho-ACC. FIG. 4D shows the immunoblot of ACC and FIG. 4E shows the immunoblot of α-tubulin.

FIG. 5 depicts mitochondrial staining in 3T3L1 preadipocytes treated with 60 μM resveratrol (FIG. 5B) and GR 24 (Strigolactone) (FIG. 5C) compared to Control (FIG. 5A), as described in Example 12.

FIG. 6 depicts SIRT1 expression in 3T3 L1 cells treated with 60 μM GR 24 alone or in combination with GR 24 and resveratrol, GR 24 and pinosylvin, or GR 24 and resveratrol and pinosylvin, as described in Example 13. FIG. 6A shows that SIRT1 protein expression was significantly (*P<0.05) increased with all the treatments compared to control. A significant increase in SIRT1 (*P<0.05) was also observed when GR 24 treated cells were compared with GR 24 and resveratrol treatment. FIG. 6B depicts corresponding Western blotting results of SIRT1 and tubulin (used as loading control).

FIG. 7 depicts Western blots and densitometry from 3T3 L1 preadipocytes treated with 60 μM GR 24 alone or in combination with GR 24 and resveratrol, GR 24 and pinosylvin, or GR 24 and resveratrol and pinosylvin, as described in Example 14. AMPK-activation expression levels are presented. FIG. 7A depicts AMPK activation (pAMPK/AMPK/α-tubulin ratio) in cultured 3T3 L1 cells treated with 60 μM GR 24 alone or the foregoing combinations. FIG. 7B depicts corresponding Western blotting results of pAMPK, AMPK and α-tubulin (used as loading control).

FIG. 8 illustrates the mechanism involved in the activation of SIRT1 and mitochondrial biogenesis by GR 24.

FIG. 9 provides SIRT1 densitometry and immunoblot results from 3T3 L1 preadipocytes treated with 60 μM GR24 or 60 μM (1A) or 60 μM (1B) for 24 hours, as described in Example 15. FIG. 9A is a graph showing the mean SIRT1 protein expression from one experiment with two replicates. FIG. 9B shows the immunoblots of SIRT1 and α-tubulin.

FIG. 10 provides SIRT1 densitometry and immunoblot results from 3T3 L1 preadipocytes treated with 60 μM GR24 or 60 μM (3A) or 60 μM (3B) for 24 hours, as described in Example 15. FIG. 10A is a graph showing the mean SIRT1 protein expression from one experiment with two replicates. FIG. 10B shows the immunoblots of SIRT1 and α-tubulin.

FIG. 11 provides SIRT1 densitometry and immunoblot results from 3T3 L1 preadipocytes treated with 60 μM GR24 or 60 μM (4A) or 60 μM (4B) for 24 hours, as described in Example 15. FIG. 11A is a graph showing the mean SIRT1 protein expression from one experiment with two replicates. FIG. 11B shows the immunoblots of SIRT1 and α-tubulin.

FIG. 12 provides SIRT1 densitometry and immunoblot results from 3T3 L1 preadipocytes treated with 60 μM GR24 or 60 μM (5A) or 60 μM (5B) for 24 hours, as described in Example 15. FIG. 12A is a graph showing the mean SIRT1 protein expression from one experiment with two replicates. FIG. 12B shows the immunoblots of SIRT1 and α-tubulin.

FIG. 13 provides SIRT1 densitometry and immunoblot results from 3T3 L1 preadipocytes treated with 60 μM GR24 or 60 μM (6A) or 60 μM (6B) for 24 hours, as described in Example 15. FIG. 13A is a graph showing the mean SIRT1 protein expression from one experiment with two replicates. FIG. 13B shows the immunoblots of SIRT1 and α-tubulin.

FIG. 14 provides SIRT1 densitometry and immunoblot results from 3T3 L1 preadipocytes treated with 60 μM GR24 or 60 μM (7A) or 60 μM (7B) for 24 hours, as described in Example 15. FIG. 14A is a graph showing the mean SIRT1 protein expression from one experiment with two replicates. FIG. 14B shows the immunoblots of SIRT1 and α-tubulin.

FIG. 15 provides SIRT1 densitometry and immunoblot results from 3T3 L1 preadipocytes treated with 60 μM GR24 or 60 μM (8A) or 60 μM (8B) for 24 hours, as described in Example 15. FIG. 15A is a graph showing the mean SIRT1 protein expression from one experiment with two replicates. FIG. 15B shows the immunoblots of SIRT1 and α-tubulin.

FIG. 16 provides SIRT1 densitometry and immunoblot results from 3T3 L1 preadipocytes treated with 60 μM GR24 or 60 μM (1A) for 24 hours, as described in Example 15. FIG. 16A is a graph illustrating the mean±SEM of SIRT1 protein expression from three independent experiments with a total of eight replicates. FIG. 16B shows the immunoblots of SIRT1 and α-tubulin.

FIG. 17 provides SIRT1 densitometry and immunoblot results from 3T3 L1 preadipocytes treated with 60 μM GR24 or 60 μM (1B) for 24 hours, as described in Example 15. FIG. 17A is a graph illustrating the mean±SEM of SIRT1 protein expression from three independent experiments with a total of eight replicates. FIG. 17B shows the immunoblots of SIRT1 and α-tubulin.

FIG. 18 provides SIRT1 densitometry and immunoblot results from 3T3 L1 preadipocytes treated with 60 μM GR24 or 60 μM (5A) for 24 hours, as described in Example 15. FIG. 18A is a graph illustrating the mean±SEM of SIRT1 protein expression from three independent experiments with a total of eight replicates. FIG. 18B shows the immunoblots of SIRT1 and α-tubulin.

FIG. 19 provides SIRT1 densitometry and immunoblot results from 3T3 L1 preadipocytes treated with 60 μM GR24 or 60 μM (5B) for 24 hours, as described in Example 15. FIG. 19A is a graph illustrating the mean±SEM of SIRT1 protein expression from three independent experiments with a total of eight replicates. FIG. 19B shows the immunoblots of SIRT1 and α-tubulin.

FIG. 20 provides SIRT1 densitometry and immunoblot results from 3T3 L1 preadipocytes treated with 60 μM GR24 or 60 μM (6A) for 24 hours, as described in Example 15. FIG. 20A is a graph illustrating the mean±SEM of SIRT1 protein expression from three independent experiments with a total of eight replicates. FIG. 20B shows the immunoblots of SIRT1 and α-tubulin.

FIG. 21 provides SIRT1 densitometry and immunoblot results from 3T3 L1 preadipocytes treated with 60 μM GR24 or 60 μM (7A) for 24 hours, as described in Example 15. FIG. 21A is a graph illustrating the mean±SEM of SIRT1 protein expression from three independent experiments with a total of eight replicates. FIG. 21B shows the immunoblots of SIRT1 and α-tubulin.

FIG. 22 provides PGC-1α densitometry and immunoblot results from 3T3 L1 preadipocytes treated with 60 μM GR24 or 60 μM (1A) for 24 hours, as described in Example 15. FIG. 22A is a graph illustrating the mean±SEM of PGC-1α protein expression from two independent experiments with a total of six replicates. FIG. 22B shows the immunoblots of PGC-1α and α-tubulin.

FIG. 23 provides PGC-1α densitometry and immunoblot results from 3T3 L1 preadipocytes treated with 60 μM GR24 or 60 μM (1B) for 24 hours, as described in Example 15. FIG. 23A is a graph illustrating the mean±SEM of PGC-1α protein expression from two independent experiments with a total of six replicates. FIG. 23B shows the immunoblots of PGC-1α and α-tubulin.

FIG. 24 provides PGC-1α densitometry and immunoblot results from 3T3 L1 preadipocytes treated with 60 μM GR24 or 60 μM (5A) for 24 hours, as described in Example 15. FIG. 24A is a graph illustrating the mean±SEM of PGC-1α protein expression from two independent experiments with a total of six replicates. FIG. 24B shows the immunoblots of PGC-1α and α-tubulin.

FIG. 25 provides PGC-1α densitometry and immunoblot results from 3T3 L1 preadipocytes treated with 60 μM GR24 or 60 μM (5A) for 24 hours, as described in Example 15. FIG. 25A is a graph illustrating the mean±SEM of PGC-1α protein expression from two independent experiments with a total of six replicates. FIG. 25B shows the immunoblots of PGC-1α and α-tubulin.

FIG. 26 provides PGC-1α densitometry and immunoblot results from 3T3 L1 preadipocytes treated with 60 μM GR24 or 60 μM (6A) for 24 hours, as described in Example 15. FIG. 26A is a graph illustrating the mean±SEM of PGC-1α protein expression from two independent experiments with a total of six replicates. FIG. 26B shows the immunoblots of PGC-1α and α-tubulin.

FIG. 27 provides PGC-1α densitometry and immunoblot results from 3T3 L1 preadipocytes treated with 60 μM GR24 or 60 μM (7A) for 24 hours, as described in Example 15. FIG. 27A is a graph illustrating the mean±SEM of PGC-1α protein expression from two independent experiments with a total of six replicates. FIG. 27B shows the immunoblots of PGC-1α and α-tubulin.

FIG. 28 provides SIRT1 densitometry and immunoblot results from MIN6 cells treated with 60 μM GR24 for 24 hours at 5 mM glucose. FIG. 28A is a graph illustrating the mean±SEM of SIRT1 protein expression from two independent experiments, with a total of six replicates. FIG. 28B shows the immunoblots of SIRT1 and Actin.

FIG. 29 provides PGC-1α densitometry and immunoblot results from MIN6 cells treated with 60 μM GR24 for 24 hours at 5 mM glucose. FIG. 29A is a graph illustrating the mean±SEM of PGC-1α protein expression from two independent experiments, with a total of six replicates. FIG. 29B shows the immunoblots of PGC-1α and Actin.

FIG. 30 provides pAMPK densitometry and immunoblot results from MIN6 cells treated with 60 μM GR24 for 24 hours at 5 mM glucose. FIG. 30A is a graph illustrating the mean±SEM of pAMPK protein expression from two independent experiments, with a total of six replicates. FIG. 30B shows the immunoblots of pAMPK and Actin.

FIG. 31 provides AMPK densitometry and immunoblot results from MIN6 cells treated with 60 μM GR24 for 24 hours at 5 mM glucose. FIG. 31A is a graph illustrating the mean±SEM of AMPK protein expression from two independent experiments, with a total of six replicates. FIG. 30B shows the immunoblots of AMPK and Actin.

FIG. 32 provides SIRT1 densitometry and immunoblot results from MIN6 cells treated with 60 μM GR24 for 24 hours at 25 mM glucose. FIG. 32A is a graph illustrating the mean±SEM of SIRT1 protein expression from two independent experiments, with a total of six replicates. FIG. 32B shows the immunoblots of SIRT1 and Actin.

FIG. 33 provides PGC-1α densitometry and immunoblot results from MIN6 cells treated with 60 μM GR24 for 24 hours at 25 mM glucose. FIG. 33A is a graph illustrating the mean±SEM of PGC-1α protein expression from two independent experiments, with a total of six replicates. FIG. 33B shows the immunoblots of PGC-1a and Actin.

FIG. 34 provides pAMPK densitometry and immunoblot results from MIN6 cells treated with 60 μM GR24 for 24 hours at 25 mM glucose. FIG. 34A is a graph illustrating the mean±SEM of pAMPK protein expression from two independent experiments, with a total of six replicates. FIG. 34B shows the immunoblots of pAMPK and Actin.

FIG. 35 provides AMPK densitometry and immunoblot results from MIN6 cells treated with 60 μM GR24 for 24 hours at 25 mM glucose. FIG. 35A is a graph illustrating the mean±SEM of AMPK protein expression from two independent experiments, with a total of six replicates. FIG. 35B shows the immunoblots of AMPK and Actin.

FIG. 36 provides SIRT1 densitometry and immunoblot results from 3T3 L1 preadipocytes treated with 10 μM GR24 or 10 μM (5A) for 24 hours, as described in Example 15. FIG. 36A is a graph illustrating the mean±SEM of SIRT1 protein expression from three independent experiments with a total of eight replicates. FIG. 36B shows the immunoblots of SIRT1 and α-tubulin.

FIG. 37 provides SIRT1 densitometry and immunoblot results from 3T3 L1 preadipocytes treated with 20 μM GR24 or 20 μM (5A) for 24 hours, as described in Example 15. FIG. 37A is a graph illustrating the mean±SEM of SIRT1 protein expression from three independent experiments with a total of eight replicates. FIG. 37B shows the immunoblots of SIRT1 and α-tubulin.

### DETAILED DESCRIPTION OF THE INVENTION

### I. Definitions and Nomenclature:

Unless defined otherwise, all technical and scientific terms used herein have the meaning commonly understood by one of ordinary skill in the art to which the invention pertains. Specific terminology of particular importance to the description of the present invention is defined below.

In this specification and the appended claims, the singular forms “a,” “an” and “the” include plural referents unless the context clearly dictates otherwise. Thus, for example, “a terpenoid lactone” refers not only to a single terpenoid lactone but also to a combination of two or more different terpenoid lactones, “a SIRT1 activator” refers to a single SIRT1 activator or to a combination of SIRT1 activators, “a pharmaceutically acceptable carrier” refers to a combination of pharmaceutically acceptable carriers, as will usually be the case, as well as to a single pharmaceutically acceptable carrier.

When referring to an active agent, whether specified as a particular compound (e.g., demethylsorgolactone) or a compound class (e.g., a terpenoid lactone), the term used to refer to the agent is intended to encompass not only the specified molecular entity but also its pharmaceutically acceptable, pharmacologically active analogs and derivatives, including, but not limited to, salts, esters, amides, prodrugs, conjugates, active metabolites, hydrates, crystalline forms, enantiomers, stereoisomers, and other such derivatives, analogs, and related compounds.

The terms “treating” and “treatment” as used herein refer to reduction in severity and/or frequency of symptoms, elimination of symptoms and/or underlying cause, and improvement or remediation of damage. Unless otherwise indicated, the terms “treating” and “treatment” as used herein encompass prevention of symptoms or the occurrence of a metabolic disorder, such as in an individual who may be predisposed to such symptoms or disorders.

The terms “effective amount” and “therapeutically effective amount” of an agent, compound, composition or combination of the invention refer to an amount that is nontoxic and effective for producing some a therapeutic effect upon administration to a subject.

The term “dosage form” denotes any form of a pharmaceutical composition that contains an amount of active agent sufficient to achieve a therapeutic effect with a single administration. When the formulation is an orally administered tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release, modified release and delayed release formulations. “Controlled release” includes “sustained release” (synonymous with “extended release”), referring to a formulation that provides for gradual release of an active agent over an extended period of time, and that preferably, although not necessarily, results in substantially constant blood levels of an agent over an extended time period. “Controlled release” also includes “delayed release,” indicating a formulation that, following administration to a patient, provides for a measurable time delay before the active agent is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug administration. The term “pharmaceutically acceptable salts” include acid addition salts of basic agents which are formed with inorganic acids such as, for example, hydrochloric or phosphoric acids, or with organic acids such as acetic, oxalic, tartaric, mandelic acids, and the like. Pharmaceutically acceptable basic addition salts of acidic agents can be prepared with inorganic bases such as, for example, sodium, potassium, ammonium, calcium, or ferric hydroxides, or with organic bases such as isopropylamine, trimethylamine, histidine, procaine and the like.

“Pharmacologically active” (or simply “active”) as in a “pharmacologically active” analog, refers to a compound having the same type of pharmacological activity as the parent compound and approximately equivalent in degree.

As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject is normally human, but may also be a domestic livestock, laboratory subject or pet animal.

As used herein, the phrase “having the formula” or “having the structure” is not intended to be limiting and is used in the same way that the term “comprising” is commonly used.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred lower alkyl substituents contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred lower alkoxy substituents contain 1 to 3 carbon atoms, and particularly preferred such substituents contain 1 or 2 carbon atoms (i.e., methoxy and ethoxy). The terms “alkenyloxy” and “alkynyloxy” are defined in an analogous manner.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctyinaphthyl, 3-ethyl-cyclopenta-1,4-diene, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The term “cyclic” refers to alicyclic or aromatic substituents that may or may not be substituted and/or heteroatom containing, and that may be monocyclic, bicyclic, or polycyclic.

The term “alicyclic” is used in the conventional sense to refer to an aliphatic cyclic moiety, as opposed to an aromatic cyclic moiety, and may be monocyclic, bicyclic, or polycyclic.

The terms “halo” and “halogen” are used in the conventional sense to refer to a chloro, bromo, fluoro, or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur, preferably nitrogen or oxygen. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

When a functional group is termed “protected,” this means that the group is in modified form to preclude undesired side reactions at the protected site. Suitable protecting groups for the compounds of the present invention will be recognized from the present application taking into account the level of skill in the art, and with reference to standard textbooks, such as Greene et al., Protective Groups in Organic Synthesis (New York: Wiley, 1991).

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N—(C₁-C₂₄ alkyl), N—(C₆-C₂₄ aryl)-substituted carbamoyl, thiocarbamoyl (—(CS)—NH₂), carbamido (—NH—(CO)—NH₂), cyano(—C≡N), isocyano (—N⁺≡C⁻—), cyanato (—O—C≡N), isocyanato (—O—N⁺≡C⁻—), isothiocyanato (—S—C≡N), azido (—N═N⁺≡N⁻), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₄ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present. Similarly, the phrase an “optionally present” bond as indicated by a dotted line - - - in the chemical formulae herein means that a bond may or may not be present.

### II. Compounds and Compositions:

In a first aspect of the invention, a pharmaceutical composition is provided as a unit dosage form containing a therapeutically effective amount of a terpenoid lactone that is a selective activator of SIRT1. A “unit dosage form” as used herein refers to a discrete dosage form that contains a single dose of the therapeutic agent, as that term is conventionally used in the fields of pharmaceutical preparation and drug delivery. The selective SIRT1 activator is a terpenoid lactone that measurably increases the activity of SIRT1 in a cell, particularly a eukaryotic cell, and/or in the body.

More specifically, a “SIRT1 activator” as that term is used herein refers to a compound or composition that increases the level of the SIRT1 protein and/or increases at least one activity of SIRT1 by at least about 10%, 25%, 50%, or more. Examples of SIRT1 activity in this context include, without limitation, deacetylating histones, increasing genomic stability, and silencing transcription. “Selective” SIRT1 activators are compounds that activate SIRT1 “selectively” relative to activation of AMPK, as explained earlier herein.

The terpenoid lactone is generally a dilactone that contains a 5-alkenyloxy-furan-2-one group, i.e., a molecular segment having the structure

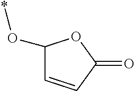 [SMILES: *OC1C=CC(=O)O1]

where the “*” represents the point of attachment to the remainder of the molecule, and where the unsubstituted carbon atoms in the segment shown can be substituted with one or more non-hydrogen substituents. For example, a terpenoid lactone useful in the compositions and methods of the invention may have the structure of formula (I)

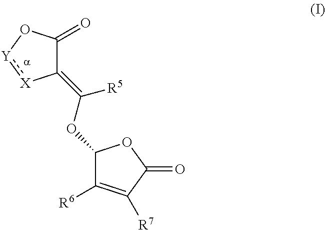

wherein:

α is an optionally present double bond;

when α is present, such that X and Y are linked through a double bond, X is CR¹ and Y is CR³;

when α is absent, such that X and Y are linked through a single bond, X is selected from CR¹R² and CR¹R²—CR⁸R⁹, and Y is CR³R⁴;

R¹, R², R³, R⁴, R⁸, and R⁹ are independently selected from hydrogen, halo, hydroxyl, sulfhydryl, C₁-C₂₄ alkoxy, C₂-C₂₄ alkenyloxy, C₂-C₂₄ alkynyloxy, C₅-C₂₄ aryloxy, C₂-C₂₄ alkylcarbonyl, C₆-C₂₄ arylcarbonyl, C₂-C₂₄ alkylcarbonyloxy, C₆-C₂₄ arylcarbonyloxy, halocarbonyl, C₂-C₂₄ alkylcarbonato, C₆-C₂₄ arylcarbonato, carboxy, carboxylato, carbamoyl, mono-(C₁-C₂₄ alkyl)-substituted carbamoyl, di-(C₁-C₂₄ alkyl)-substituted carbamoyl, mono-(C₆-C₂₄ aryl)-substituted carbamoyl, thiocarbamoyl, carbamido, cyano, isocyano, cyanato, isocyanato, isothiocyanato, azido, formyl, thioformyl, amino, mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido, C₆-C₂₄ arylamido, imino, alkylimino, arylimino, nitro, nitroso, sulfo, sulfonato, C₁-C₂₄ alkylthio, C₅-C₂₄ arylthio, C₁-C₂₄ alkylsulfinyl, C₅-C₂₄ arylsulfinyl, C₁-C₂₄ alkylsulfonyl, C₅-C₂₄ arylsulfonyl, phosphono, phosphonato, phosphinato, phosphono, phosphino, C₁-C₂₄ alkyl, C₂-C₂₄ alkenyl, C₂-C₂₄ alkynyl, C₅-C₂₄ aryl, C₆-C₂₄ alkaryl, and C₆-C₂₄ aralkyl, and further wherein R¹ and R³, and R¹ and R⁸ may be taken together to form a cyclic structure selected from a five-membered ring and a six-membered ring, optionally fused to an additional five-membered or six-membered ring, wherein the rings are aromatic, alicyclic, heteroaromatic, or heteroalicyclic, and have zero to 4 non-hydrogen substituents and zero to 3 heteroatoms;

R⁵ is selected from hydrogen, halo, C₁-C₆ alkyl, substituted C₁-C₆ alkyl, C₁-C₆ heteroalkyl, and substituted C₁-C₆ heteroalkyl; and

R⁶ and R⁷ are independently selected from hydrogen, halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, or R⁶ and R⁷ may be taken together to form a C₅-C₁₄ cyclic group, optionally substituted and/or containing at least one heteroatom.

Accordingly, in those structures wherein α is present as a double bond linking X and Y, wherein X is CR¹ and Y is CR³, it will be appreciated that such compounds may be represented by the structure of formula (II)

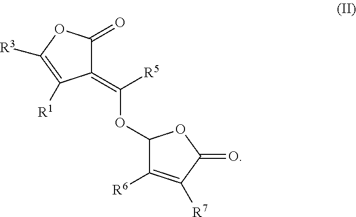

In those structures wherein α is not present, such that a single bond links X and Y, such that X is CR¹R² or CR¹R²—CR⁸R⁹, and Y is CR³R⁴, such compounds having the structures of formula (III) or formula (IV), respectively

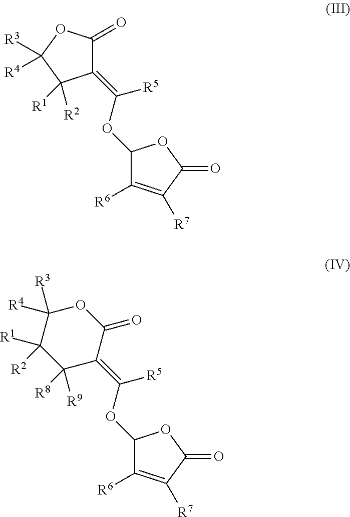

In certain embodiments, the terpenoid lactones have the structure of formula (III) wherein R¹ and R³ are linked together to form an additional five-membered or six-membered ring optionally fused to an additional five-membered or six-membered ring, which is in turn optionally fused to another five-membered or six-membered ring, wherein the rings are aromatic, partially aromatic, alicyclic, heteroaromatic, or heteroalicyclic, and have zero to 4 non-hydrogen substituents and zero to 3 heteroatoms. These compounds are illustrated by the structures of formula (V) and (VI)

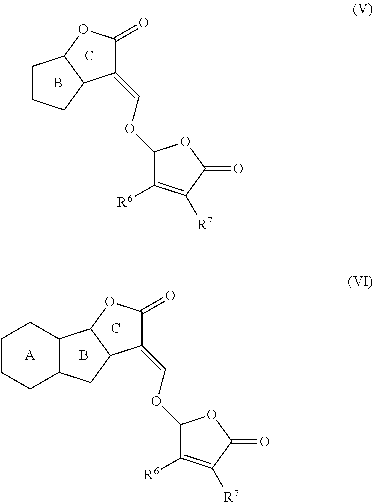

with regard to the chemical structure of natural strigolactones, and wherein rings A, B, and C may contain unsaturated bonds and substituents as indicated above. In preferred such compounds, R², R⁴, and R⁵ are hydrogen, and R⁶ and R⁷ are independently selected from hydrogen, C₁-C₆ alkoxy, and C₁-C₆ alkyl. In particularly preferred such compounds, one of R⁶ and R⁷ is hydrogen and the other is C₁-C₃ alkyl, e.g., methyl.

It will be appreciated that all of the terpenoid lactones described may be in the form of a single stereoisorner, i.e., be “stereoisornerically pure,” or contained in a mixture of two or more stereoisomers, e.g., two diastereomers, two enantiomers, or, more typically herein, a mixture of two diastereomers and two enantiomers. That is, the terpenoid lactones have an asymmetric carbon atom bound to the ether oxygen atom between the two lactone rings, meaning that the compound may be in the form of either of two enantiomers, or may be a racemic mixture thereof. In addition, the two additional stereogenic centers linking rings “B” and “C” give rise to two diastereomeric forms at that location. Compound (V), for instance, can have any of the following configurations (V-C1), (V-C2), (V-C3), and (V-C4)

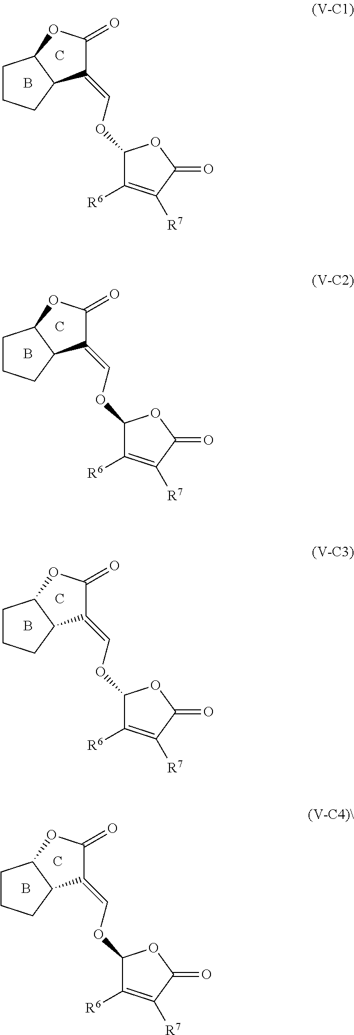

while compound (VI), as another example, can have any of the following configurations (VI-C1), (VI-C2), (VI-C3), and (VI-C4):

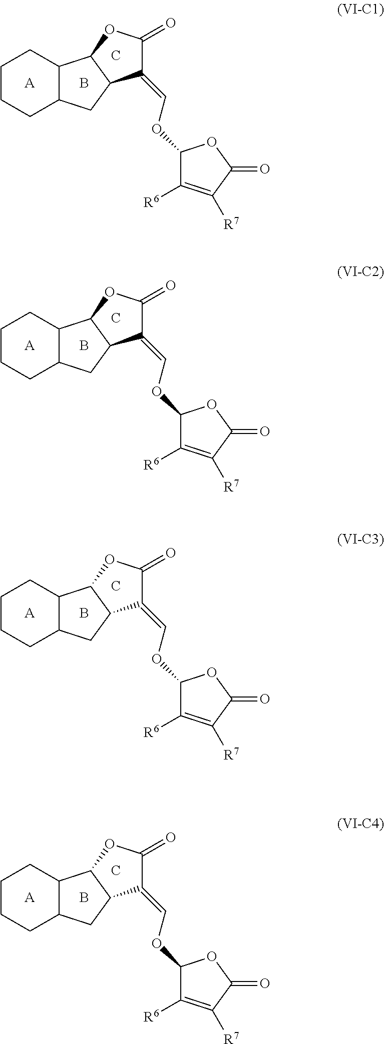

Unless otherwise indicated, reference to a molecular structure without identification of three-dimensional configuration, as in structures (V) or (VI), is intended to include all combinations of diastereomeric and enantiomeric possibilities. However, it should be emphasized that most, but not necessarily all, of the preferred terpenoid lactones herein are those isomers possessing the same stereochemistry as that of the natural strigolactones at the two adjacent chiral centers between rings B and C, exemplified by configurations (V-C1), (V-C2), (VI-C1), and (VI-C2) above.

Certain terpenoid lactones described herein and useful in conjunction with the methods and products of the invention are new chemical entities and accordingly claimed as such herein. In one embodiment, then, the invention provides a novel terpenoid lactone having the structure of formula (I)

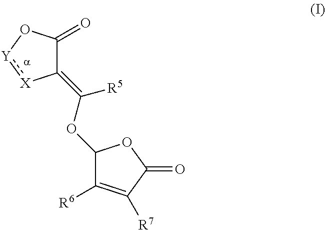

wherein α, R¹, R², R³, R⁴, R⁵, R⁸, and R⁹ are as defined above, and R⁶ and R⁷ are linked to form a C₅-C₁₄ cyclic group, which is optionally substituted with one or more nonhydrogen substituents and may contain one or more heteroatoms generally selected from N, O, and S. Such compounds may be represented by the structure of formula (VII)

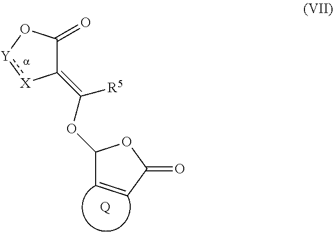

in which Q represents the optionally substituted, optionally heteroatom-containing C₅-C₁₄ cyclic group.

In this embodiment, the C₅-C₁₄ cyclic group may be either monocyclic or bicyclic; if bicyclic, the two rings may be linked or fused and identical or different. The cyclic group may be aromatic or alicyclic, or, if bicyclic, may comprise a combination of one aromatic ring and one alicyclic ring linked or fused together. If nonhydrogen substituents are present on the C₅-C₁₄ cyclic group, there are in the range of one to four substituents per ring, usually one or two substituents per ring. Any nonhydrogen substituents present on the cyclic group are selected from those functional groups and hydrocarbyl moieties set forth under the definition of “substituted” in part (I) of this section. Examples of C₅-C₁₄ cyclic groups thus include, without limitation, cyclopentadienyl, cyclohexenyl, phenyl, 1-methylphenyl, 2-methylphenyl, 2,3-dimethylphenyl, 1-ethylphenyl, 2-ethylphenyl, 2,3 diethylphenyl, 1-methoxyphenyl, 2-methoxyphenyl, 2,3-dimethoxyphenyl, 1-chlorophenyl, 2-chlorophenyl, 2,3-dichlorophenyl, 2-chloro-3-methylphenyl, 2,3-diethoxyphenyl, pyridinyl, naphthalenyl, and the like. Specific examples of such compounds are terpenoid lactones (4A) and (4B), the synthesis of which is described in Example 6.

The invention additionally provides a novel terpenoid lactone having the structure of formula (I) wherein α, R¹, R², R³, R⁴, R⁵, R⁸, and R⁹ are as defined previously, and wherein R⁶ and R⁷ are not linked to form a cyclic group as they are in the novel compounds just defined. Rather, in this embodiment, R⁶ is hydrogen and R⁷ is selected from halo, hydroxy, C₂-C₁₂ alkoxy, C₂-C₁₂ hydrocarbyl, substituted C₂-C₁₂ hydrocarbyl, heteroatom-containing C₂-C₁₂ hydrocarbyl, and substituted heteroatom-containing C₂-C₁₂ hydrocarbyl. In a generally preferred subset of such compounds, R⁷ is an optionally substituted, optionally heteroatom-containing C₂-C₁₂ hydrocarbyl moiety, more preferably an optionally substituted, optionally heteroatom-containing C₂-C₆ hydrocarbyl moiety. If heteroatoms are present there are generally not more than three, and they are typically selected from N, O, and S. Any nonhydrogen substituents present are generally selected from the functional groups set forth under the definition of “substituted” in part (I) of this section. Typical substituents include, without limitation, halo, hydroxy, lower alkoxy, and lower acyloxy. The R⁷ group may, however, be an unsubstituted C₂-C₁₂ hydrocarbyl moiety, in which case, again, preferred such moieties are C₂-C₆, and thus include, for example, ethyl, ethenyl, n-propyl, n-propenyl, isopropyl, isopropenyl, n-butyl, isobutyl, t-butyl, n-pentyl, cyclopentyl, cyclohexyl, and the like. Specific examples of such compounds are terpenoid lactones (1A) and (1B), synthesized as described in Example 2.

In a related embodiment, the invention provides a novel terpenoid lactone having the structure of formula (I) wherein, as with the novel terpenoid lactones just described, a, R¹, R², R³, R⁴, R⁵, R⁸, and R⁹ are as defined previously, and R⁶ and R⁷ are not linked to form a cyclic group. In this embodiment, however, R⁶ is a nonhydrogen substituent, and R⁷ may or may not be a nonhydrogen substituent. More specifically, R⁶ is selected from halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, and R⁷ is selected from hydrogen, halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl. In a generally preferred subset of such compounds, R⁷ is other than hydrogen, such that the “lower” lactone ring of the molecular structure has a substituent other than hydrogen on each carbon atom of the lactone's double bond. Preferably, although not necessarily, R⁶ and R⁷ are both optionally substituted, optionally heteroatom-containing C₁-C₁₂ hydrocarbyl moieties, e.g., optionally substituted, optionally heteroatom-containing C₁-C₁₂ alkyl moieties, including “lower” such moieties that are C₁-C₆, and although R⁶ and R⁷ may be the same or different, it is generally the case that R⁶ and R⁷ are the same. As before, if heteroatoms are present there are generally not more than three, and they are typically selected from N, O, and S; any nonhydrogen substituents on the C₁-C₁₂ hydrocarbyl moieties are selected from the functional groups set forth under the definition of “substituted” in part (I) of this section, Typical substituents include, without limitation, halo, hydroxy, lower alkoxy, and lower acyloxy. In a particularly preferred subset of these terpenoid lactones, R⁶ and R⁷ are optionally substituted, optionally heteroatom-containing C₁-C₆ hydrocarbyl moieties. Examples of unsubstituted such moieties that may serve as R⁶ and/or R⁷ in this embodiment include methyl, ethyl, ethenyl, n-propyl, n-propenyl, isopropyl, isopropenyl, n-butyl, isobutyl, t-butyl, n-pentyl, cyclopentyl, cyclohexyl, and the like. Specific examples of such compounds are terpenoid lactones (3A) and (3B), synthesized as described in Example 7.

In a further embodiment, novel terpenoid lactones are provided having the structure of formula (VIII)

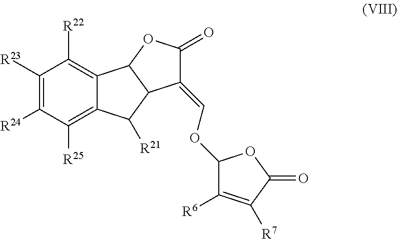

wherein:

R⁶ and R⁷ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, or R⁶ and R⁷ may be taken together to form a C₅-C₁₄ cyclic group, optionally substituted and/or containing at least one heteroatom;

R²¹ is selected from hydrogen, hydroxy, C₁-C₃ alkoxy, and C₂-C₄ acyloxy; and either

(a) one of R²², R²³, R²⁴, and R²⁵ is C₁-C₁₂ hydrocarbyl, optionally substituted and optionally heteroatom-containing, and the others are hydrogen; or

(b) R²², R²³, R²⁴, and R²⁵ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, substituted C₁-C₁₂ hydrocarbyl, heteroatom-containing C₁-C₁₂ hydrocarbyl, and substituted heteroatom-containing C₁-C₁₂ hydrocarbyl, with the proviso that at least one of R²², R²³, R²⁴, and R²⁵ is optionally substituted, optionally heteroatom-containing C₁-C₁₂ hydrocarbyloxy.

The R⁶ and R⁷ substituents may be any of a number of moieties, including, but not limited to, those set forth with respect to the novel terpenoid lactones described above, but will, in a particularly preferred embodiment, be identical to the substituents present in naturally occurring strigolactone, such that R⁶ is hydrogen and R⁷ is methyl. R²¹, as noted, may be any of hydrogen, hydroxy, C₁-C₃ alkoxy, and C₂-C₄ acyloxy, but is typically hydrogen.

Then, in compounds defined by (a), one of the substituents on the aromatic “A” ring is a nonhydrogen substituent, while the other substituents on the ring are hydrogen atoms. The nonhydrogen substituent is an optionally substituted and/or heteroatom-containing C₁-C₁₂ hydrocarbyl group, which generally, although not necessarily, is an optionally substituted and/or heteroatom-containing C₁-C₈ hydrocarbyl group, including substituted and unsubstituted C₁-C₈ alkyl groups, with unsubstituted such groups exemplified by the C₁-C₆ alkyl groups methyl, ethyl, n-propyl, isopropyl, n-butyl, isobutyl, t-butyl, cyclopentyl, cyclohexyl, and the like. Specific examples of such compounds include, without limitation, compounds (5A) and (5B), synthesized in Example 3. In compounds defined by (b), it should be noted that one of the aromatic substituents is an optionally substituted, optionally heteroatom-containing C₁-C₁₂ hydrocarbyloxy group, i.e., an optionally substituted and/or heteroatom-containing O—R group where R is hydrocarbyl as defined in part (I) of this section. Preferred C₁-C₁₂ hydrocarbyloxy groups are C₁-C₁₂ alkoxy, with unsubstituted C₁-C₈ alkoxy, especially C₁-C₆ alkoxy, being particularly preferred. Exemplary such compounds include compounds (6A), (6B), (7A), (7B), (8A), and (8C), synthesized in Examples 1, 4, and 5.

Specific terpenoid lactones useful in conjunction with the present methods and compositions are the strigolactones below:

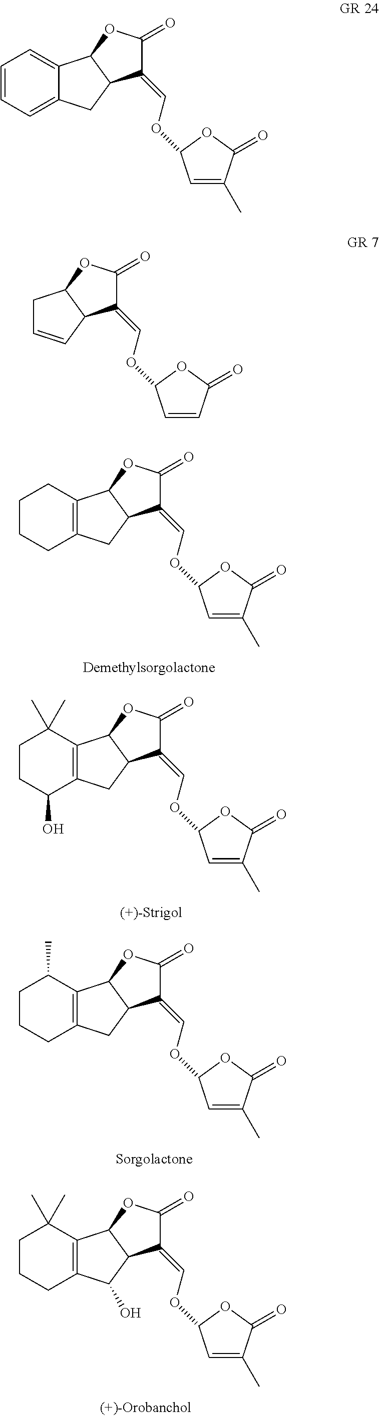

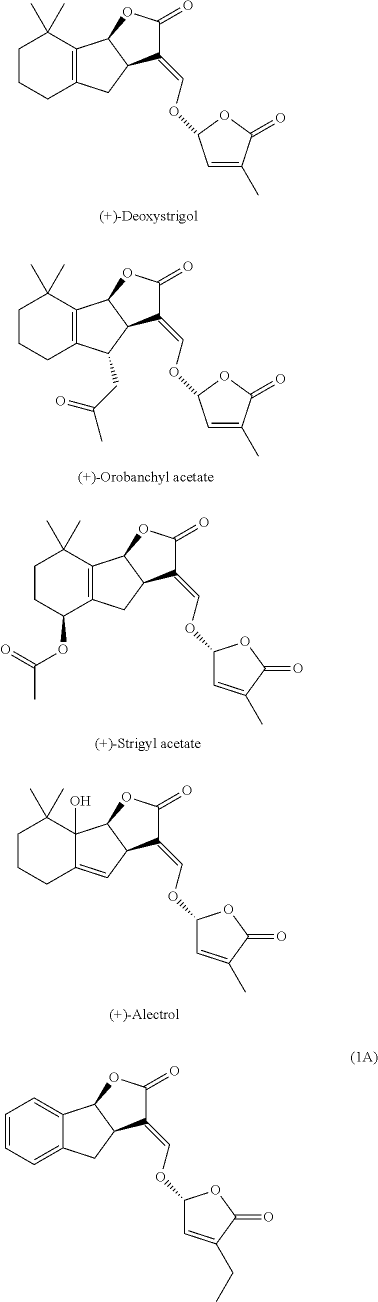

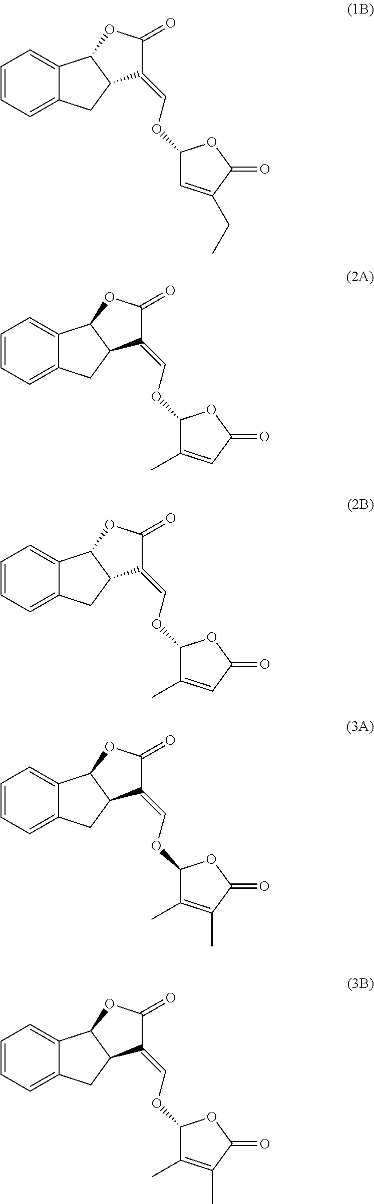

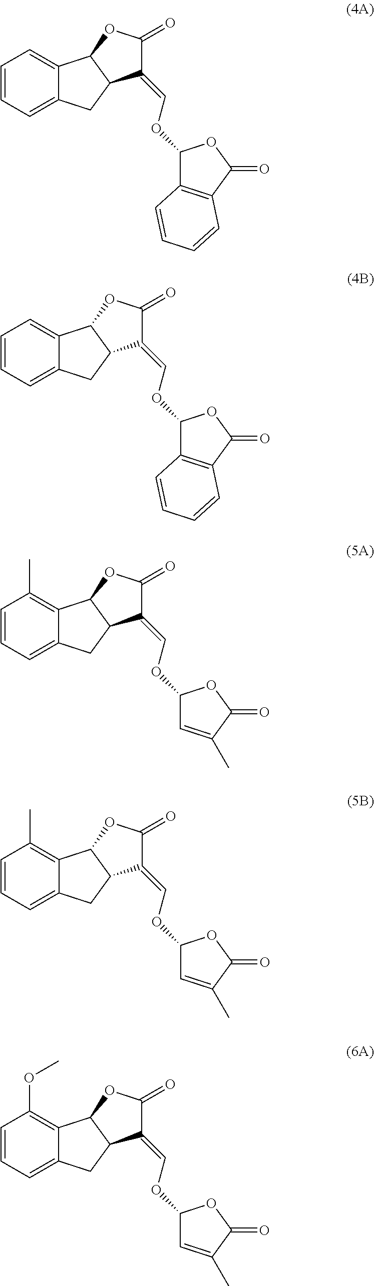 [SMILES: O=C1O[C@@H]2c3ccccc3C[C@@H]2/C1=C\O[C@@H]1OC(=O)c2ccccc21 ; O=C1O[C@H]2c3ccccc3C[C@H]2/C1=C\O[C@@H]1OC(=O)c2ccccc21 ; CC1=C[C@H](O/C=C2/C(=O)O[C@@H]3c4c(C)cccc4C[C@H]23)OC1=O ; CC1=C[C@H](O/C=C2/C(=O)O[C@H]3c4c(C)cccc4C[C@@H]23)OC1=O ; COc1cccc2c1[C@H]1OC(=O)/C(=C/O[C@H]3C=C(C)C(=O)O3)[C@H]1C2]

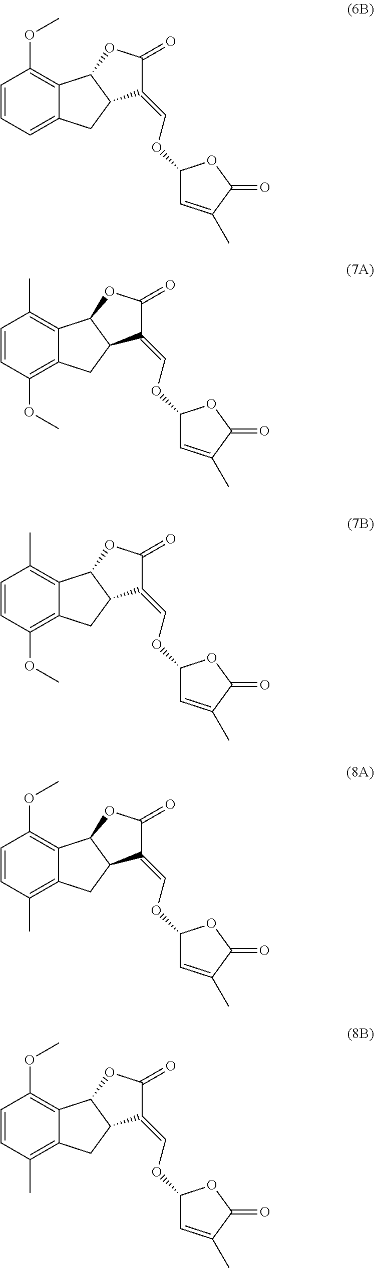 [SMILES: COc1cccc2c1[C@@H]1OC(=O)/C(=C/O[C@H]3C=C(C)C(=O)O3)[C@@H]1C2 ; COc1ccc(C)c2c1C[C@@H]1/C(=C\O[C@H]3C=C(C)C(=O)O3)C(=O)O[C@H]21 ; COc1ccc(C)c2c1C[C@H]1/C(=C\O[C@H]3C=C(C)C(=O)O3)C(=O)O[C@@H]21 ; COc1ccc(C)c2c1[C@H]1OC(=O)/C(=C/O[C@H]3C=C(C)C(=O)O3)[C@H]1C2 ; COc1ccc(C)c2c1[C@@H]1OC(=O)/C(=C/O[C@H]3C=C(C)C(=O)O3)[C@@H]1C2]

It will be appreciated that other terpenoid lactones, and strigolactone analogs in particular, may be synthesized by modification of naturally occurring compounds, by modification of known synthetic compounds, by using techniques analogous to those set forth in Examples 1 through 7 herein, and/or by using synthetic methods known to those of ordinary skill in the art of synthetic organic chemistry and/or described in the pertinent texts and literature. See, e.g., Thuring et al. (1997) J. Agric. Food Chem. 45:507-513; Nefkens et al. (1997) J. Agric. Food Chem. 45:2273-77; Kendall et al, (1979) J. Org. Chem. 44(9) 1421-24; Sugimoto et al. (1259) J. Org. Chem. 63:1259-67; Kadas et al. (1994) Tetrahedron 50(9):2895-2906; Thuring et al. (1994) Tetrahedron 51(17):5047-56; Malik et al. (2010) Tetrahedron 66:7198-7203; Sugimoto et al. (1997) Tet. Lett, 38(13):2321-24; Zwanenburg et al. (1997) Pure & Appl. Chem. 69(3):651-4; Mwakaboko et al. (2011) Plant Cell Physiol. 52(4):699-715; and Howie et al. (1976) J. Med. Chem. 19(2):309-13. Any such compound that is currently known or that is discovered or invented hereinafter is considered to be within the scope of the invention and thus suitable for use as the terpenoid lactone component of the present compositions.

The pharmaceutical compositions containing the terpenoid lactone that is a selective activator of SIRT1 are unit dosage forms that typically contains about 0.01 mg to about 1 g of the compound, generally about 0.01 mg to about 500 mg, more usually about 0.01 mg to about 250 mg, more typically about 0.05 mg to about 100 mg, still more typically about 0.05 mg to about 75 mg, and optimally about 0.05 mg to about 50 mg of the compound. Examples of unit dosages thus include, without limitation, 0.01 mg, 0.05 mg, 0.10 mg, 0.25 mg, 0.50 mg, 1.0 mg, 2.5 mg, 5.0 mg, 10.0 mg, 25.0 mg, 50.0 mg, 100 mg, 250 mg, 500 mg, and 1 g. These unit dosages generally represent unit dosages for once daily or twice daily oral administration.

In another embodiment, a composition is provided that contains a combination of a terpenoid lactone as just described, i.e., a terpenoid lactone that is a selective activator of SIRT1, and an additional SIRT1 activator that may or may not be a selective activator of SIRT1. The latter compound, like the selective activator of SIRT1, is a compound that measurably increases the activity of SIRT1 in a cell, particularly a eukaryotic cell, and/or in the body. Like the selective SIRT1 activator, the additional SIRT1 activator increases the level of the SIRT1 protein and/or increases at least one activity of sum by at least about 10%, 25%, 50%, or more. Any compound or composition of matter that increases the activity of SIRT1 in the body may be used as the additional SIRT1 activator, including known SIRT1 activators as well as those that are yet to be discovered or invented. Examples of additional SIRT1 activators that can be combined with the terpenoid lactone are compounds within the general structurally recognized classes of stilbenoids, flavonoids, chalconoids, tannins, and nicotinamide inhibition antagonists.

Stilbenoids, as is well known, are hydroxylated derivatives of stilbene, and are generally hydroxylated trans stilbenes having the structure of formula (IX)

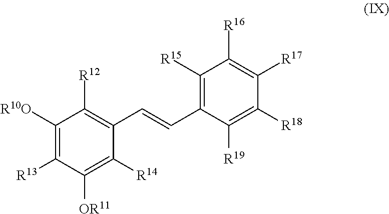

wherein:

R¹⁰ is selected from hydrogen, C₁-C₆ alkyl, halogenated C₁-C₆ alkyl, C₂-C₆ acyl, and a glycoside;

R¹¹ is selected from hydrogen, C₁-C₆ alkyl, halogenated C₁-C₆ alkyl, and C₂-C₆ acyl;

R¹², R¹⁴, R¹⁵, and R¹⁹ are independently selected from hydrogen, halo, C₁-C₆ alkyl, and halogenated C₁-C₆ alkyl; and

R¹³, R¹⁶, R¹⁷, and R¹⁸ are independently selected from hydrogen and OR²⁰, where R²⁰ is hydrogen, C₁-C₆ alkyl, halogenated C₁-C₆ alkyl, or C₂-C₆ acyl;

or is an oligomer or glycoside thereof.

In a preferred embodiment, R¹², R¹⁴, R¹⁵, and R¹⁹ are hydrogen, and R¹⁰ and R¹¹ are independently selected from hydrogen and C₁-C₆ alkyl. For instance, R¹⁰ and R¹¹ may both be hydrogen, or they may both be methyl. R²⁰ is typically hydrogen or C₁-C₆ alkyl,

Specific stilbenoids useful in conjunction with the invention include, by way of example, resveratrol (3,5,4″-trans-trihydroxystilbene), pinosylvin (3,5-trans-dihydroxystilbene), and piceatannol (3′,4′,3,5-tetrahydroxy-trans-stilbene); the oligomeric stilbenoids alpha-viniferin, epsilon-viniferin, ampelopsin A, ampelopsin E, flexuosol A, gnetin H, hernsleyanol D, hopeaphenoi, and trans-diptoindoesin B; and the stilbenoid glycosides astringin and piceid.

Flavonoids useful herein include flavanols, flavonols, flavones, isoflavones, and anthocyanins.

The flavanols useful herein are generally the flavan-3-ols, which are flavonoids having the 3,4-dihydro-2H-chromen-3-ol skeleton

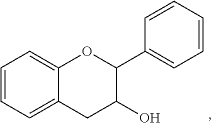 [SMILES: OC1Cc2ccccc2OC1c1ccccc1]

which include the catechins and other compounds found in green tea. Examples of preferred flavanols include catechin, epicatechin, epigallocatechin, epicatechin gallate, epigallocatechin gallate, epiafzelechin, fisetinidol, guibourtinidol, mesquitol, and robinetiniclol.

Flavonols, by contrast, are hydroxylated ketones, i.e., flavonoids that have the 3-hydroxyflavone backbone

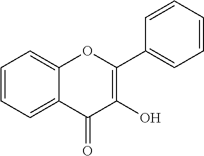 [SMILES: O=c1c(O)c(-c2ccccc2)oc2ccccc12]

and include, for instance, 3-hydroxyflavone, azaleatin, fisetin, galangin, gossypetin, kaempferide, kaempferol, isorhamnetin, morin, myricetin, natsudaidain, pachypodol, quercetin, rhamnazin, and rhamnetin. Flavonol glycosides are also suitable SIRT1 activators.

Flavones have the core structure

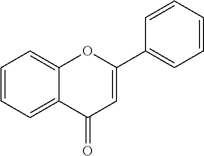 [SMILES: O=c1cc(-c2ccccc2)oc2ccccc12]

and include compounds such as apigenin, luteolin, tangeritin, chrysin, 6-hydroxyflavone, baicalein, scutellarein, wogonin, diosmin, and flavoxate.

Isoflavones having the core structure

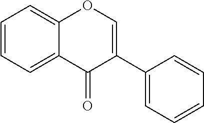 [SMILES: O=c1c(-c2ccccc2)coc2ccccc12]

and representative such compounds include genistein (4′5,7-trihydroxyisoflavone) and daidzein (4,7-dihydroxyisoflavone).

The anthocyanins include compounds such as aurantinidin, cyanidin, delphinidin, europinidin, luteolinidin, malvidin, pelargonidin, peonidin, petunidin, and rosinidin, as well as anthocyanidins, which are glycosides (usually the 3-glucosides) of the aforementioned anthocyanins. These are cationic tricyclic compounds having the general structure

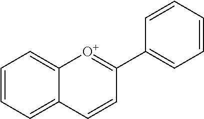 [SMILES: c1ccc(-c2ccc3ccccc3[o+]2)cc1]

where the rings are substituted with one or more hydroxyl and/or methoxy groups.

Chalconoids are compounds that have the structural backbone of chalcone

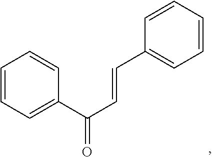 [SMILES: O=C(/C=C/c1ccccc1)c1ccccc1]

and include compounds such as butein (2′,3,4,4′-tetrahydroxychalcone) and isoliquiritigenin (2,4,4′-trihydroxychalcone).

Tannins suitable as SRT1 activators herein include phiorotannins such as phloroglucinol, hydrolysable tannins such as gallic acid and gallic acid derivatives, and non-hydrolyzable tannins, particularly flavones and derivatives thereof.

Nicotinamide inhibition antagonists herein are compounds that compete with nicotinamide to facilitate the deacetylation activity of SIRT1, See, e.g., Yang et al. (2005) The AAPS Journal 8(4):E632-E643 (Article 72). A representative such compound is isonicotinamide.

Pharmaceutical compositions containing both the terpenoid lactone and the additional SIRT1 activator will generally include the compounds in a weight ratio of about 1:100 to 100:1, more typically in the range of about 1:10 to about 10:1, and most typically in the range of about 1:5 to about 5:1, including, for instance, weight ratios of the terpenoid lactone to the additional SIRT1 activator of about 1:75, 1:50, 1:25, 1:15, 1:10, 1:5, 1:2.5, 1:1, 2.5:1, 5:1, 10:1, 15:1, 25:1, 50:1, and 75:1. In orally administrable compositions, a unit dosage form typically contains about 10 mg to 1 g, preferably about 25 mg to about 500 mg, and optimally about 40 mg to about 400 mg, of each of the additional SIRT1 activator (e.g., resveratrol) and the terpenoid lactone.

### III. Methods of Use:

In one embodiment, the aforementioned terpenoid lactone or combination of a terpenoid lactone with an additional SIRT1 activator, e.g., a stilbenoid such as resveratrol or pinosylvin, is used to influence energy metabolism in a eukaryotic cell, in a method that involves contacting the cell with the terpenoid lactone and optionally the additional SIRT1 activator in amounts effective to influence energy metabolism. The manner in which energy metabolism of the eukaryotic cell is influenced may be any modification of one or more biochemical reactions involved in energy changes. The modification of one or more biochemical reactions will generally involve an increase or decrease in the availability of a reactant, enzyme, substrate, or co-substrate, an increase or decrease in a naturally occurring reaction inhibition process, an inhibition of the activity of a particular enzyme, an increase or decrease of a particular reaction product, an increase or decrease in the rate of a reaction, etc. The cell may be contacted with the terpenoid lactone and optionally the additional SIRT1 activator separately, either simultaneously or sequentially, although more typically, the cell is contacted with the terpenoid lactone and the additional SIRT1 activator simultaneously with both compounds in a single composition, in the event that an additional SIRT1 activator is employed. It will be understood that a eukaryotic cell is any cell found in a eukaryotic organism, including fungi, protozoa, and animals, e.g., humans, ovines, bovines, equines, porcines, canines, felines, non-human primates, mice, rats, and the like. The amount of each compound used to influence the energy metabolism of a cell or group of cells can be determined experimentally using, for example, the methods described in the examples herein.

In a preferred embodiment, a method is provided for influencing energy metabolism in a eukaryotic cell as just described but wherein the cell is contacted with the terpenoid lactone, e.g., strigolactone or a strigolactone analog, without also be contacted by an additional SIRT1 activator.

In another embodiment, the ability of a terpenoid lactone that is a selective SIRT1 activator, and optionally an additional SIRT1 activator, to influence energy metabolism in a eukaryotic cell is implemented in the context of a method for treating a subject suffering from or predisposed to develop a metabolic disorder. In this embodiment, a therapeutically effective amount of a terpenoid lactone and optionally a therapeutically effective amount of an additional SIRT1 activator are administered to the subject. The terpenoid lactone and the optional additional SIRT1 activator may be administered simultaneously, either separately or, more preferably, in a single pharmaceutical formulation, or the compounds may be administered sequentially, at different times or according to a different dosage regimen. In a preferred embodiment, the terpenoid lactone is administered in the absence of any additional SIRT1 activators.

The metabolic disorder may be type 2 diabetes or obesity, or Metabolic Syndrome or any one or more of the conditions associated with Metabolic Syndrome, including, without limitation, hypertension, insulin resistance, and dyslipidemia. The metabolic disorder may also involve various aspects of the aging process as well as adverse skin conditions, particularly those adverse skin conditions associated with aging. Other disorders that can be treated with the present compositions include cardiovascular disease, neurological disorders, inflammatory conditions, and other diseases, disorders, and adverse conditions that can be alleviated, cured, or prevented by virtue of influencing energy metabolism in eukaryotic cells, increasing mitochondrial activity, and/or slowing the aging process of an organism. As the compositions and methods of the invention have utility in contexts where aging plays a role, they also have utility in preventing or treating aging-related conditions, including aging-related skin conditions such as hyperpigmentation, wrinkles, sun damage, skin discoloration, and the like, as well as aging-related ophthalmic disorders such as dry eye syndrome, cataracts, yellowing of the lens, loss of night vision, etc.

Cardiovascular diseases that can be treated using the compositions and methods of the invention include, by way of example, cardiomyopathy, such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using the methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Still other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The methodology also extends to the prevention or treatment of restenosis following coronary intervention.

Neurological disorders that can be treated using the compositions and methods of the invention include, without limitation, Alzheimer's Disease, aphasia, Bell's Palsy, Creutzfeldt-Jakob Disease, encephalitis, epilepsy, Huntington's Disease, Parkinson's Disease, Tardive Dyskinesia, Amyotrophic Lateral Sclerosis, Guillain-Barre Syndrome, Muscular Dystrophy, Multiple Sclerosis, and Meniere's Disease.

Inflammatory conditions that can be treated using the compositions and methods of the invention include, for example, rheumatism, osteoarthritis, gastrointestinal inflammatory disorders, SLE and other autoimmune disorders, and the like.

### IV. Pharmaceutical Formulations and Modes of Administration:

As discussed in the preceding section, the invention provides methods for treating any condition, disease or disorder that is responsive to the administration of a terpenoid lactone that is a selective activator of SIRT1, alone or in combination with an additional SIRT1 activator, wherein those conditions, diseases and disorders are generally metabolic disorders, i.e., associated with cellular energy metabolism, and/or are related to aging. The compounds and compositions can be administered to a subject by themselves or in pharmaceutical formulations in which they are mixed with suitable pharmaceutically acceptable carriers, also referred to in the art as excipients. When the terpenoid lactone is administered with the additional SIRT1 activator, the two compounds may be administered separately, in different dosage forms, or simultaneously, either in one dosage form or in two different dosage forms.

Pharmaceutical formulations suitable for use in conjunction with the present invention include compositions wherein the active agent is contained in a “therapeutically effective” amount, i.e., in an amount effective to achieve its intended purpose. Determination of a therapeutically effective amount for any particular terpenoid lactone and for any particular SIRT1 activator is within the capability of those skilled in the art. Generally, toxicity and therapeutic efficacy of a compound or composition described herein can be determined by standard pharmaceutical procedures in cell cultures or experimental animals, e.g., procedures used for determining the maximum tolerated dose (MTD), the ED₅₀, which is the effective dose to achieve 50% of maximal response, and the therapeutic index (TI), which is the ratio of the MTD to the ED₅₀. Obviously, compounds and compositions with high TIs are the more preferred compounds and compositions herein, and preferred dosage regimens are those that maintain plasma levels of the active agents at or above a minimum concentration to maintain the desired therapeutic effect. Dosage will, of course, also depend on a number of factors, including the particular compound or composition, the site of intended delivery, the route of administration, and other pertinent factors known to the prescribing physician.

Administration of a compound or composition of the invention may be carried out using any appropriate mode of administration. Thus, administration can be, for example, oral, parenteral, transdermal, transmucosal (including rectal and vaginal), sublingual, by inhalation, or via an implanted reservoir in a dosage form. The term “parenteral” as used herein is intended to include subcutaneous, intravenous, and intramuscular injection.

Depending on the intended mode of administration, the pharmaceutical formulation containing the terpenoid lactone and optionally an additional SIRT1 activator may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). For those compounds that are orally active, oral dosage forms are generally preferred, and include tablets, capsules, caplets, solutions, suspensions and syrups, and may also comprise a plurality of granules, beads, powders, or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms for those terpenoid lactones and SIRT1 activators that are orally active, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The compounds and compositions of the invention may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition, the compounds may also be formulated as a depot preparation for controlled release of the active agent, preferably sustained release over an extended time period. These sustained release dosage forms are generally administered by implantation (e.g., subcutaneously or intramuscularly or by intramuscular injection).

Although the present compositions will generally be administered orally, parenterally, transdermally, or via an implanted depot, other modes of administration are suitable as well. For example, administration may be rectal or vaginal, preferably using a suppository that contains, in addition to the active agent, excipients such as a suppository wax. Formulations for nasal or sublingual administration are also prepared with standard excipients well known in the art. The pharmaceutical compositions of the invention may also be formulated for inhalation, e.g., as a solution in saline, as a dry powder, or as an aerosol.

### EXAMPLES

Examples 1-7 describe synthesis of novel terpenoid lactones useful in conjunction with the compounds and compositions of the invention.

The lactone reactants used in Examples 1-7 that were not obtained commercially were synthesized as follows:

Synthesis of 5-methoxy-8-methyl-3,3a,4,8b-tetrahydro-indeno[1,2-b]furan-2-one (10d)

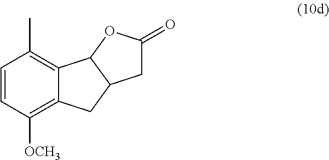 [SMILES: COc1ccc(C)c2c1CC1CC(=O)OC21]

was carried out as described in steps (a) through (d), below.

(a) 2-Ethoxycarbanylmethyl-4-methoxy-7-methyl-1-oxo-indan-2-carboxylic acid ethyl ester was synthesized according to Scheme 1:

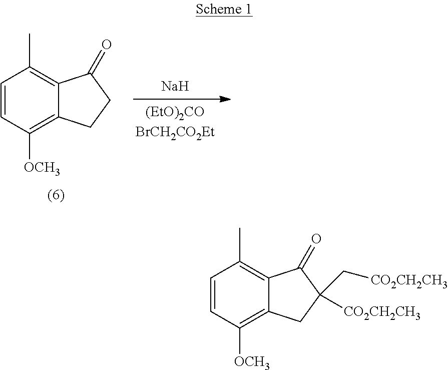

A solution of 4-methoxy-7-methyl-indan-1-one (5 g, 28.4 mmol) in DMF (15 mL) was added slowly into a solution of diethyl carbonate (13.8 mL, 114 mmol) and sodium hydride (2.50 g, 62.5 mmol) in DMF (40 mL) with stirring at 0° C. The reaction mixture was allowed to warm to room temperature, and stirring was continued for 10 minutes. The temperature of the reaction mixture was then raised to 65° C., and stirring was continued for 1 hr. At that point, ethyl bromoacetate (4.73 mL, 42.6 mmol) was added dropwise to the mixture. The resulting mixture was heated at 65° C. for another 1 hr, at which point LC-MS showed the reaction to be finished. The solution was neutralized with glacial acetic acid, and then diluted with EtOAc/Hex (3/1) and H₂O. The organic layer was extracted, dried and concentrated. The crude mixture was purified by flash column chromatography (AcOEt:Hexanes 3:7) to afford compound (6) (10 g, 100% yield), LCMS (ESI): m/z 335 (M+H)⁺.

(b) Conversion to (7) was carried out according to Scheme 2:

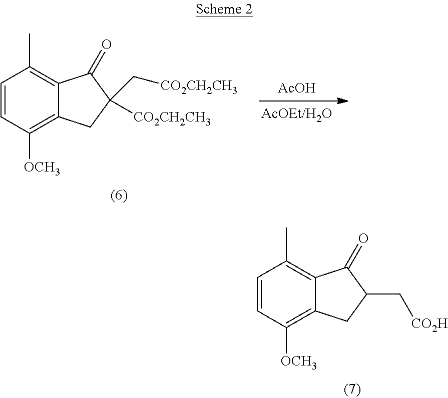

A solution of (6) (10 g, 28.5 mmol) in AcOH (13 in L) and HCl (6N, 13 mL) was stirred at 110° C. overnight. After the reaction was cooled down, the mixture was diluted with AcOEt and H₂O. The organic layer was extracted, dried and concentrated. The crude mixture was triturated with Et₂O to afford the pure compound (7) (4.5 g, 64% yield) as a white powder.

LCMS (ESI): m/z 235 (M+H)⁺

(c) Conversion to (8) was carried out according to Scheme 3:

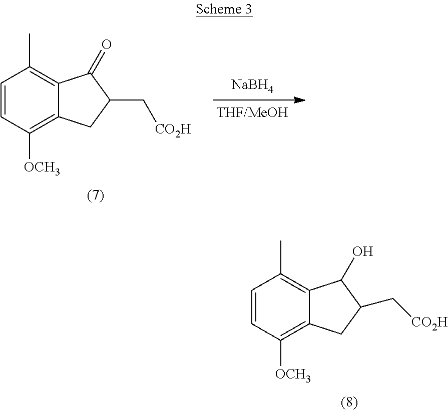 [SMILES: COc1ccc(C)c2c1CC(CC(=O)O)C2=O ; "NaBH4" ; COc1ccc(C)c2c1CC(CC(=O)O)C2O]

To a solution of (7) (4.5 g, 19.2 mmol) in MeOH (40 mL) and THF (45 mL) was added NaBH₄ (2.92 g, 76.8 mmol) at 0° C. in portions. After the mixture was stirred at room temperature for 2 hrs, HCl (6N) was used to adjust the solution to acidic. The solvent was removed in vacuo. The crude mixture was diluted with AcOEt and H₂O. The organic layer was extracted, washed with brine, dried and concentrated to give the crude compound (8) (4.0 g), which was directly used in the next step without purification. LCMS (ESI): 235 (M−H)⁻.

(d) Synthesis of (10d) from (8) was carried out according to Scheme 4:

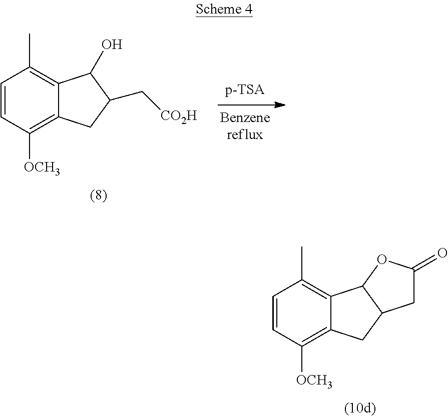 [SMILES: COc1ccc(C)c2c1CC(CC(=O)O)C2O ; "p-TSA" ; COc1ccc(C)c2c1CC1CC(=O)OC21]

To a solution of acid alcohol 8 (crude, 4.0 g) in benzene (50 mL) was added pTSA (250 mg). The resulting solution was stirred at 65° C. for 2 hrs. The solvent was removed in vacuo. The crude mixture was diluted with AcOEt and H₂O. The organic layer was extracted, washed with brine, dried and concentrated. The crude mixture was purified by flash column chromatography (AcOEt: Hexanes/3:7) to afford the compound 10d (3.5 g, 84% yield in two steps).

LCMS (ESI): m/z 219 (M+H)⁺. ¹H NMR (300 MHz, CDCl₃): 7.02 (d, 1H), 6.78 (d, 1H), 5.92 (d, 1H), 3.81 (s, 3H), 3.37 (m, 1H), 3.21 (dd, 1H), 2.92 (dd, 1H), 2.82 (dd, 1H), 2.42 (dd, 1H), 2.38 (s, 3H).

The following lactones were prepared in an analogous manner:

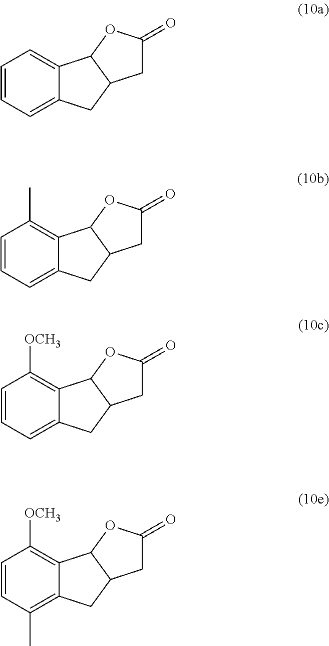 [SMILES: O=C1CC2Cc3ccccc3C2O1 ; Cc1cccc2c1C1OC(=O)CC1C2 ; COc1cccc2c1C1OC(=O)CC1C2 ; COc1ccc(C)c2c1C1OC(=O)CC1C2]

In synthesizing lactones (10a), (10b), (10c), and (10e), the following reactants were respectively substituted for the 4-methoxy-7-methyl-indan-1-one used in the synthesis of (10d): indan-1-one; 7-methyl-indan-1-one; 7-methoxy-indan-1-ione; and 4-methyl-7-methoxy-indan-1-one.

Lactone (10a): LCMS (ESI): m/z 175 (M+H)⁺. ¹H NMR (300 MHz, CDCl₃) δ 7.47 (d, 1H), 7.38-7.25 (m, 3H), 5.90 (d, 1H), 338 (m, 2H), 2.90 (m, 2H), 2.41 (dd, 1H).

Lactone (10b): LCMS (ESI): m/z 189 (M+H)⁺, ¹H NMR (300 MHz, CDCl₃) δ 7.26 (m, 1H), 7.08 (m, 2H), 5.98 (d, 1H), 3.38 (m, 2H), 2.90 (m, 2H), 2.42 (dd, 1H), 2.41 (s, 3H).

Lactone (10e): LCMS (ESI): m/z 205 (M+H)⁺. ¹H NMR (300 MHz, CDCl₃) δ 7.34 (dd, 1H), 6.82 (d, 1H), 6.78 (d, 114), 6.00 (d, 1H), 3.84 (s, 3H), 3.36 (m, 2H), 2.90 (m, 2H), 2.46 (dd, 1H).

Lactone (10e): LCMS (ESI): m/z 219 (M+II)⁺. ¹H NMR (300 MHz, CDCl₃) δ 7.10 (d, 1H), 6.64 (d, 1H), 6.00 (d, 1H), 3.82 (s, 3H), 3.32 (m, 2H), 2.94 (dd, 1H), 2.78 (chi, 1H), 2.48 (dd, 1H), 2.20 (s, 3H).

### Example 1

The terpenoid lactones having the molecular structure of formula (7A) and (7B) were synthesized as described below.

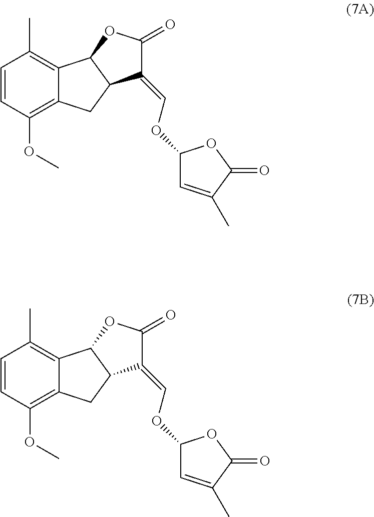 [SMILES: COc1ccc(C)c2c1C[C@@H]1/C(=C\O[C@H]3C=C(C)C(=O)O3)C(=O)O[C@H]21 ; COc1ccc(C)c2c1C[C@H]1/C(=C\O[C@H]3C=C(C)C(=O)O3)C(=O)O[C@@H]21]

Step 1,1. Preparation of 2-methyl-4-oxa-tricyclo[5.2.1.0^{2,6}]dec-8-ene-3,5-dione (1):

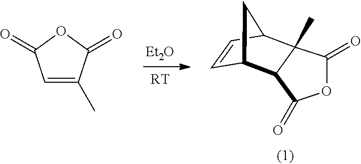

A flask (500 mL), equipped with a Vigreux column (20 cm) and a distillation system, was charged with dicyclopentadiene (150 mL) and heated to 210° C. Cyclopentadiene was recovered in an ice-cold flask. The cyclopentadiene was then added to a solution of 3-methyl-furan-2,5-dione (40 g, 0.36 mol) in ether (100 mL). The reaction mixture was stirred overnight (16 h) at room temperature. The solvent was removed to give a white solid. The solid was treated with hexanes to give the title compound 1 (60.5 g, 95%).

### Step 1.2. Preparation of 5-hydroxy-2-methyl-4-oxa-tricyclo[5.2.1.0^{2,6}]dec-8-en-2one ((2a) and (2b))

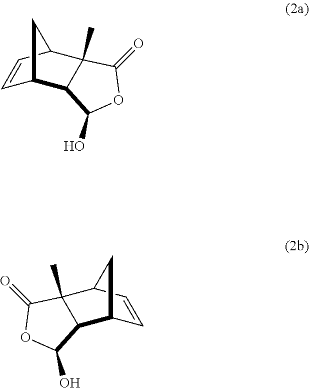

A solution of 2-methyl-4-oxa-tricyclo[5.2.1.0^{2,6}]dec-8-ene-3,5-dione (1, 5.34 g, 30 mmol) in TIN (100 mL) at −40° C. was added Li(t-BuO)₃AlH (9.2 g, 36 mmol) portion-wise over 30 minutes. The mixture was stirred at −20° C. for 5 h. To the solution was slowly added 2N HCl to pH ˜1. After removal of the solvent, the residue was extracted with EtOAc, and organics were washed with water and brine, then dried over Na₂SO₄. The solution was evaporated and the residue was purified by silica gel column to give 5-hydroxy-2-methyl-4-oxa-tricyclo[5.2.1.0^{2,6}]dec-8-en-3-one (a mixture of two enantiomers ((2a) and (2b)) as white foam (4.0 g, 75%).

Isolation of enantiomer (2a):

Step 1.3. A mixture of enantiomers (2a) and (2b) (4.0 g, 22.2 mmol), p-TsOH (0.21 g, 1.1 mmol) and 1-menthol (4.17 g, 26.7 mmol) in benzene (150 mL) in a flask fitted with a Dean-Stark trap was heated under reflux for 18 h. After evaporation of the solvent, the residue was dissolved in EtOAc (80 mL), washed with water, brine, and dried over Na₂SO₄. The solution was evaporated to give a crude product, which was crystallized from hexanes to give pure 1-mentholoxylactone 3 (2.0 g, 28%).

Step 1.4. 1-Mentholoxylactone (3) (2.0 g, 6.3 mmol) was then dissolved in 80% (v/v) TFA in water (20 mL), and the solution was stirred for 18 h at room temperature. After removal of the solvent under reduced pressure, the crude product was purified by chromatography to give (2a) (1.0 g, 88%) in enantiomerically pure form, as compound (4).

### Step 1.5. Preparation exo-chloro lactone (5)

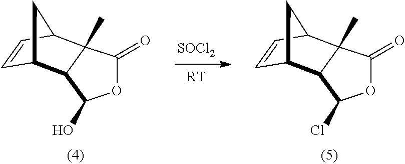

Enantiopure (4) (1.0 g, 5.6 mmol) was dissolved in SOCl₂ (10 in L) in the presence of pyridine (0.48 g, 6.1 mmol) at 0° C. The solution was allowed to warm up to room temperature and then stirred for 1 h. Excess SOCl₂ was removed. The residue was purified by chromatography to give exo-chloro lactone (5) (0.81 g, 73%).

### Step 1.6. Preparation of (9)

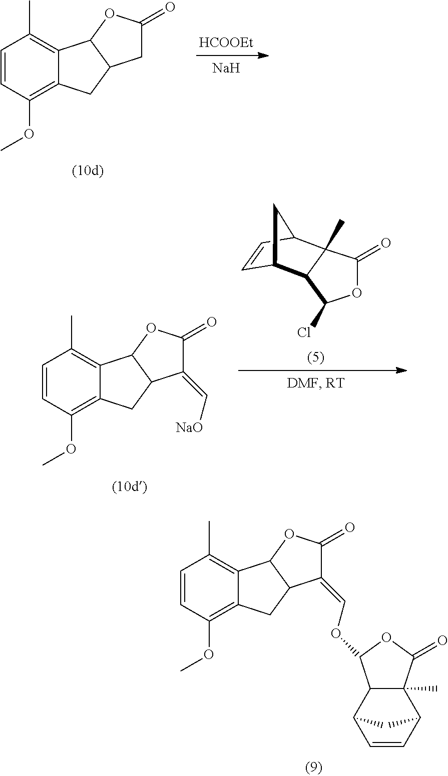

To a cooled (0° C.) and stirred solution of tricyclic lactone (10d) (654 mg, 3 mmol) in ethyl formate (20 mL) was added, under nitrogen, 1.2 eq NaH (144 mg, 3.6 mmol). The mixture was allowed to warm to room temperature and stirred for 3 h. When TLC analysis indicated the complete formylation excess ethyl formate was removed under reduced pressure. The sodium salt (10d′) obtained was dissolved in DMF (20 mL) and cooled to 0° C. Upon addition of exo-5-(5)-chlorolactone (5) (600 mg, 3 mmol), the mixture was stirred overnight. Then DMF was removed in vacuo to give a residue, which was dissolved in the mixture of 0.1 N 1-10 (20 mL) and ethyl acetate (40 mL). The organics were washed with H₂O and brine and dried over Na₂SO₄. The solution was evaporated and the residue was purified by silica gel column to give (6) (410 mg, 34%) as white foam, LCMS 409.1 [M+H]⁺.

### Step 1.7. Preparation of (7A) and (7B)

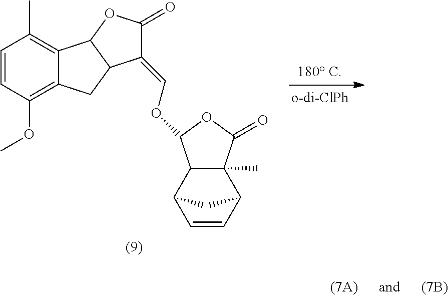

A mixture of cyclo-adduct (9) (400 mg, 0.98 mmol) in o-dichlorobenzene (50 mL) was heated at 180° C. for 25 h. After being cooled to rt, the solvent was removed in vacuo. The residue was purified by silica gel column to give (7A) (40 mg, 27%, fast moving spot on TLC) and (7B) (60 mg, 40%, slow moving spot on TLC) as white foam. Part of (10) was recovered (220 mg).

Compound (7A): LC-MS: 343.1 [M+H]^{+ 1}H NMR (300 MHz, CDCl₃) δ 7.49 (d, J=2.7 Hz, 1H), 7.04 (d, J=7.8 Hz, 1H), 6.97 (t, J=1.8 Hz, 1H), 6.73 (d, J=8.7 Hz, 1H), 6.16 (t, J=1.2 Hz, 1H), 5.99 (d, J=7.8 Hz, 1H), 3.93 (m, 1H), 3.80 (s, 3H), 3.35 (dd, J=9.6 Hz, 17.9 Hz, 1H), 2.99 (dd, J=3.6, 17.4 Hz, 1H), 2.38 (s, 3H), 2.04 (s, 3H).

Compound (7B): LC-MS: 343.1[M+H] ¹H NMR (300 MHz, CDCl₃) δ 7.48 (d, J=2.7 Hz, 1H), 7.04 (d, J=7.8 Hz, 1H), 6.95 (t, J=1.5 Hz, 1H), 6.73 (d, J=8.4 Hz, 1H), 6.17 (t, J=1.2 Hz, 1H), 5.99 (d, J=7.8 Hz, 1H), 3.94 (m, 1H), 3.80 (s, 3H), 3.34 (dd, J=9.6 Hz, 17.911z, 1H), 2.99 (dd, J.=3.6, 17.4 Hz, 1H), 2.37 (s, 3H), 2.05 (s, 3H).

### Example 2

The terpenoid lactones having the molecular structure of formula (1A) and (1B) were synthesized as described below.

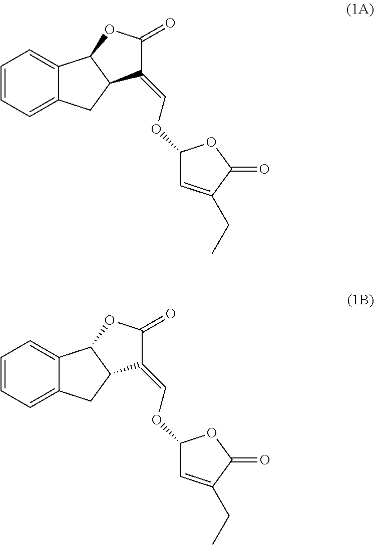 [SMILES: CCC1=C[C@H](O/C=C2/C(=O)O[C@@H]3c4ccccc4C[C@H]23)OC1=O ; CCC1=C[C@H](O/C=C2/C(=O)O[C@H]3c4ccccc4C[C@@H]23)OC1=O]

The synthetic procedures used in this example were identical to those described in Example 1 except that the starting material used was 3-ethyl-furan-2,5-dione instead of 3-methyl-furan-2,5-dione in Step 1.1.

Step 2.2: Reduction, analogous to Step 1.2 in Example 1 (69%). Step 2.3: Mentholoxy lactone preparation, analogous to Step 1.3 in Example 1 (23%). Step 2.4: TFA cleavage, analogous to Step 1.4 in Example 1 (86%). Step 2.5: Preparation of the ex-chloro lactone (10), analogous to Step 1.5 in Example 1.

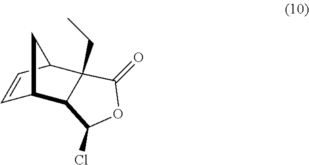

Chloro-lactone (12): ¹H NMR (300 MHz, CDCl₃) 6.30 (dd, J=3.0, 5.7 Hz, 1H), 6.22 (dd, J=3.0, 5.7 Hz, 1H), 5.64 (s, 1H), 3.25 (m, 1H), 3.03 (dd, J=1.2, 3.9 Hz, 1H), 2.93 (m, 1H), 2.14 (dd, J=4.8, 13.5 Hz, 1H), 1.72 (m, 1H), L67 (q, J=1.8 Hz, 2H), 1.13 (t, J=7.5 Hz, 3H).

### Step 2.6. Preparation of intermediate (11)

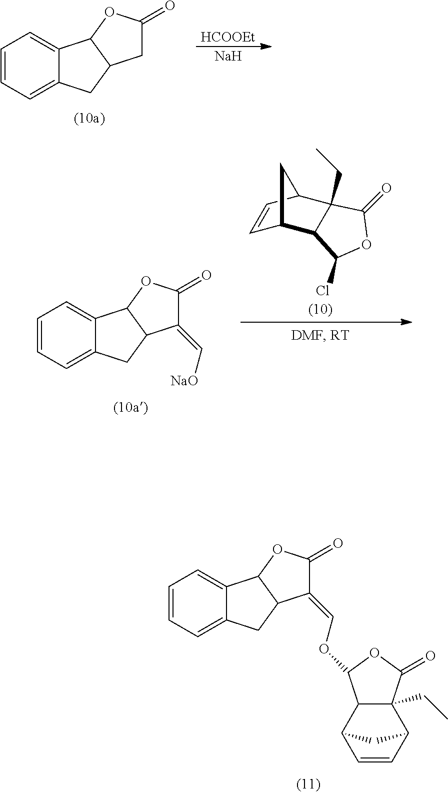

Step 2.6. Preparation of intermediate (11) followed the general procedure of Example 1, Step 1.6, using lactone (10a) and exo-chloro lactone (10) as starting materials. Yield: 88%. LC-MS: 379.1 [M+H]⁺.

Step 2.7. Preparation of compounds (1A) and (1B) followed the general procedure of Example 1, Step 1.7, using cyclo-adduct 13. Compound (1A): Fast moving spot on TLC. Yield: 26%. LCMS 313.1 [M+H]⁺. ¹H NMR (300 MHz, CDCl₃) δ 7.51 (d, J=6.9 Hz, 1H), 7.49 (d, J=2.4 Hz, 1H), 7.36-7.22 (m, 3H), 6.92 (d, J=1.5 Hz, 1H), 6.18 (d, J=1.2 Hz, 1H), 5.96 (d, J=7.8 Hz, 1H), 3.95 (m, 1H), 3.45 (dd, J=9.3, 17.1 Hz, 1H), 3.11 (dd, J=3.3, 17.1 Hz, 1H), 2.42 (q, J=7.5 Hz, 21-1), 1.24 (t, J 7.5 Hz, 3H). Compound (1B): Slow moving spot on TLC.

Yield: 44%. LCMS 313.1 [M+H]⁺. ¹HNMR (300 MHz, CDCl₃) δ 7.50 (d, J=7.2 Hz, 1H), 7.48 (d, J-2.4 Hz, 1H), 7.36-7.22 (m, 3H), 6.92 (d, J=1.5 Hz, 1H), 6.18 (d, J=1.2 Hz, 1H), 5.96 (d, J=7.8 Hz, 1H), 3.94 (m, 1H), 3.42 (dd, J=6.9, 16.8 Hz, 1H), 3.10 (dd, J=3.3, 16.8 Hz, 1H), 2.42 (q, J=7.5 Hz, 2H), 1.24 (t, J=7.5 Hz, 3H).

### Example 3

The terpenoid lactones having the molecular structure of formula (5A) and (5B) were synthesized as follows.

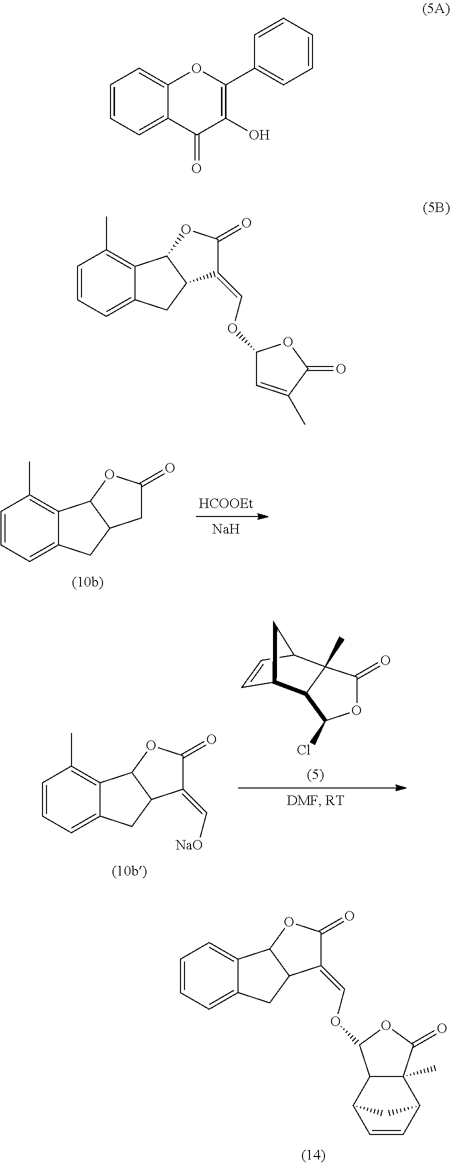

Step 3.1. To a solution of tricyclic lactone (10b) (470 mg, 2.5 mmol) in ethyl formate (20 mL) at 0° C. was added, under nitrogen, 1.2 eq NaH (112 mg, 2.8 mmol). The mixture was allowed to warm to room temperature and stirred for 3 h. When TLC analysis indicated complete formylation, excess ethyl formate was removed under reduced pressure. The sodium salt (10b′) was dissolved in DMF (20 mL) and cooled to 0° C. Upon addition of exo-5-(S)-chlorolactone (5) (397 mg, 2 mmol), the mixture was stirred overnight. DMF was removed in vacuo. The residue was dissolved in the mixture of 0.1 N HCl (20 mL) and ethyl acetate (40 mL). And the organics were washed with H₂O and brine and dried over Na₂SO₄. The solution was evaporated and the residue was purified by silica gel column to give (14) (650 mg, 88%) as white foam. LC-MS: 379.1 [M+H]⁺.

Step 3.2. Preparation of compounds (5a) and (5b) from (14): Cycloadduct (14) (300 mg, 0.815 mmol) in o-dichlorobenzene (50 mL) was heated at 180° C. for 15 h. The solvent was removed in vacuo to give a residue, which was purified by silica gel to give (5A) (40 mg, 24%, fast moving spot on TLC) and (5B) (60 mg, 37%, slow moving spot on TLC) as white foam, and recovered (14) (100 mg). Compound (5A): LC-MS: 313.1 [M+H]⁺. ¹HNMR (300 MHz, CDCl₃) δ 7.49 (d, J=2.7 Hz, 1H), 7.24 (m, 1H0, 7.06 (m, 2H), 6.97 (m, 1H), 6.17 (m, 1H), 6.00 (d, J=7.8 Hz, 1H), 3.93 (m, 1H), 3.43 (dd, J 9.3, 16.8 Hz, 1H), 3.08 (dd, J=3.9, 17.4 Hz, 1H), 2.44 (s, 3H), 2.03 (s, 3H). Compound (5B): LC-MS: 313.1 [M+H]⁺. ¹HNMR (300 MHz, CDCl₃) δ 7.48 (d, J=2.7 Hz, 1H), 7.24 (m, 1H0, 7.06 (m, 2H), 6.97 (m, 1H), 6.18 (m, 1H), 6.00 (d, J=7.8 Hz, 1H), 3.92 (m, 1H), 3.42 (dd, J=9.3, 16.8 Hz, 1H), 3.07 (dd, J=3.9, 17.4 Hz, 1H), 2.44 (s, 3H), 2.04 (s, 3H).

### Example 4

The terpenoid lactones having the molecular structure of formula (6A) and (6B) were synthesized as follows

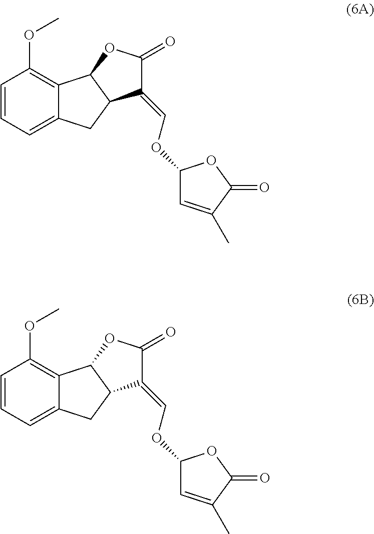 [SMILES: COc1cccc2c1[C@H]1OC(=O)/C(=C/O[C@H]3C=C(C)C(=O)O3)[C@H]1C2 ; COc1cccc2c1[C@@H]1OC(=O)/C(=C/O[C@H]3C=C(C)C(=O)O3)[C@@H]1C2]

Step 4.1. Preparation of intermediate (15) followed the general procedure of Example 1, Step 1.6, using lactone (10e) and chloride (5) as starting materials. Yield: 79%: LC-MS: 395.1 [M+H]⁺

Step 4.2. Preparation of compounds (6A) and (6B) followed the general procedure of Example 1, Step 1.7, using cyclo-adduct (15).

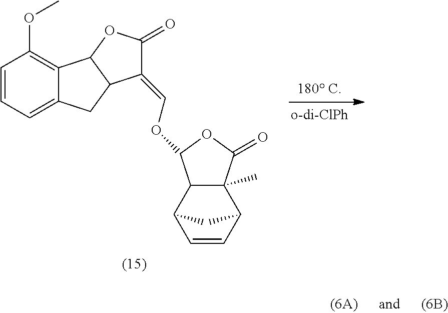

Compound (6A): Yield: 24%. LC-MS: 329.1 [M+H]⁺. ¹HNMR (300 MHz, CDCl₃) δ 7.47 (d, J=2.4 Hz, 1H), 7.31 (t, J=7.8 Hz, 1H), 6.95 (m, 1H), 6.80 (d, J 7.8 Hz, 1H), 6.73 (d, J=8.1 Hz, 1H), 6.17 (m, 1H), 6.05 (d, J=8.1 Hz, 1H), 3.93 (m, 1H), 3.88 (s, 3H), 3.41 (m, 1H), 3.04 (dd, J=3.9, 16.8 Hz, 1H), 2.04 (s, 3H). Compound (6B): Yield: 30%. LC-MS: 329.1 [M+H]⁺. ¹HNMR (300 MHz, CDCl₃) δ 7.47 (d, J=2.4 Hz, 1H), 7.31 (t, J=7.8 Hz, 1H), 6.95 (m, 1H), 6.80 (d, J=7.8 Hz, 1H), 6.73 (d, J=8.1 Hz, 1H), 6.17 (m, 1H), 6.05 (d, J=8.1 Hz, 1H), 3.92 (m, 1H), 3.88 (s, 3H), 3.42 (dd, 9.6, 17.1 Hz, 1H), 3.04 (dd, J=3.9, 16.8 Hz, 1H), 2.04 (s, 3H).

### Example 5

The terpenoid lactones having the molecular structure of formula (8A) and (8B) were synthesized as follows.

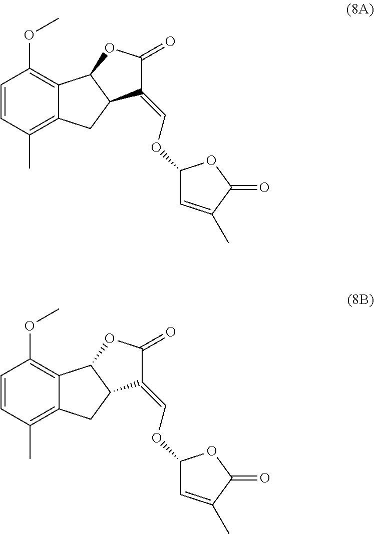 [SMILES: COc1ccc(C)c2c1[C@H]1OC(=O)/C(=C/O[C@H]3C=C(C)C(=O)O3)[C@H]1C2 ; COc1ccc(C)c2c1[C@@H]1OC(=O)/C(=C/O[C@H]3C=C(C)C(=O)O3)[C@@H]1C2]

Step 5.1. Preparation of intermediate (16) followed the general procedure of Example 1, Step L6, using lactone (10e) and chloride (5) as starting materials. Yield: 21%; LC-MS: 409.1 [M+H]⁺

Step 5.2. Preparation of compounds (8A) and (8B) followed the general procedure of Example 1, Step 1.7, using cyclo-adduct

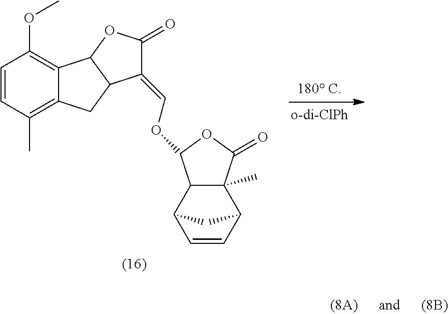

Compound (8A): Yield: 12%. LC-MS: 343.1 [M+H]⁺. ¹HNMR (300 MHz, CDCl₃) δ 7.48 (d, J=2.4 Hz, 1H), 7.10 (d, J=8.1 Hz, 1H), 6.97 (m, 1H), 6.66 (d, J=8.1 Hz, 1H), 6.18 (t, J=1.2 Hz, 1H), 6.04 (d, J=8.1 Hz, 1H), 3.93 (m, 1H), 3.84 (s, 3H), 3.32 (dd, J=8.4 Hz, 17.1 Hz, 1H), 2.89 (dd, J=3.9, 17.1 Hz, 1H), 2.15 (s, 3H), 2.04 (s, 3H). Compound (8B): Yield: 30%.

LC-MS: 343.1 [M+H]⁺. ¹HNMR (300 MHz, CDCl₃) δ 7.48 (d, J=2.4 Hz, 1H), 7.11 (d, J=8.1 Hz, 1H), 6.98 (m, 1H), 6.66 (d, J=8.1 Hz, 1H), 6.20 (t, J=1.2 Hz, 1H), 6.05 (d, J=7.8 Hz, 1H), 3.93 (m, 1H), 3.85 (s, 3H), 3.32 (dd, J=8.4 Hz, 17.1 Hz, 1H), 2.90 (dd, J=3.9, 17.1 Hz, 1H), 2.17 (s, 3H), 2.04 (s, 3H).

### Example 6

The terpenoid lactones having the molecular structure of formula (4A) and (4B) were synthesized as follows.

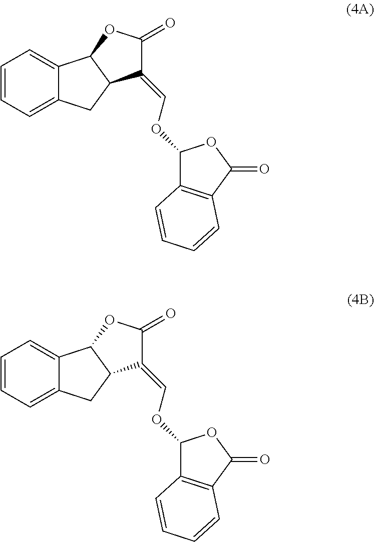 [SMILES: O=C1O[C@@H]2c3ccccc3C[C@@H]2/C1=C\O[C@@H]1OC(=O)c2ccccc21 ; O=C1O[C@H]2c3ccccc3C[C@H]2/C1=C\O[C@@H]1OC(=O)c2ccccc21]

Step 6.1. Preparation of intermediate (16) followed the general procedure of Example 1, Step 1.6, using lactone (10a) and bromide (17) as starting materials.

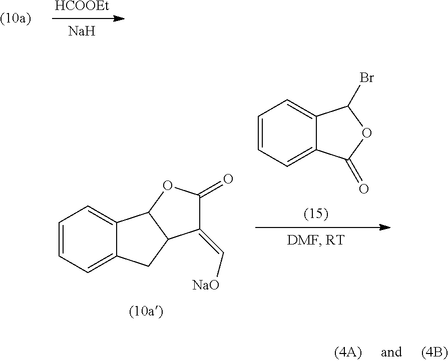

Compound (4A): Yield: 28%. LC-MS: 335 [M+H]⁺. ¹HNMR (300 MHz, CDCl₃) δ 8.00 (d, J=7.5 Hz, 1H), 7.87-7.67 (m, 3H), 7.55 (d, J=2.7 Hz, 1H), 7.51 (d, J=6.6 Hz, 1H), 7.35-7.23 (m, 3H), 6.72 (s, 1H), 5.96 (d. J=7.8 Hz, 1H), 3.94 (m, 1H), 3.41 (dd, J=9.6, 17.1 Hz, 1H), 3.13 (dd, J=3.3, 16.8 Hz, 1H). Compound (403): Yield 28%. LC-MS: 357 [M+Na]⁺. ¹HNMR (300 MHz, CDCl₃) δ 8.00 (d, J=7.5 Hz, 1H), 7.85-7.66 (m, 3H), 7.54 (d, J=2.7 Hz, 1H), 7.49 (d, J=6.6 Hz, 1H), 7.33-7.20 (m, 3H), 6.72 (s, 1H), 5.96 (d, J=7.8 Hz, 1H), 3.96 (m, 1H), 338 (dd, J=9.6, 17.1 Hz, 1H), 3.09 (dd, J=33, 16.8 Hz, 1H).

### Example 7

The terpenoid lactones having the molecular structure of formula (3A) and (313) were synthesized as follows,

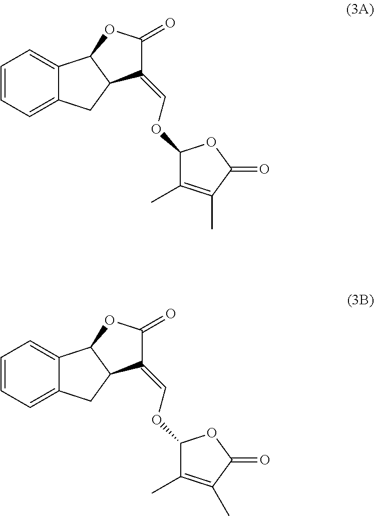 [SMILES: CC1=C(C)[C@@H](O/C=C2/C(=O)O[C@@H]3c4ccccc4C[C@H]23)OC1=O ; CC1=C(C)[C@H](O/C=C2/C(=O)O[C@@H]3c4ccccc4C[C@H]23)OC1=O]

### Step 7.1. Preparation of chloride (18)

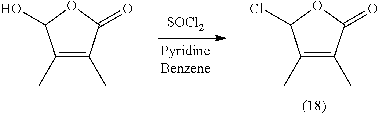

To a solution of 5-hydroxy-3,4-dimethyl-5H-furan-2-one (600 mg, 5.26 mmol) in benzene (8 mL) was added pyridine (0.85 mL, 10.5 mmol). Thionyl chloride (0.76 mL, 10.5 mmol) was added dropwise to the solution. The mixture was stirred at rt for 10 min. The solvent was removed in vacuo. The crude mixture was passed a short silica gel column (AcOEt: Hexanes, 1:1) to afford chloride (18) (600 mg, 78%). ¹H NMR (300 MHz, CDCl₃) δ 6.38 (s, 1H), 2.08 (s, 3H), 1.90 (s, 3H).

Step 7.2. To a solution of tricyclic lactone (10a) (300 mg, 132 mmol) in ethyl formate (15 mL) at 0° C. was added NaH (83 mg, 2.07 mmol). The mixture was allowed to warm to room temperature and stirred for 5 hrs. The solvent was removed in vacuo. The crude product (10a′) (see Example 6) was used directly in the next step. To the sodium salt of formylated) (10a° (crude, 1.72 mmol) in THF (10 mL) was added chloride (18) (273 mg, 2.07 mmol) in THF (5 mL) at 0° C. The reaction mixture was stirred at room temperature over weekend. The solvent was removed in vacuo. The residue was dissolved in a mixture of brine and ethyl acetate. The aqueous phase was extracted with ethyl acetate. The combined organic layer was washed with saturated NH₄Cl, dried and concentrated. The crude mixture was purified by flash column chromatography (AcOEt: hexanes, 1:2) to afford (3A) (75 mg, fast moving spot on TLC) and (3B) (75 mg, slow moving spot on TLC).

Compound (3A): LC-MS: 313 (M+H)⁺. ¹H NMR (300 MHz, CDCl₃) δ 7.51 (d, 1H), 7.47 (d, 1H), 7.35-7.19 (m, 3H), 5.96 (m, 2H), 3.96 (m, 1H), 3.46 (dd, 1H), 3.12 (dd, 1H), 2.06 (s, 3H), 1.92 (s, 3H). Compound (3B): LC-MS: 313 (M+H)⁺. ¹H NMR (300 MHz, CDCl₃) δ 7.50 (d, 1H), 7.44 (d, 1H), 7.35-7.19 (m, 3H), 5.97 (m, 2H), 3.95 (m, 1H), 3.45 (dd, 1H), 3.10 (dd, 1H), 2.05 (s, 3H), 1.92 (s, 3H).

### BIOLOGICAL EVALUATION

### Procedures Used in Examples 8-14

Cell Culture:

3T3L1 cells were purchased from American Type Culture Collection (Manassas, Va., USA) and cultured in DMEM media containing 4.5 g/l glucose supplemented with 10% fetal bovine serum (Hyclone), 2 mM L-glutamine (Gibco), 100 U/ml penicillin, and 100 μg/ml streptomycin (Gibco). The cells were cultured at 37° C. in a humidified atmosphere with 10% CO₂.

The 3T3L1 cells were plated at 6×10⁴ cells/well in 12 well plates and incubated at 37° C. in a humidified atmosphere with 10% CO₂. The media was removed after 24 hours and the cells were treated with 60 μM GR24 individually and in combination with 60 resveratrol and 60 μM pinosylvin, and incubated for 24 hours at 37° C. in a humidified atmosphere with 10% CO₂. The media was removed, cells were washed with PBS, and cell lysates were collected on ice. Proteins were extracted using RIPA buffer along with protease and phosphatase inhibitors. Lysates were centrifuged for 30 min at high speed; supernatant was collected and stored at 70° C. until further analysis. The vehicle controls were 3T3 L1 cells without any treatment with drugs or other compounds. The drugs or compounds that were used in all the experiments were dissolved or diluted in dimethylsulfoxide (DMSO) as diluent. In all experiments, the controls were treated with the same volume of DMSO as was used to dissolve drugs or compounds in compound-treated cells to show and ensure that the actual change in protein expression in different treatments was due solely to the compounds or combination of compounds and not to the DMSO.

Western Blotting:

Protein concentrations were measured using a BCA (bicinchoninic acid) protein assay kit (Cat.#23225; Pierce, Rockford, Ill.). Twenty (20) μg/lane of total cellular protein samples containing NuPAGE LDS sample buffer (Invitrogen) and reducing agent were loaded into 4-12% NuPAGE Bis-Tris gels (Invitrogen), subjected to gel electrophoresis, and transferred to polyvinylidene fluoride membranes (Amersham). For SIRT1 proteins, membranes were blocked in 0.05% TBS-Tween with 3% milk for 1 hour, incubated overnight at +4° C. with SIRT1 primary antibodies (Cat.#07-131, Millipore). For phospho-AMPKα and AMPKα proteins, blocking was done for 1 hour in 0.1% TBS-Tween with 5% milk. The membranes were incubated overnight at +4° C. with phospho-AMPKα (Cat. #2535, Cell Signaling) and AMPKα (Cat.#2532, Cell Signaling) primary antibodies. Horseradish peroxidase-conjugated anti-rabbit antibodies (Cat. #NA934V GE Health Care, Amersham, U.K) were used as secondary antibodies. For α-tubulin (loading control), membranes were blocked in 0.05% PBS-Tween with 3% milk for 1 hour, incubated with α-tubulin (Cat.# B-5-1-2, Sigma) primary antibodies for 1 hour at room temperature and horseradish peroxidase-conjugated anti-mouse (Cat. # NA 931V GE Health Care, Amersham, U.K.) IgG antibodies were used as secondary antibodies.

The membranes were developed using chemiluminescence (ECL plus, GE Health Care), and images were captured in an Image Quant RT-ECL machine (version 1.0.1; GE Health Care). Densitometry and quantification of the bands were done by applying Quantity One software (Bio-Rad). The expression levels of the proteins were normalized to α-tubulin protein levels.

Mitochondrial Staining.

The 3T3L1 preadipocytes were plated onto Ibidi u-slide 8 well plates at 1.6×10⁴ cells/well and incubated at 37° C. in a humidified atmosphere with 10% CO₂. The media was removed after 24 hours, the cells were treated with 60 μM GR24 and incubated for 24 hours at 37° C. in a humidified atmosphere with 10% CO₂. After 24 hours of treatment, staining of mitochondria was done using 200 nM MitoTracker Mitochondrion-Selective Probes (Green FM probes Cat.#M7514, Invitrogen) according to the manufacturer's instructions. The cells were observed using a confocal microscope and images were taken at 40× oil immersion.

### Example 8

Treatment of 3T3L1 preadipocytes with synthetic strigolactone analog GR24:

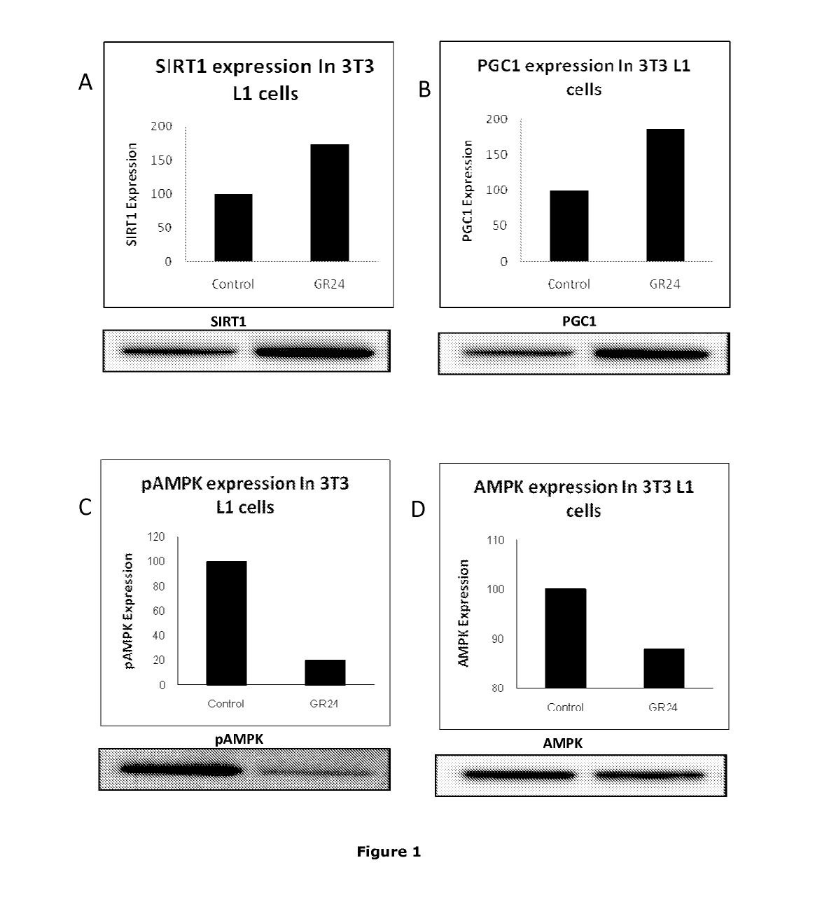

3T3L1 preadipocytes were treated with 100 μM GR24 for 24 hours. The immunoblots were quantitated by Quantity One software, the relative protein concentrations were normalized to α-tubulin and the graphs were plotted. The effects of a synthetic analog of strigolactone G24 on energy metabolism in tissue cultures were revealed. GR24 treatment of adipocytes significantly increased SIRT1 protein expression (FIG. 1A) as well as PPAR-gamma coactivator 1 (PGC-1α, a master regulator of mitochondrial biogenesis) expression (FIG. 1B), which is responsible for mitochondrial biogenesis. In contrast, phosphorylated (active form) AMPK (FIG. 1C), total AMPK (FIG. 1D), phosphorylated ACC (FIG. 1E) and a target of AMPK activation, the protein expression of acetyl-CoA carboxylase (ACC), a downstream target of AMPK (FIG. 1F), were down-regulated.

PGC-1α as an indicator of SIRT1 activity: PGC-1α has been extensively described as a master regulator of mitochondrial biogenesis. The metabolic sensor SIRT1 has been shown to directly affect PGC-1α protein expression and activity through phosphorylation and deacetylation, respectively. Recent insights suggest that SIRT1 and PGC-1α might act as an orchestrated network to improve metabolic fitness (Canto C et al., Curr Opin Lipidol 2009). When SIRT1 protein expression is induced, it interacts with and deacetylates PGC-1α in an NAD-dependent manner. Thus SIRT1 acts as a modulator of PGC-1a (Rodgers J T et al, Nature 2005). The effects of resveratrol, a nonselective SIRT1 activator, were associated with an induction of genes for oxidative phosphorylation and mitochondrial biogenesis, and were largely explained by a resveratrol-mediated decrease in PGC-1α acetylation and an increase in PGC-1α activity (Lagouge M, Cell 2006). Therefore, PGC-1α protein expression serves as an indicator of SIRT1 activity.

### Example 9

The effect of GR24 and resveratrol on SIRT1 expression:

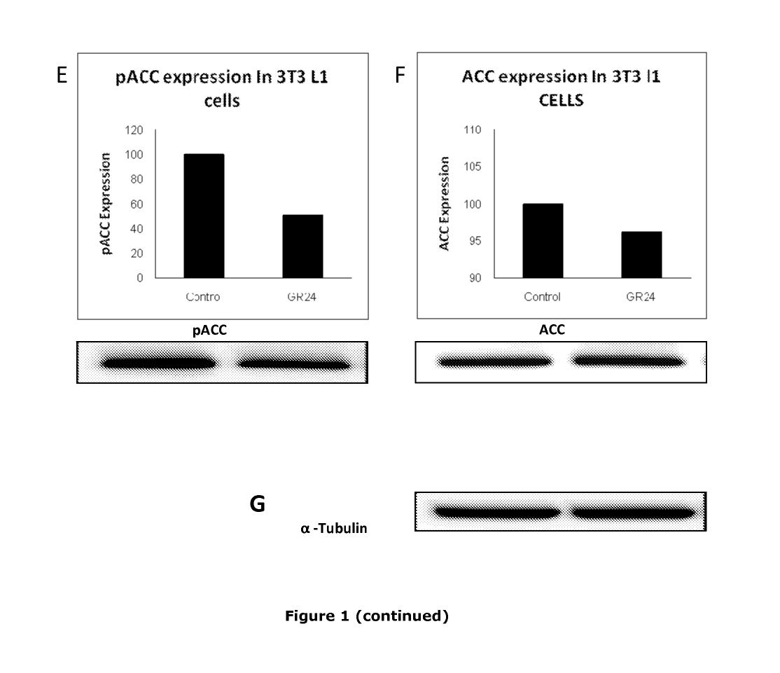

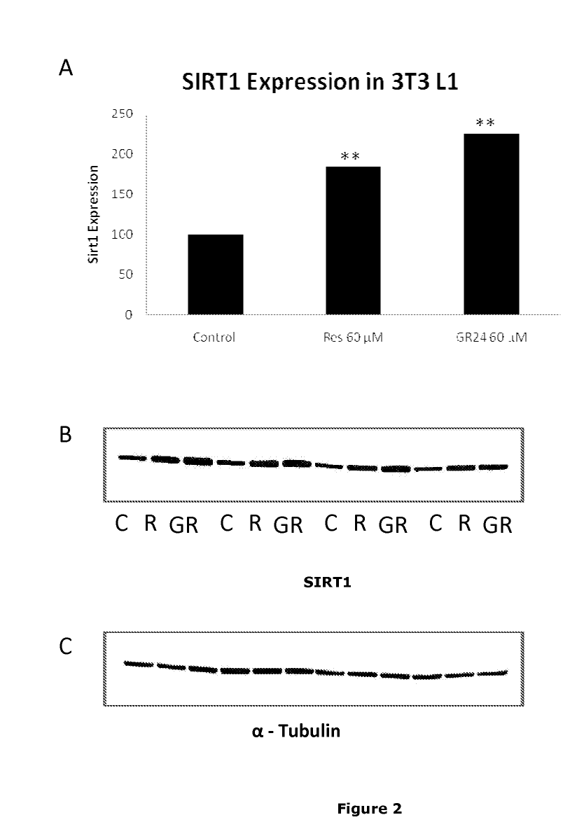

3T3 L1 preadipocytes were treated with 60 μM resveratrol and 60 μM GR24 for 24 hours. Quantitation of immunoblots was done by Quantity One software, SIRT1 protein concentration was normalized to α-tubulin, and the data are expressed as percentages of control (mean±SEM) from four independent experiments. Statistical significance was assessed by Student's t-test:**P<0.01. A significant increase of SIRT1 protein expression in cells treated with GR24 compared to control was observed (FIG. 2A). A dose of 60 μM GR24 increased SIRT1 expression greater than did resveratrol. FIG. 2B shows the immunoblots of SIRT1 and α-tubulin.

### Example 10

The effect of GR24 and resveratrol on pAMPK and AMPK expression:

3T3 L1 preadipocytes were treated with 60 μM resveratrol and 60 μM GR24 for 24 hours. Quantitation of immunoblots was done by applying Quantity One software, individual protein concentrations were normalized to α-tubulin and the data are expressed as percentages of control (mean±SEM) from four independent experiments. Statistical significance was assessed by Student's t-test:*P<0.05.

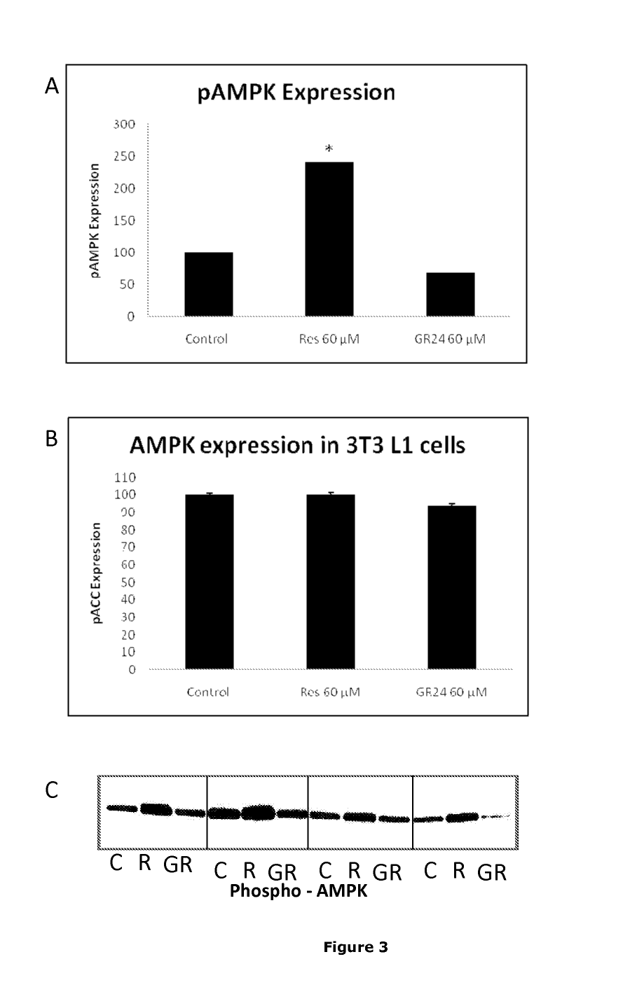

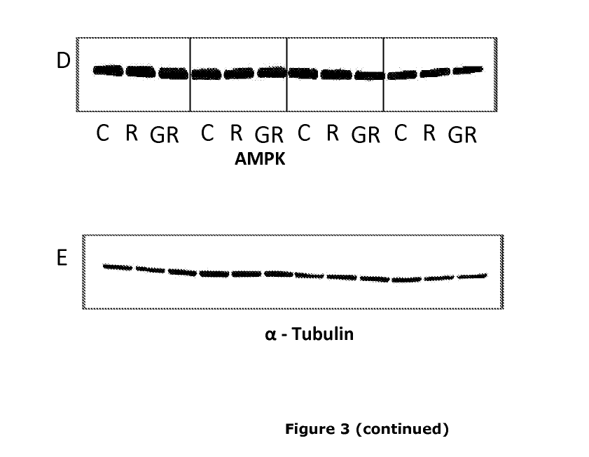

FIG. 3A shows densitometry of phospho-AMPK, which shows a significant increase in expression with resveratrol but not with GR24. FIG. 3B shows AMPK expression in the same blot obtained after stripping and reprobing. FIG. 3C represents the western blot images of phospho-AMPK, AMPK and α-tubulin.

The results presented in FIG. 3 indicate that a dose of 60 μM resveratrol increased pAMPK expression whereas GR24 did not, implying that the inhibitory effect of GR24 on pAMPK expression is dose-dependent and happens only with a high dose of GR24.

### Example 11

The effect of GR24 and resveratrol on pACC and ACC expression:

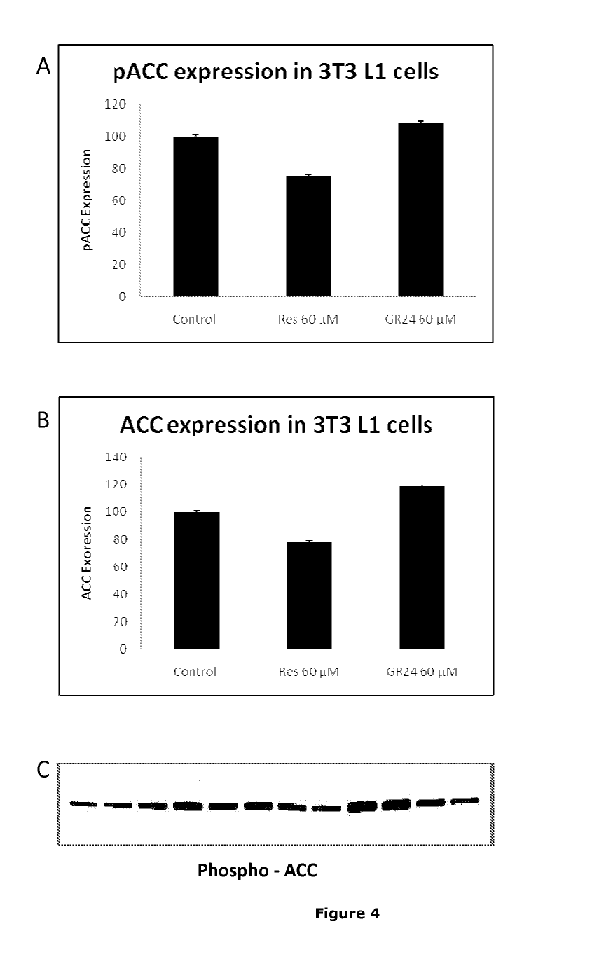

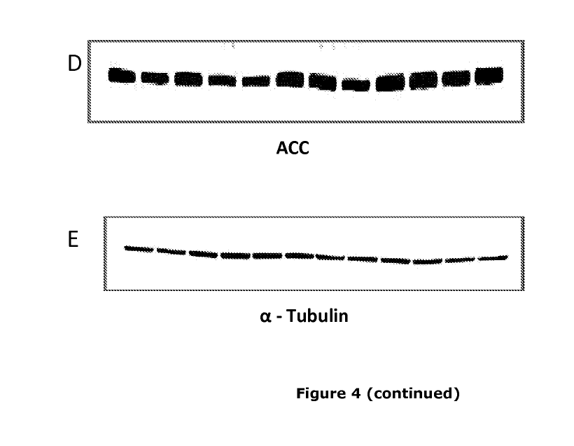

3T3 L1 preadipocytes were treated with 60 μM resveratrol and 60 μM GR24 for 24 hours. Quantitation of immunoblots was done by applying Quantity One software, relative protein concentrations were normalized to α-tubulin and the data are expressed as percentages of control (mean±SEM) from four independent experiments. Statistical significance was assessed by Student's t-test. FIG. 4 shows the results of immunoblots and densitometry. It is shown that with a dose of 60 μM resveratrol or GR24 there is no change in phosphorylation of ACC implying again that the inhibitory effect of GR24 on phosphorylation of ACC is dose-dependent and happens only at higher doses of GR24. There was no change in phospho-ACC expression compared to the control (FIG. 4A). FIG. 4B shows the expression level of ACC. FIG. 4C represents the immunoblots of phospho-ACC, ACC and α-tubulin.

### Example 12

The effect of GR24 and resveratrol on mitochondria shape and density:

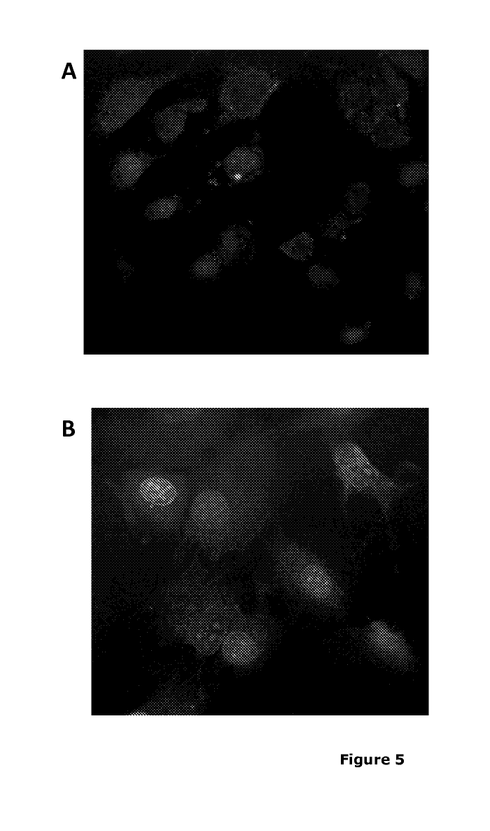

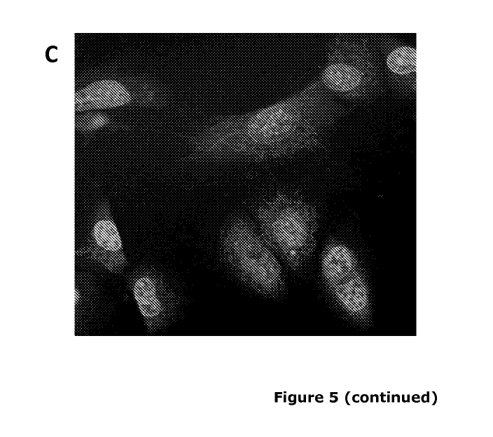

Preadipocytes were treated with DMSO (Vehicle control) (FIG. 5A), 60 μM resveratrol (FIG. 5B) or 60 μM strigolactone analog GR24 (FIG. 5C) for 24 hours. Next, preadipocytes were stained with MitoTracker green and observed under a confocal microscope at 40× oil immersion. FIG. 5 shows mitochondrial staining with fluorescent MitoTracker green, which binds specifically to mitochondria. The white oval shaped structures are nucleus and the white thread-like structures around the nucleus in the cytoplasm are mitochondria. When compared to the control, there was an increase in mitochondrial biogenesis represented by elongated mitochondria, and an increase in mitochondrial activation represented by the intensity of fluorescence, in cells treated with resveratrol and GR24. The increase in intensity of staining is clearly visible only in the GR24 treated cells. Thus, GR24 enhances both biogenesis and activity of mitochondria, which is necessary to generate ATP.

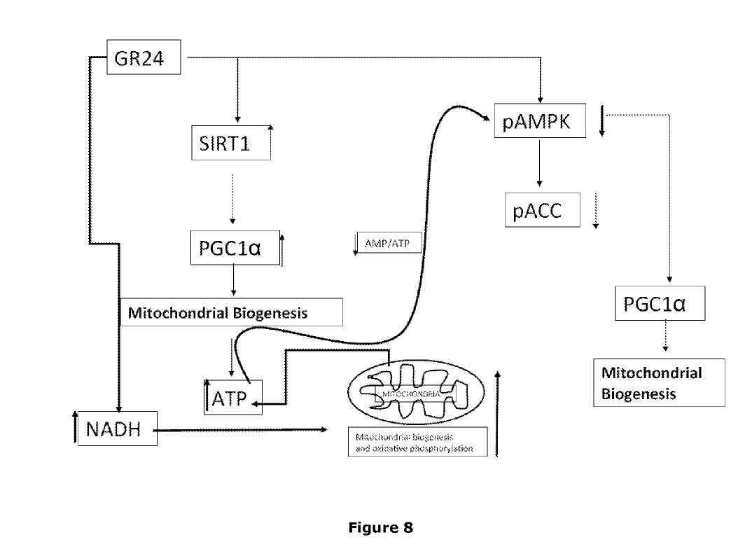

In summary, it was shown that GR24 is a specific activator of NADH and SIRT1, and does not activate the AMPK system. The effects of GR24 on energy regulation are shown in FIG. 8.

### Example 13

The effects of synthetic strigolactone analog GR24 alone or in combination with resveratrol and/or pinosylvin on SIRT1 expression in 3T3 L1 cells:

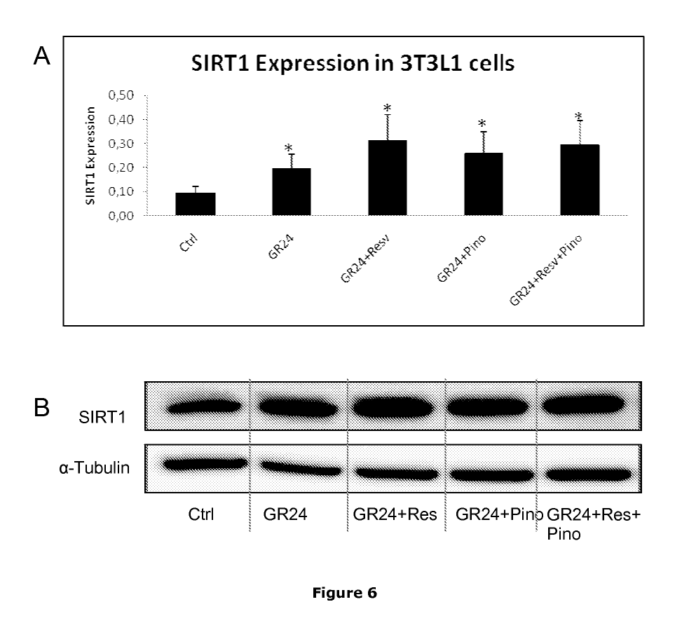

3T3 L1 cells were treated with 60 μM GR24 alone or in combination as follows: GR24 and resveratrol; GR24 and pinosylvin; GR24 and resveratrol and pinosylvin. Densitometry of immunoblots was done by applying Quantity One software, SIRT1 protein concentration was normalized to α-tubulin; the data are represented as means±SEM from four independent experiments and were analyzed using the Wilcoxon test. SIRT1 protein expression was significantly (*P<0.05) increased with all the treatments compared to the control (FIG. 6A). Significant increase in SIRT1 (*P<0.05) was also observed when GR24 treated cells were compared with GR24 and resveratrol treatment. FIG. 6B depicts corresponding Western blotting results of SIRT1 and tubulin (used as loading control).

Treatment of 3T3 L1 cells with GR24 alone and combined treatments with resveratrol and/or pinosylvin significantly increased SIRT1 expression compared to control. The combined treatment with GR24 and resveratrol augmented the expression of SIRT1 significantly more than the treatment with GR24 alone (P=0.012).

### Example 14

The effects of synthetic strigolactone analog GR24 alone or in combination with resveratrol and/or pinosylvin on AMPK expression in 3T3L1 cells:

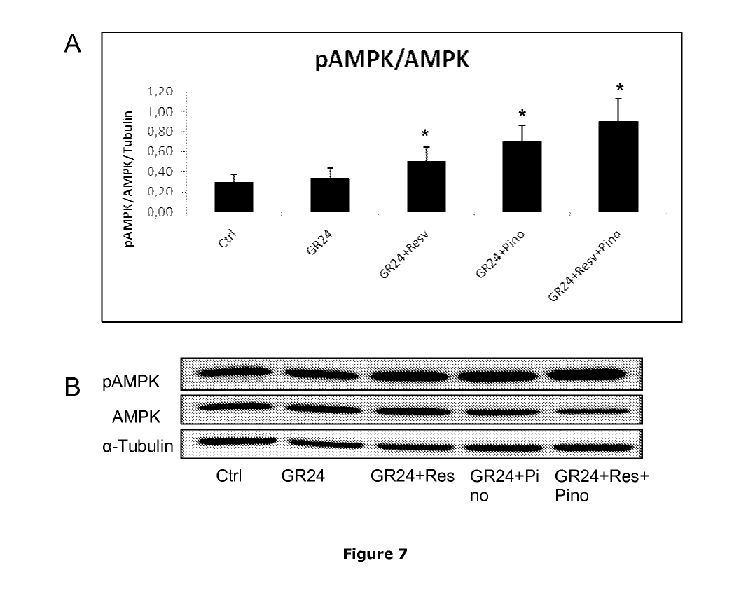

3T3 L1 preadipocytes were treated with 60 μM GR24 alone or in combination as follows: GR24 and resveratrol; GR24 and pinosylvin; GR24 and resveratrol and pinosylvin, for 24 hours. Western blots and densitometry showing AMPK-activation expression levels are presented in FIG. 7. Quantitation of immunoblots was done by applying Quantity One software, and relative protein concentrations were normalized to α-tubulin. The data are expressed as means±SEM from four independent experiments and were analyzed using the Wilcoxon test. FIG. 7A depicts AMPK activation (pAMPK/AMPK/α-tubulin ratio) in cultured 3T3 L1 cells treated with 60 μM GR24 alone or in combination as follows: GR24 and resveratrol; GR24 and pinosylvin; GR24 and resveratrol and pinosylvin. FIG. 7B depicts corresponding Western blotting results of pAMPK, AMPK and α-tubulin (used as loading control).

There was no activation of AMPK (expressed as pAMPK/AMPK) in cultured cells treated with GR24 alone (FIG. 7B). However, the combined treatment with GR24 and resveratrol or pinosylvin or with both resveratrol and pinosylvin augmented the activation of AMPK significantly, compared to control, in an increasing manner. This proves that the activation of AMPK is due to resveratrol and pinosylvin but not GR24, and therefore GR24 is a specific activator of SIRT1 and NADH and does not activate the AMPK system. The data indicates that GR24 alone did not activate AMPK but that the combined treatment of GR24 with resveratrol and/or pinosylvin significantly activated AMPK (*P<0.05). The above data suggest that GR24 acts though a different pathway than resveratrol and/or pinosylvin. Therefore, combined treatment with strigolactone or its derivatives and pinosylvin and/or resveratrol is beneficial for human metabolism at different conditions and can be used as dietary supplement or for treating or preventing metabolic disorders.

### Example 15

Biological evaluation of terpenoid lactones GR24, 1A, 1B, 5A, 5B, 6A, and 7A:

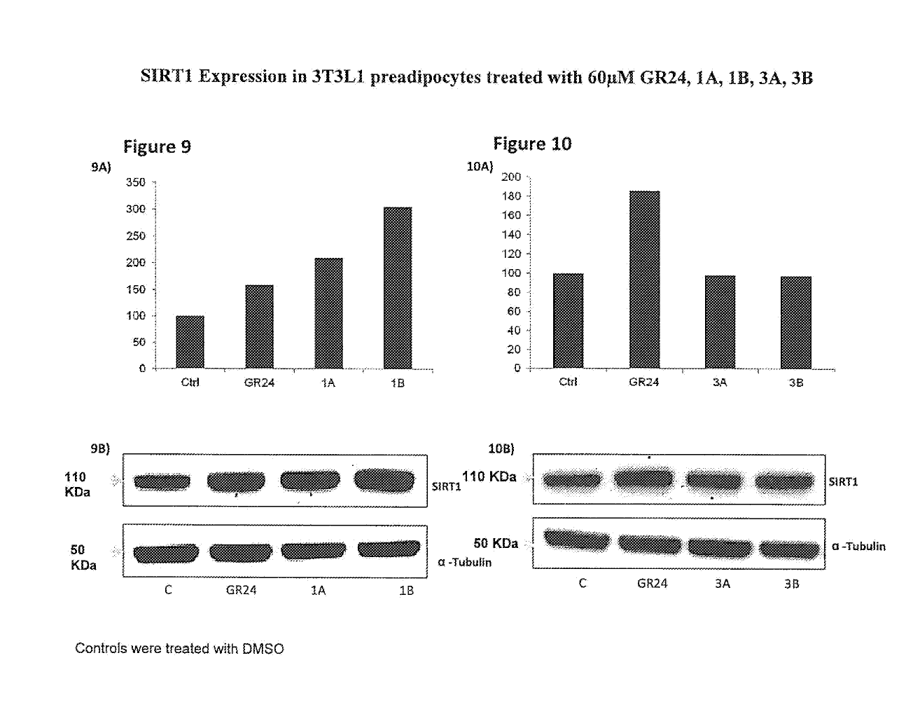

3T3L1 cells were purchased from American Type Culture Collection (Manassas, Va., USA) and cultured in DMEM media containing 4.5 g/l glucose supplemented with 10% fetal bovine serum (Hyclone), 2 mM L-glutamine (Gibco) and 100 U/ml penicillin, 100 μg/ml streptomycin (Gibco). The cells were cultured at 37° C. in a humidified atmosphere with 10% CO₂. 3T3L1 preadipocytes were plated at 1×10⁵ cells/well in 6 well plates and incubated at 37° C. in a humidified atmosphere with 10% CO₂. The media was removed after 24 hours and the cells were treated with 60 μM GR24 or with 60 μM 1A, 1B, 5A, 5B, 6A, or 7A and incubated for 24 hours at 37° C. in a humidified atmosphere with 10% CO₂. In all experiments, the controls were treated with the same volume of DMSO as was used to dissolve compounds in compound-treated cells to show and ensure that the actual change in protein expression in different treatments was only due to the compounds DMSO. The media was removed, cells were washed with PBS and cell lysates were collected on ice. Proteins were extracted using RIPA buffer along with protease and phosphatase inhibitors. Lysates were centrifuged for 30 min at high speed; supernatant was collected and stored at −70° C. until further analysis. The results are shown in FIGS. 9 through 37.

FIG. 9: SIRT1 Immunoblot and densitometry from 3T3 L1 preadipocytes treated with 60 μM GR24 or 60 μM 1A or 60 μM 1B for 24 hours. Bars show means of SIRT1 protein expression from one experiment with 2 replicates (FIG. 9A); no statistical analysis was performed. FIG. 9B shows the immunoblots of SIRT1 and α-tubulin. Quantitation of immunoblots was done by Quantity One software, and SIRT1 protein concentration was normalized to α-tubulin (used as a loading control).

FIG. 10: SIRT1 Immunoblot and densitometry from 3T3 L1 preadipocytes treated with 60 μM GR24 or 60 μM 3A or 60 μM 3B for 24 hours. Bars show means of SIRT1 protein expression from one experiment with 2 replicates (FIG. 10A); no statistical analysis was performed. FIG. 10B shows the immunoblots of SIRT1 and α-tubulin. Quantitation of immunoblots was done by Quantity One software, and SIRT1 protein concentration was normalized to α-tubulin (used as a loading control).

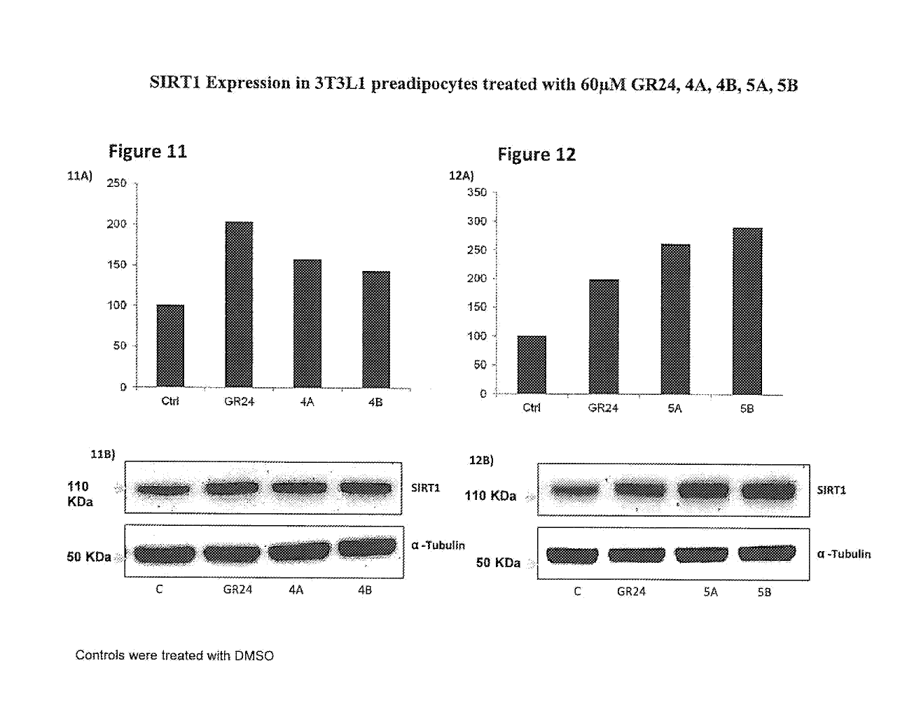

FIG. 11: SIRT1 Immunoblot and densitometry from 3T3 L1 preadipocytes treated with 60 μM GR24 or 60 μM 4A or 60 μM 4B for 24 hours. Bars show means of SIRT1 protein expression from one experiment with 2 replicates (FIG. 11A); no statistical analysis was performed. FIG. 11B shows the immunoblots of SIRT1 and α-tubulin. Quantitation of immunoblots was done by Quantity One software, and SIRT1 protein concentration was normalized to α-tubulin (used as a loading control).

FIG. 12: SIRT1 Immunoblot and densitometry from 3T3 L1 preadipocytes treated with 60 μM GR24 or 60 μM 5A or 60 μM 5B for 24 hours. Bars show means of SIRT1 protein expression from one experiment with 2 replicates (FIG. 12A); no statistical analysis was performed. FIG. 12B shows the immunoblots of SIRT1 and α-tubulin. Quantitation of immunoblots was done by Quantity One software, and SIRT1 protein concentration was normalized to α-tubulin (used as a loading control).

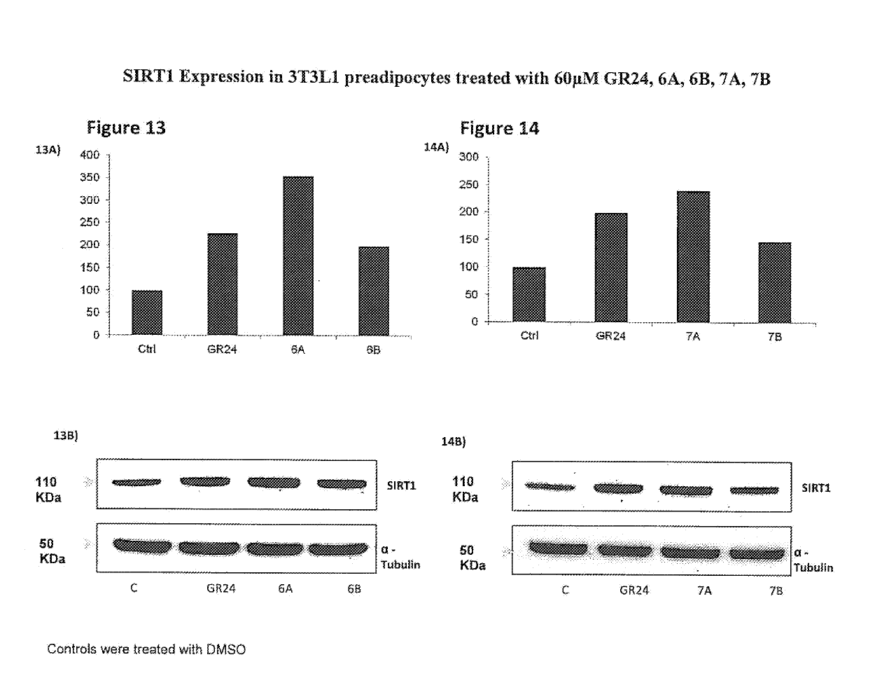

FIG. 13: SIRT1 Immunoblot and densitometry from 3T3 L1 preadipocytes treated with 60 μM GR24 or 60 μM 6A or 60 μM 6B for 24 hours. Bars show means of SIRT1 protein expression from one experiment with 2 replicates (FIG. 13A); no statistical analysis was performed. FIG. 13B shows the immunoblots of SIRT1 and α-tubulin. Quantitation of immunoblots was done by Quantity One software, and SIRT1 protein concentration was normalized to α-tubulin (used as a loading control).

FIG. 14: SIRT1 Immunoblot and densitometry from 3T3 L1 preadipocytes treated with 60 μM GR24 or 60 μM 7A or 60 μM 7B for 24 hours. Bars show means of SIRT1 protein expression from one experiment with 2 replicates (FIG. 14A); no statistical analysis was performed. FIG. 14B shows the immunoblots of SIRT1 and α-tubulin. Quantitation of immunoblots was done by Quantity One software, and SIRT1 protein concentration was normalized α-tubulin (used as a loading control).

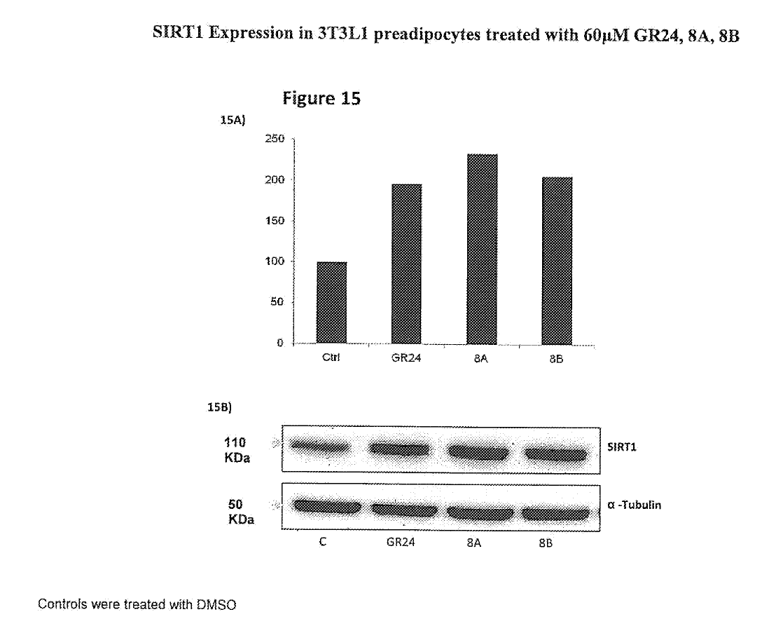

FIG. 15: SIRT1 Immunoblot and densitometry from 3T3 L1 preadipocytes treated with 60 μM GR24 or 60 μM 8A or 60 μM 8B for 24 hours. Bars show means of SIRT1 protein expression from one experiment with 2 replicates (FIG. 15A); no statistical analysis was performed. FIG. 15B shows the immunoblots of SIRT1 and α-tubulin. Quantitation of immunoblots was done by Quantity One software, and SIRT1 protein concentration was normalized to α-tubulin (used as a loading control).

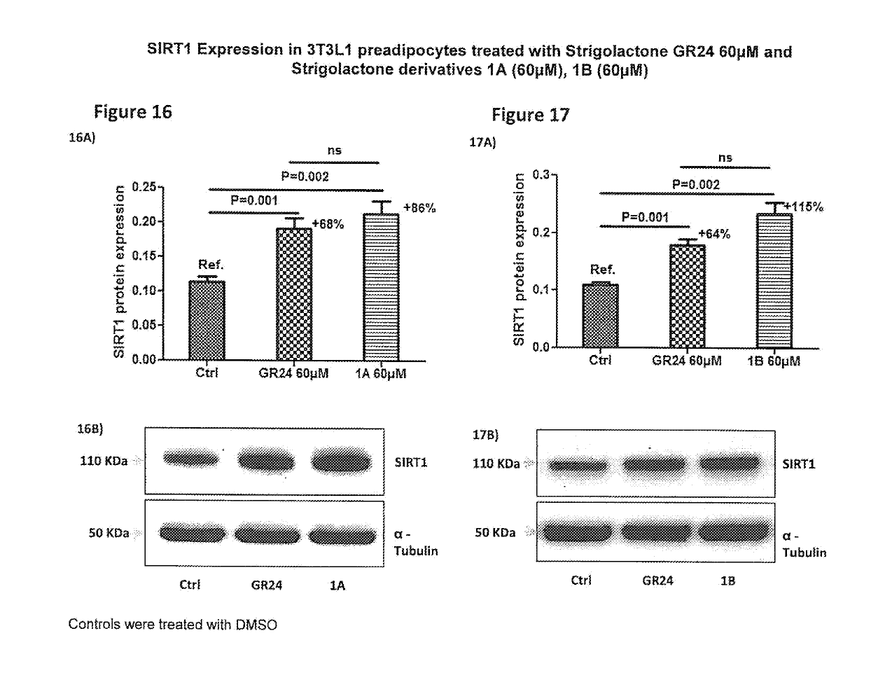

FIG. 16: SIRT1 Immunoblot and densitometry from 3T3 L1 preadipocytes treated with 60 μM GR24 or 60 μM 1A for 24 hours. Bars show mean±SEM of SIRT1 protein expression from three independent experiments, total 8 replicates (FIG. 16A). Statistical significance was assessed by pairwise t-test with correction for multiple testing (each P-value multiplied by 3). FIG. 16B shows the immunoblots of SIRT1 and α-tubulin. Quantitation of immunoblots was done by Quantity One software, and SIRT1 protein concentration was normalized to α-tubulin (used as a loading control).

FIG. 17: SIRT1 Immunoblot and densitometry from 3T3 L1 preadipocytes treated with 60 μM GR24 or 60 μM 1B for 24 hours. Bars show mean±SEM of SIRT1 protein expression from three independent experiments, total 8 replicates (FIG. 17A). Statistical significance was assessed by pairwise t-test with correction for multiple testing (each P-value multiplied by 3). FIG. 17B shows the immunoblots of SIRT1 and α-tubulin. Quantitation of immunoblots was done by Quantity One software, and SIRT1 protein concentration was normalized to α-tubulin (used as a loading control).

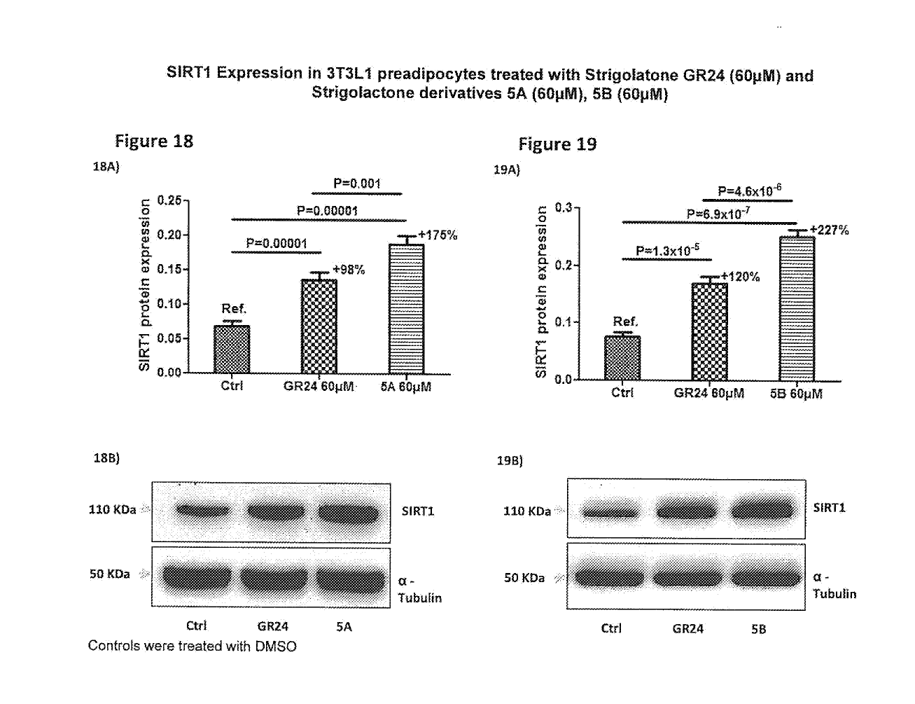

FIG. 18: SIRT1 Immunoblot and densitometry from 3T3 L1 preadipocytes treated with 60 μM GR24 or 60 μM 5A for 24 hours. Bars show mean±SEM of SIRT1 protein expression from three independent experiments, total 8 replicates (FIG. 18A). Statistical significance was assessed by pairwise t-test with correction for multiple testing (each P-value multiplied by 3). FIG. 18B shows the immunoblots of SIRT1 and α-tubulin. Quantitation of immunoblots was done by Quantity One software, and SIRT1 protein concentration was normalized to α-tubulin (used as a loading control).

FIG. 19: SIRT1 Immunoblot and densitometry from 3T3 L1 preadipocytes treated with 60 μM GR24 or 60 μM 5B for 24 hours. Bars show mean±SEM of SIRT1 protein expression from three independent experiments, total 8 replicates (FIG. 19A). Statistical significance was assessed by pairwise t-test with correction for multiple testing (each P-value multiplied by 3). FIG. 19B shows the immunoblots of SIRT1 and α-tubulin. Quantitation of immunoblots was done by Quantity One software, and SIRT1 protein concentration was normalized to α-tubulin (used as a loading control).

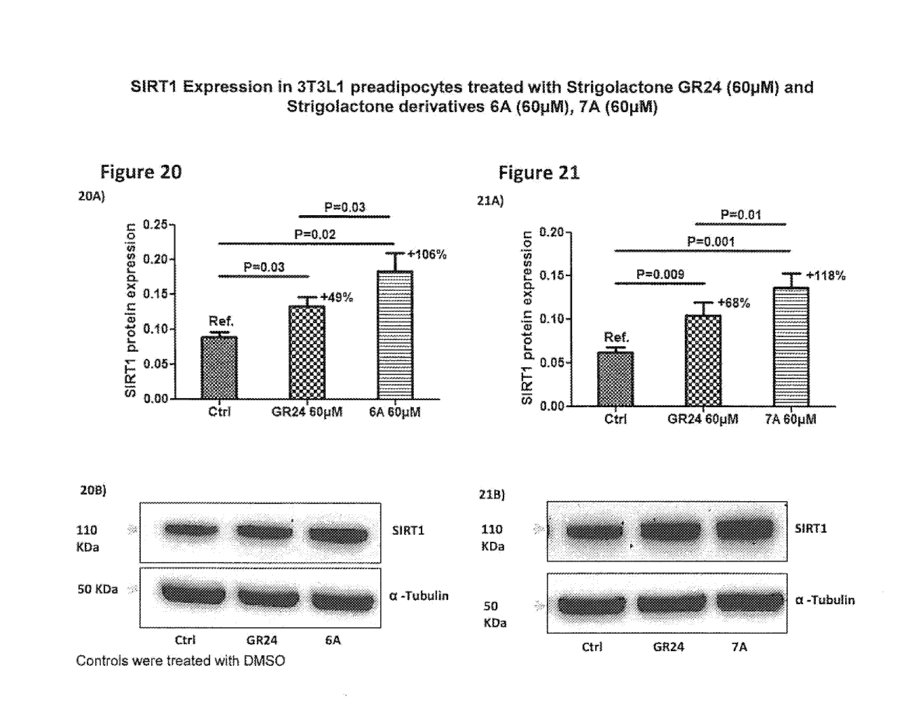

FIG. 20: SIRT1 Immunoblot and densitometry from 3T3 L1 preadipocytes treated with 60 μM GR24 or 60 μM 6A for 24 hours. Bars show mean±SEM of SIRT1 protein expression from three independent experiments, total 8 replicates (FIG. 20A). Statistical significance was assessed by pairwise t-test with correction for multiple testing (each P-value multiplied by 3). FIG. 20B shows the immunoblots of SIRT1 and α-tubulin. Quantitation of immunoblots was done by Quantity One software, and SIRT1 protein concentration was normalized to α-tubulin (used as a loading control).

FIG. 21: SIRT1 Immunoblot and densitometry from 3T3 L1 preadipocytes treated with 60 μM GR24 or 60 μM 7A for 24 hours. Bars show mean±SEM of SIRT1 protein expression from three independent experiments, total 8 replicates (FIG. 21A). Statistical significance was assessed by pairwise t-test with correction for multiple testing (each P-value multiplied by 3). FIG. 21B shows the immunoblots of SIRT1 and α-tubulin. Quantitation of immunoblots was done by Quantity One software, and SIRT1 protein concentration was normalized to α-tubulin (used as a loading control).

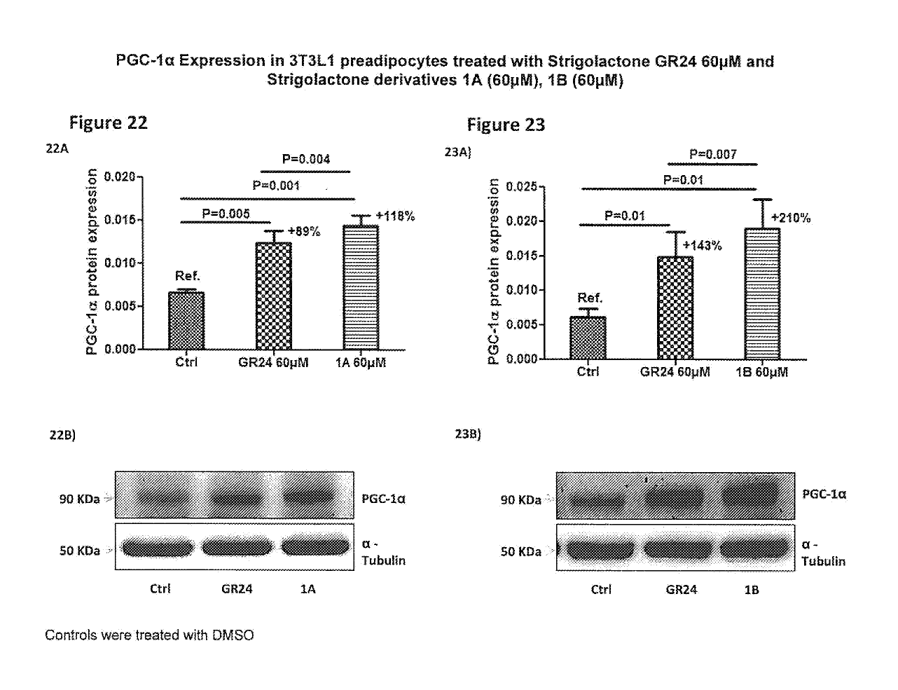

FIG. 22: PGC-1α Immunoblot and densitometry from 3T3 L1 preadipocytes treated with 60 μM GR24 or 60 μM 1A for 24 hours. Bars show mean±SEM of PGC-1a protein expression from two independent experiments, total 6 replicates (FIG. 22A). Statistical significance was assessed by pairwise t-test with correction for multiple testing (each P-value multiplied by 3). FIG. 22B shows the immunoblots of PGC-1α and α-tubulin. Quantitation of immunoblots was done by Quantity One software, and PGC-1α protein concentration was normalized to α-tubulin (used as a loading control).

FIG. 23: PGC-1α Immunoblot and densitometry from 3T3 L1 preadipocytes treated with 60 μM GR24 or 60 μM 1B for 24 hours. Bars show mean±SEM of SIRT1 protein expression from two independent experiments, total 6 replicates (FIG. 23A). Statistical significance was assessed by pairwise t-test with correction for multiple testing (each P-value multiplied by 3). FIG. 23B shows the immunoblots of PGC-1α and α-tubulin. Quantitation of immunoblots was done by Quantity One software, and PGC-1α protein concentration was normalized to α-tubulin (used as a loading control).

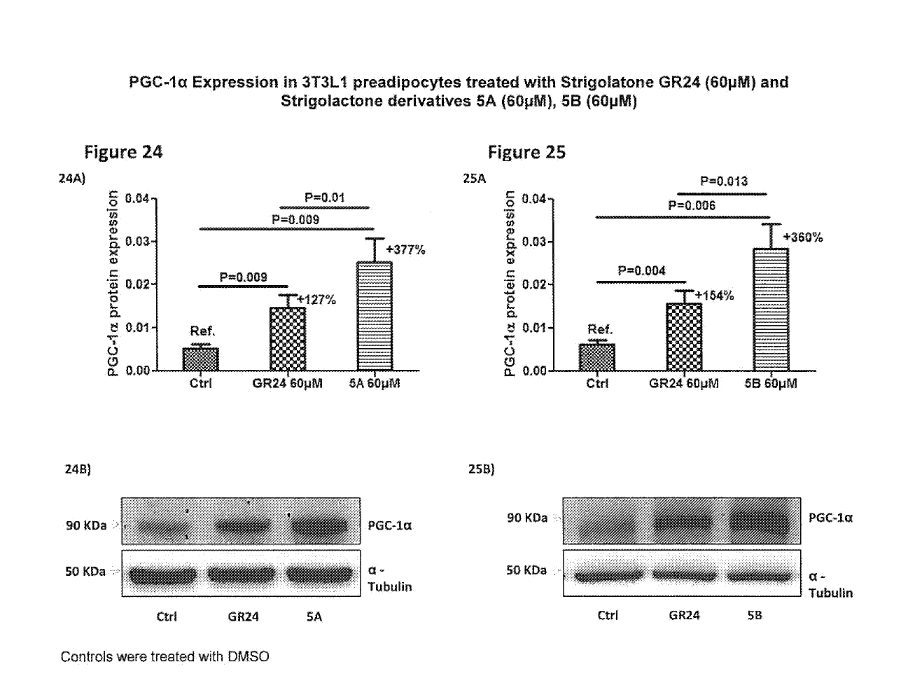

FIG. 24: PGC-1α Immunoblot and densitometry from 3T3 L1 preadipocytes treated with 60 μM GR24 or 60 μM 5A for 24 hours. Bars show mean±SEM of SIRT1 protein expression from two independent experiments, total 6 replicates (FIG. 24A). Statistical significance was assessed by pairwise t-test with correction for multiple testing (each P-value multiplied by 3). FIG. 24B shows the immunoblots of PGC-1α and α-tubulin. Quantitation of immunoblots was done by Quantity One software, and PGC-1a protein concentration was normalized to α-tubulin (used as a loading control).

FIG. 25: PGC-1α Immunoblot and densitometry from 3T3 L1 preadipocytes treated with 60 μM GR24 or 60 μM 5B for 24 hours. Bars show mean±SEM of SIRT1 protein expression from two independent experiments, total 6 replicates (FIG. 25A). Statistical significance was assessed by pairwise t-test with correction for multiple testing (each P-value multiplied by 3). FIG. 25B shows the immunoblots of PGC-1α and α-tubulin. Quantitation of immunoblots was done by Quantity One software, and PGC-1α protein concentration was normalized to α-tubulin (used as a loading control).

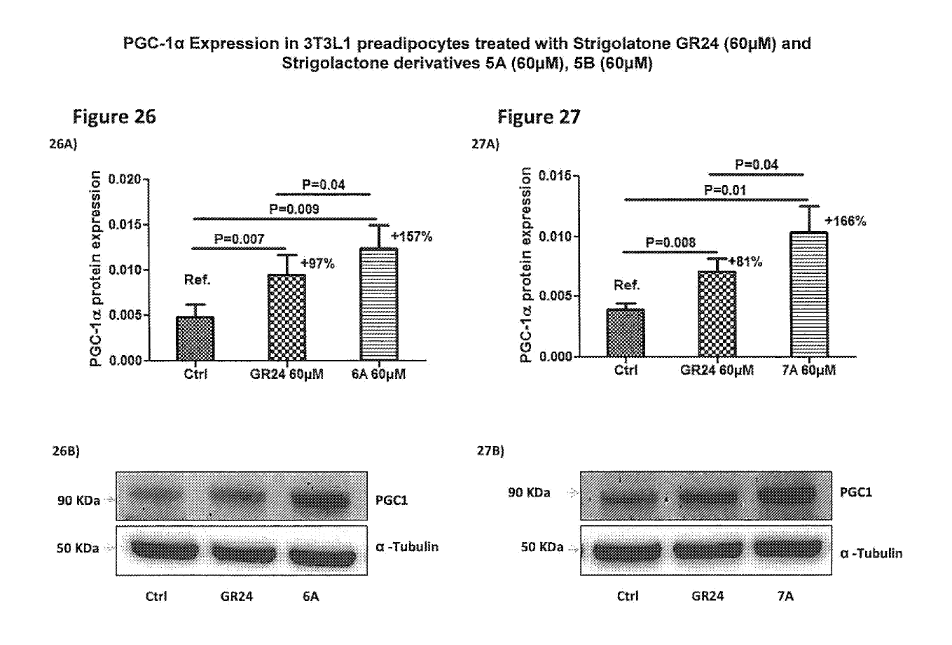

FIG. 26: PGC-1α Immunoblot and densitometry from 3T3 L1 preadipocytes treated with 60 μM GR24 or 60 μM 6A for 24 hours. Bars show mean±SEM of SIRT1 protein expression from two independent experiments, total 6 replicates (FIG. 26A). Statistical significance was assessed by pairwise t-test with correction for multiple testing (each P-value multiplied by 3). FIG. 26B shows the immunoblots of PGC-1α and α-tubulin. Quantitation of immunoblots was done by Quantity One software, and PGC-1α protein concentration was normalized to α-tubulin (used as a loading control).

FIG. 27: PGC-1α Immunoblot and densitometry from 3T3 L1 preadipocytes treated with 60 μM GR24 or 60 μM 7A for 24 hours. Bars show mean±SEM of SIRT1 protein expression from two independent experiments, total 6 replicates (FIG. 27A). Statistical significance was assessed by pairwise t-test with correction for multiple testing (each P-value multiplied by 3). FIG. 27B shows the immunoblots of PGC-1α and α-tubulin. Quantitation of immunoblots was done by Quantity One software, and PGC-1a protein concentration was normalized to α-tubulin (used as a loading control).

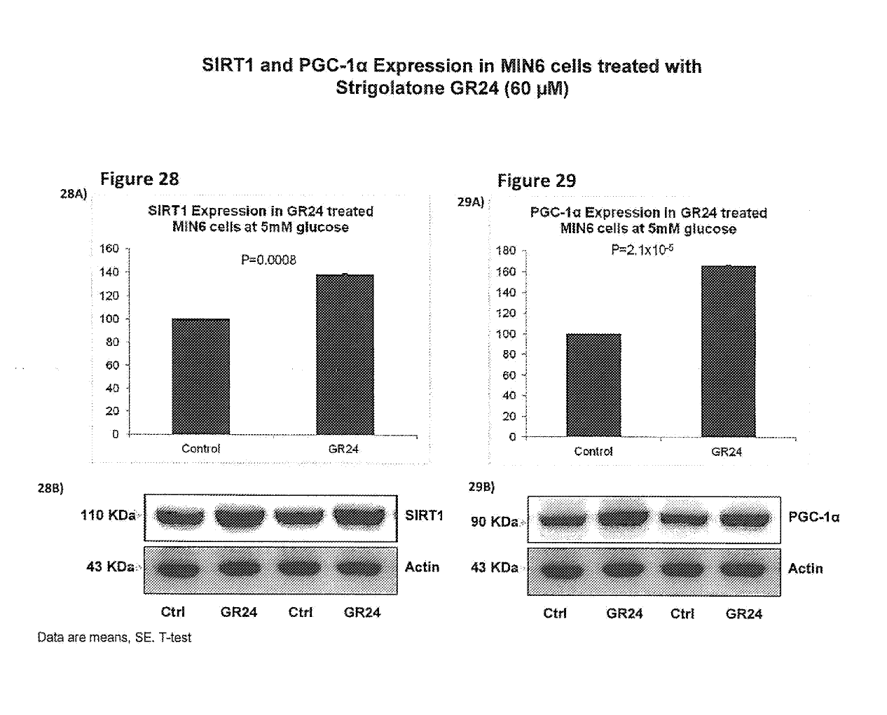

FIG. 28: SIRT1 Immunoblot and densitometry from MIN6 cells treated with 60 μM GR24 for 24 hours at 5 mM glucose. Bars show mean±SEM of SIRT1 protein expression from two independent experiments, total 6 replicates (FIG. 28A). Statistical significance was assessed by t-test. FIG. 28B shows the immunoblots of SIRT1 and Actin. Quantitation of immunoblots was done by Quantity One software, and SIRT1 protein concentration was normalized to Actin (used as a loading control).

FIG. 29: PGC-1α Immunoblot and densitometry from MIN6 cells treated with 60 μM GR24 for 24 hours at 5 mM glucose. Bars show mean±SEM of PGC-1α protein expression from two independent experiments, total 6 replicates (FIG. 29A). Statistical significance was assessed by t-test. FIG. 29B shows the immunoblots of PGC-1α and Actin. Quantitation of immunoblots was done by Quantity One software, and PGC-1a protein concentration was normalized to Actin (used as a loading control).

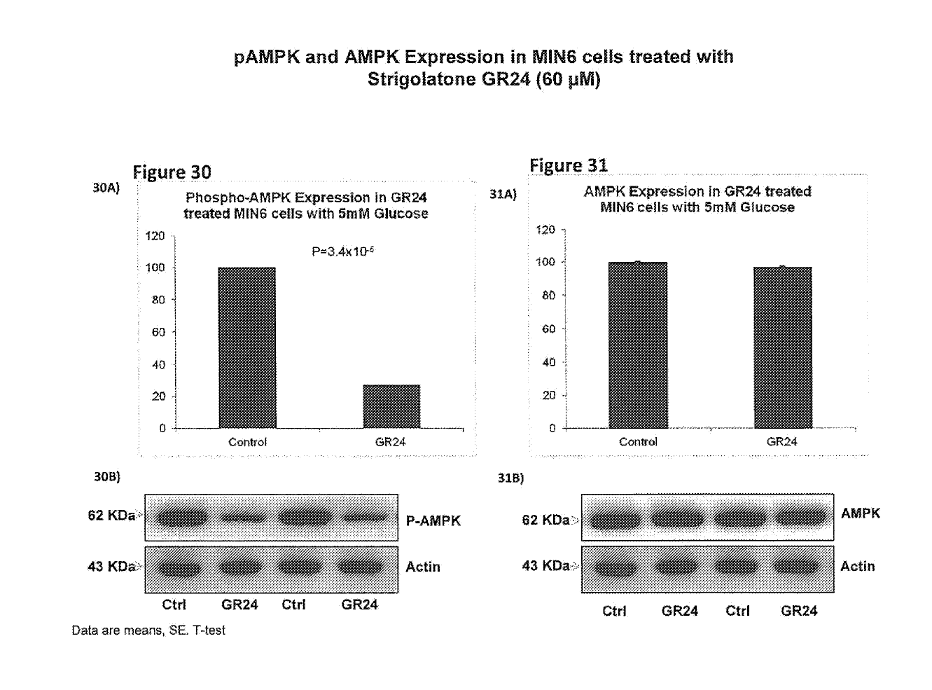

FIG. 30: pAMPK Immunoblot and densitometry from MIN6 cells treated with 60 μM GR24 for 24 hours at 5 mM glucose. Bars show mean±SEM of pAMPK protein expression from two independent experiments, total 6 replicates (FIG. 30A). Statistical significance was assessed by t-test. FIG. 30B shows the immunoblots of pAMPK and Actin. Quantitation of immunoblots was done by Quantity One software, and pAMPK protein concentration was normalized to Actin (used as a loading control).

FIG. 31: AMPK Immunoblot and densitometry from MIN6 cells treated with 60 μM GR24 for 24 hours at 5 mM glucose. Bars show mean±SEM of AMPK protein expression from two independent experiments, total 6 replicates (FIG. 31A). Statistical significance was assessed by t-test. FIG. 31B shows the immunoblots of AMPK and Actin. Quantitation of immunoblots was done by Quantity One software, and AMPK protein concentration was normalized to Actin (used as a loading control).

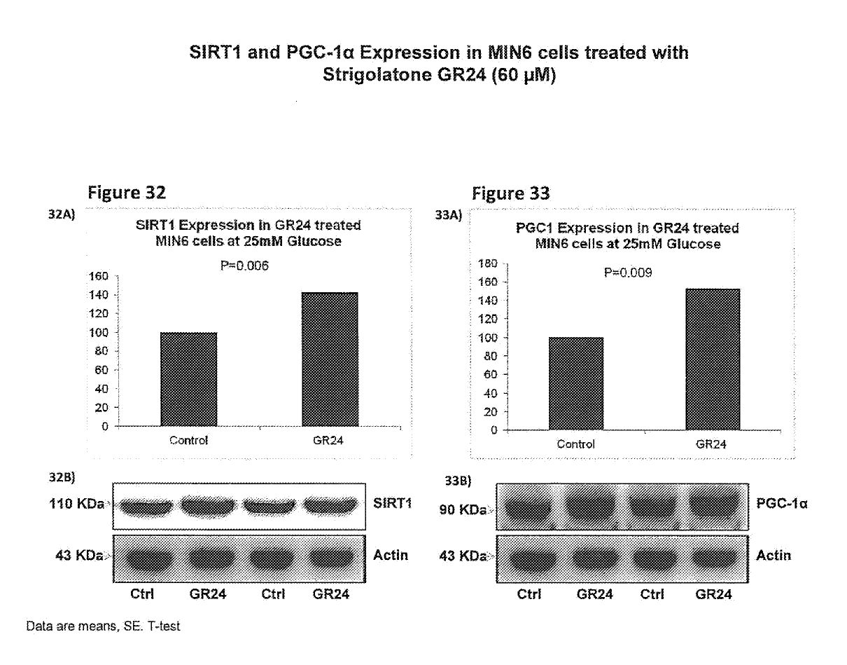

FIG. 32: SIRT1 Immunoblot and densitometry from MIN6 cells treated with 60 μM GR24 for 24 hours at 25 mM glucose. Bars show mean±SEM of SIRT1 protein expression from two independent experiments, total 6 replicates (FIG. 32A). Statistical significance was assessed by t-test. FIG. 32B shows the immunoblots of SIRT1 and Actin. Quantitation of immunoblots was done by Quantity One software, and SIRT1 protein concentration was normalized to Actin (used as a loading control).

FIG. 33: PGC-1α Immunoblot and densitometry from MIN6 cells treated with 60 μM GR24 for 24 hours at 25 mM glucose. Bars show mean±SEM of PGC-1α protein expression from two independent experiments, total 6 replicates (FIG. 33A). Statistical significance was assessed by t-test. FIG. 33B shows the immunoblots of PGC-1α and Actin. Quantitation of immunoblots was done by Quantity One software, and PGC-1a protein concentration was normalized to Actin (used as a loading control).

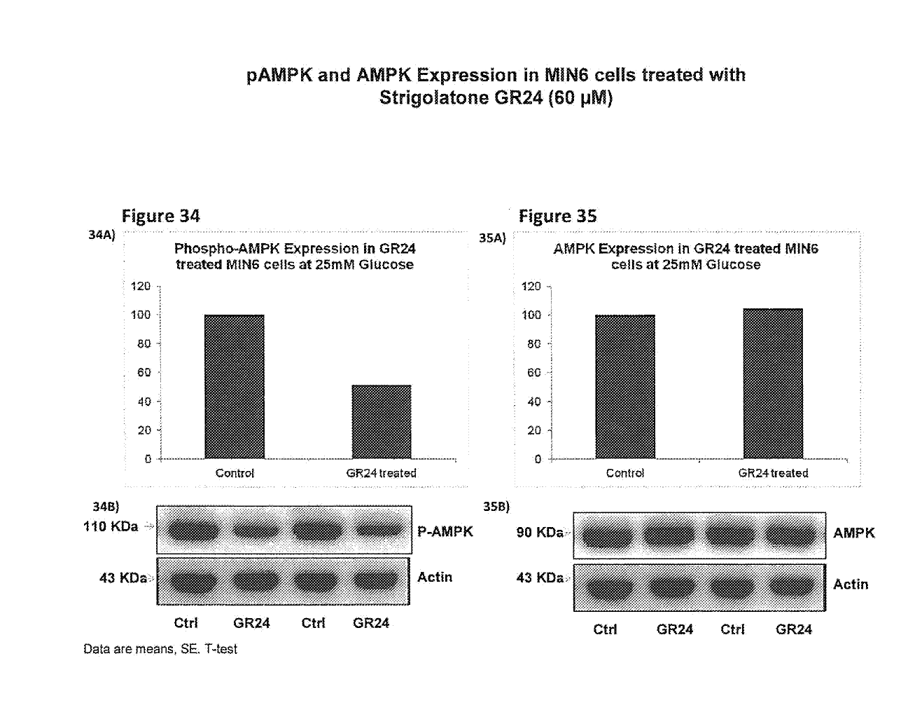

FIG. 34: pAMPK Immunoblot and densitometry from MIN6 cells treated with 60 μM GR24 for 24 hours at 25 mM glucose. Bars show mean±SEM of pAMPK protein expression from two independent experiments, total 6 replicates (FIG. 34A). Statistical significance was assessed by t-test. FIG. 34B shows the immunoblots of pAMPK and Actin. Quantitation of immunoblots was done by Quantity One software, and pAMPK protein concentration was normalized to Actin (used as a loading control).

FIG. 35: AMPK Immunoblot and densitometry from MIN6 cells treated with 60 μM GR24 for 24 hours at 25 mM glucose. Bars show mean±SEM of AMPK protein expression from two independent experiments, total 6 replicates (FIG. 35A). Statistical significance was assessed by t-test. FIG. 35B shows the immunoblots of AMPK and Actin. Quantitation of immunoblots was done by Quantity One software, and AMPK protein concentration was normalized to Actin (used as a loading control).

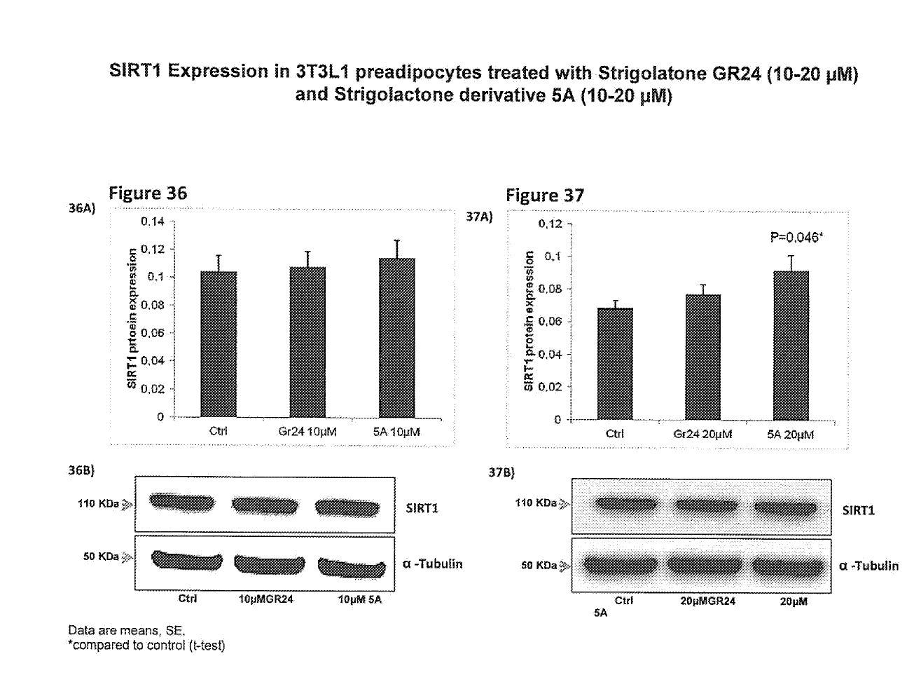

FIG. 36: SIRT1 Immunoblot and densitometry from 3T3 L1 preadipocytes treated with 10 μM GR24 or 10 μM 5A for 24 hours. Bars show mean±SEM of SIRT1 protein expression from two independent experiments, total 6 replicates (FIG. 36A). Statistical significance was assessed by t-test with correction for multiple testing (each P-value multiplied by 2). FIG. 36B shows the immunoblots of SIRT1 and α-tubulin. Quantitation of immunoblots was done by Quantity One software, and SIRT1 protein concentration was normalized to α-tubulin (used as a loading control).

FIG. 37: SIRT1 Immunoblot and densitometry from 3T3 L1 preadipocytes treated with 20 μM GR24 or 20 μM 5A for 24 hours. Bars show mean±SEM of SIRT1 protein expression from two independent experiments, total 6 replicates (FIG. 37A). Statistical significance was assessed by t-test with correction for multiple testing (each P-value multiplied by 2). FIG. 37B shows the immunoblots of SIRT1 and α-tubulin. Quantitation of immunoblots was done by Quantity One software, and SIRT1 protein concentration was normalized to α-tubulin (used as a loading control).

Results are summarized in Tables 1 and 2, below.

Table 1 indicates the percentage changes in SIRT1 protein expression in 3T3 L1 preadipocytes treated with 60 μM GR24 or 60 μM 1A, 1B, 5A, 5B, 6A, 7A for 24 hours compared to control (100%) (Columns “GR24” and “Derivative”), or derivative compared to GR24 (=100%) (the last column).

Table 2 indicates the percentage changes in PGC-1α protein expression in 3T3 L1 preadipocytes treated with 60 μM GR24 or 60 μM 1A, 1B, 5A, 5B, 6A, 7A for 24 hours compared to control (100%) (columns “GR24” and “Derivative”), or derivative compared to GR24 (=100%) (the last column).

TABLE 1
Percentage Changes in SIRT1 Expression with Treatments:

| Control | GR24 | Derivative | Derivative vs. GR24 |
|---|---|---|---|
| 100% | 168% | 1A 186% | +11% |
| 100% | 164% | 1B 215% | +31% |
| 100% | 198% | 5A 275% | +39% |
| 100% | 220% | 5B 327% | +48% |
| 100% | 149% | 6A 206% | +38% |
| 100% | 168% | 7A 218% | +30% |

TABLE 2
Percentage Changes in PGC-1α with Treatments:

| Control | GR24 | Derivative | Derivative vs. GR24 |
|---|---|---|---|
| 100% | 168% | 1A 186% | +11% |
| 100% | 164% | 1B 215% | +31% |
| 100% | 198% | 5A 275% | +39% |
| 100% | 220% | 5B 327% | +48% |
| 100% | 149% | 6A 206% | +38% |
| 100% | 168% | 7A 218% | +30% |

1.-9. (canceled)

10. A composition comprising a combination of a terpenoid lactone that is a selective activator of SIRT1 and an additional SIRT1 activator selected from stilbenoids, flavonoids, chalconoids, tannins, and nicotinamide inhibition antagonists.

11. The composition of claim 10, wherein the additional SIRT1 activator is a stilbenoid.

12. The composition of claim 11, wherein the stilbenoid has the structure of formula (II) 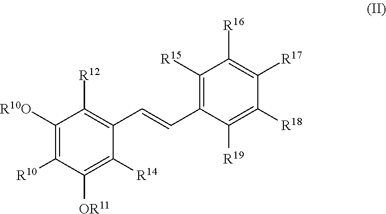
wherein:
R¹⁰ is selected from hydrogen, C₁-C₆ alkyl, halogenated C₁-C₆ alkyl, C₂-C₆ acyl, and a glycoside;
R¹¹ is selected from hydrogen, C₁-C₆ alkyl, halogenated C₁-C₆ alkyl, and C₂-C₆ acyl;
R¹², R¹⁴, R¹⁵, and R¹⁹ are independently selected from hydrogen, halo, C₁-C₆ alkyl, and halogenated C₁-C₆ alkyl; and
R¹³, R¹⁶, R¹⁷, and R¹⁸ are independently selected from hydrogen and OR²⁰, where R²⁰ is hydrogen, C₁-C₆ alkyl, halogenated C₁-C₆ alkyl, or C₂-C₆ acyl;
or is an oligomer or glycoside thereof.

13. The composition of claim 12, wherein R¹², R¹⁴, R¹⁵, and R¹⁹ are hydrogen.

14. The composition of claim 13, wherein R¹⁰ and R¹¹ are independently selected from hydrogen and C₁-C₆ alkyl.

15. The composition of claim 14, wherein R¹⁰ and R¹¹ are both hydrogen.

16. The composition of claim 14, wherein R¹⁰ and R¹¹ are both methyl.

17. The composition of claim 14, wherein R²⁰ is hydrogen or C₁-C₆ alkyl.

18. The composition of claim 11, wherein the stilbenoid is an oligomer.

19. The composition of claim 18, wherein the oligomer is a trimer or tetramer.

20. The composition of claim 10, further comprising a pharmaceutically acceptable carrier.

21. The composition of claim 20, wherein the composition is an orally administrable dosage form.

22. The composition of claim 21, wherein the dosage form provides for controlled release of at least the terpenoid.

23. The composition of claim 21, wherein the dosage form is a tablet.

24. The composition of claim 21, wherein the dosage form is a capsule.

25. A composition comprising GR 24 and resveratrol.

26-27. (canceled)

28. A method for influencing energy metabolism in a eukaryotic cell, comprising contacting the eukaryotic cell with a terpenoid lactone that is a selective activator of SIRT1 in an amount effective to influence energy metabolism.

29. The method of claim 28, wherein the eukaryotic cell is a mammalian cell.

30. The method of claim 29, wherein the terpenoid lactone comprises a dilactone.

31. The method of claim 30, wherein the terpenoid lactone contains a 5-alkenyloxy-furan-2-one group.

32. The method of claim 31, wherein the terpenoid lactone has the structure of formula (I) 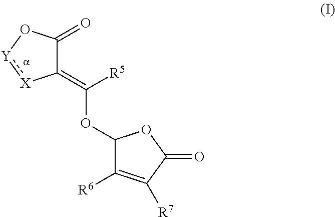
wherein:
α is an optionally present double bond;
when α is present, such that X and Y are linked through a double bond, X is CR¹ and Y is CR³;
when α is absent, such that X and Y are linked through a single bond, X is selected from CR¹R² and CR¹R²—CR⁸R⁹, and Y is CR³R⁴;
R¹, R², R³, R⁴, R⁸, and R⁹ are independently selected from hydrogen, halo, hydroxyl, sulfhydryl, C₁-C₂₄ alkoxy, C₂-C₂₄ alkenyloxy, C₂-C₂₄ alkynyloxy, C₅-C₂₄ aryloxy, C₂-C₂₄ alkylcarbonyl, C₆-C₂₄ arylcarbonyl, C₂-C₂₄ alkylcarbonyloxy, C₆-C₂₄ arylcarbonyloxy, halocarbonyl, C₂-C₂₄ alkylcarbonato, C₆-C₂₄ arylcarbonato, carboxy, carboxylato, carbamoyl, mono-(C₁-C₂₄ alkyl)-substituted carbamoyl, di-(C₁-C₂₄ alkyl)-substituted carbamoyl, mono-(C₆-C₂₄ aryl)-substituted carbamoyl, thiocarbamoyl, carbamido, cyano, isocyano, cyanato, isocyanato, isothiocyanato, azido, formyl, thioformyl, amino, mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido, C₆-C₂₄ arylamido, imino, alkylimino, arylimino, nitro, nitroso, sulfo, sulfonato, C₁-C₂₄ alkylthio, C₅-C₂₄ arylthio, C₁-C₂₄ alkylsulfinyl, C₅-C₂₄ arylsulfinyl, C₁-C₂₄ alkylsulfonyl, C₅-C₂₄ arylsulfonyl, phosphono, phosphonato, phosphinato, phosphono, phosphino, C₁-C₂₄ alkyl, C₂-C₂₄ alkenyl, C₂-C₂₄ alkynyl, C₅-C₂₄ aryl, C₆-C₂₄ alkaryl, and C₆-C₂₄ aralkyl, and further wherein R¹ and R³, and R¹ and R⁸ may be taken together to form a cyclic structure selected from a five-membered ring and a six-membered ring, optionally fused to an additional five-membered or six-membered ring, wherein the rings are aromatic, alicyclic, heteroaromatic, or heteroalicyclic, and have zero to 4 non-hydrogen substituents and zero to 3 heteroatoms;
R⁵ is selected from hydrogen, halo, C₁-C₆ alkyl, substituted C₁-C₆ alkyl, C₁-C₆ heteroalkyl, and substituted C₁-C₆ heteroalkyl; and
R⁶ and R⁷ are independently selected from hydrogen, halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, or R⁶ and R⁷ may be taken together to form a C₅-C₁₄ cyclic group, optionally substituted and/or containing at least one heteroatom.

33. The method of claim 28, further comprising contacting the cell with an additional SIRT1 activator selected from stilbenoids, flavonoids, chalconoids, tannins, and nicotinamide inhibition antagonists.

34. The method of claim 33, wherein the cell is simultaneously contacted with the terpenoid lactone and the additional SIRT1 activator.

35. A compound having the structure of formula (I) 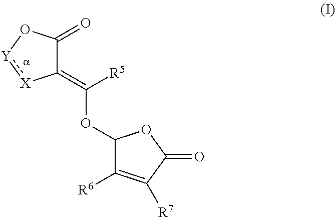
wherein:
α is an optionally present double bond;
when α is present, such that X and Y are linked through a double bond, X is CR¹ and Y is CR³;
when α is absent, such that X and Y are linked through a single bond, X is selected from CR¹R² and CR¹R²—CR⁸R⁹, and Y is CR³R⁴;
R¹, R², R³, R⁴, R⁸, and R⁹ are independently selected from hydrogen, halo, hydroxyl, sulfhydryl, C₁-C₂₄ alkoxy, C₂-C₂₄ alkenyloxy, C₂-C₂₄ alkynyloxy, C₅-C₂₄ aryloxy, C₂-C₂₄ alkylcarbonyl, C₆-C₂₄ arylcarbonyl, C₂-C₂₄ alkylcarbonyloxy, C₆-C₂₄ arylcarbonyloxy, halocarbonyl, C₂-C₂₄ alkylcarbonato, C₆-C₂₄ arylcarbonato, carboxy, carboxylato, carbamoyl, mono-(C₁-C₂₄ alkyl)-substituted carbamoyl, di-(C₁-C₂₄ alkyl)-substituted carbamoyl, mono-(C₆-C₂₄ aryl)-substituted carbamoyl, thiocarbamoyl, carbamido, cyano, isocyano, cyanato, isocyanato, isothiocyanato, azido, formyl, thioformyl, amino, mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido, C₆-C₂₄ arylamido, imino, alkylimino, arylimino, nitro, nitroso, sulfo, sulfonato, C₁-C₂₄ alkylthio, C₅-C₂₄ arylthio, C₁-C₂₄ alkylsulfinyl, C₅-C₂₄ arylsulfinyl, C₁-C₂₄ alkylsulfonyl, C₅-C₂₄ arylsulfonyl, phosphono, phosphonato, phosphinato, phosphono, phosphino, C₁-C₂₄ alkyl, C₂-C₂₄ alkenyl, C₂-C₂₄ alkynyl, C₅-C₂₄ aryl, C₆-C₂₄ alkaryl, and C₆-C₂₄ aralkyl, and further wherein R¹ and R³, and R¹ and R⁸ may be taken together to form a cyclic structure selected from a five-membered ring and a six-membered ring, optionally fused to an additional five-membered or six-membered ring, wherein the rings are aromatic, alicyclic, heteroaromatic, or heteroalicyclic, and have zero to 4 non-hydrogen substituents and zero to 3 heteroatoms;
R⁵ is selected from hydrogen, halo, C₁-C₆ alkyl, substituted C₁-C₆ alkyl, C₁-C₆ heteroalkyl, and substituted C₁-C₆ heteroalkyl; and
(a) R⁶ and R⁷ taken together form a C₅-C₁₄ cyclic group, optionally substituted and/or containing at least one heteroatom; or
(b) R⁶ is hydrogen and R⁷ is selected from halo, hydroxy, C₁-C₁₂ alkoxy, C₂-C₁₂ hydrocarbyl, substituted C₂-C₁₂ hydrocarbyl, heteroatom-containing C₂-C₁₂ hydrocarbyl, and substituted heteroatom-containing C₂-C₁₂ hydrocarbyl; or
(c) R⁶ is selected from halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, and R⁷ is selected from hydrogen, halo, hydroxy, C₁-C₁₂ alkoxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, wherein R⁶ and R⁷ may be the same or different.

36. The compound of claim 34, wherein R⁶ and R⁷ are taken to form a C₅-C₁₄ cyclic group, optionally substituted and/or containing at least one heteroatom.

37. The compound of claim 36, wherein the cyclic group is monocyclic or bicyclic.

38. The compound of claim 36, wherein the cyclic group is aromatic.

39. The compound of claim 38, wherein the cyclic group is a phenyl ring.

40. The compound of claim 35, wherein R⁶ is hydrogen and R⁷ is C₂-C₁₂ hydrocarbyl, optionally substituted and/or heteroatom-containing.

41. The compound of claim 40, wherein R⁷ is C₂-C₆ alkyl.

42. The compound of claim 35, wherein R⁶ and R⁷ are optionally substituted, optionally heteroatom-containing C₁-C₁₂ alkyl, and may be the same or different.

43. The compound of claim 42, wherein R⁶ and R⁷ are optionally substituted, optionally heteroatom-containing C₁-C₆ alkyl.

44. A compound comprising the structure of formula (VIII) 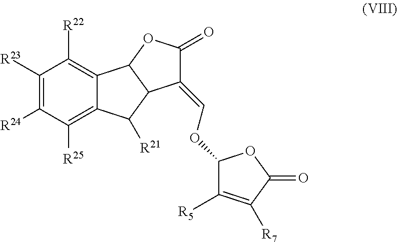
wherein:
R⁶ and R⁷ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, C₁-C₁₂ hydrocarbyl, substituted C₁-C₁₂ hydrocarbyl, heteroatom-containing C₁-C₁₂ hydrocarbyl, and substituted heteroatom-containing C₁-C₁₂ hydrocarbyl, or R⁶ and R⁷ may be taken together to form a C₅-C₁₄ cyclic group, optionally substituted and/or containing at least one heteroatom;
R²¹ is selected from hydrogen, hydroxy, C₁-C₃ alkoxy, and C₂-C₄ acyloxy; and either
(a) one of R²², R²³, R²⁴, and R²⁵ is C₁-C₁₂ hydrocarbyl, optionally substituted and optionally heteroatom-containing, and the others are hydrogen; or
(b) R²², R²³, R²⁴, and R²⁵ are independently selected from hydrogen, halo, hydroxy, C₁-C₁₂ hydrocarbyloxy, substituted C₁-C₁₂ hydrocarbyloxy, heteroatom-containing C₁-C₁₂ hydrocarbyloxy, substituted heteroatom-containing C₁-C₁₂ hydrocarbyloxy, substituted C₁-C₁₂ hydrocarbyl, heteroatom-containing C₁-C₁₂ hydrocarbyl, and substituted heteroatom-containing C₁-C₁₂ hydrocarbyl, with the proviso that at least one of R²², R²³, R²⁴, and R²⁵ is optionally substituted, optionally heteroatom-containing C₁-C₁₂ hydrocarbyloxy.

45-46. (canceled)

47. A method of treating or preventing a metabolic disorder comprising administering to a subject in need thereof a terpenoid lactone that is a selective activator of SIRT1.

48. (canceled)

49. A method of treating or preventing a disorder associated with energy metabolism, mitochondrial activity and/or the aging process of an organism comprising administering to the organism in need thereof a terpenoid lactone that is a selective activator of SIRT1.

50. (canceled)
